(19) United States
(12) Patent Application Publication
(10) Pub. No.: US 2004/0019921 A1
(43) Pub. Date: Jan. 29, 2004

(54) Non-human mammal with disrupted or modified MIF gene, and uses thereof

(76) Inventors: Gunter R. Fingerle-Rowson, Long Beach, NY (US); Patrick R. Delaney, (US)

Correspondence Address: PIPER RUDNICK LLP, Supervisor, Patent Prosecution Services, 1200 Nineteenth Street, N.W., Washington, DC, 20036-2412, US

(21) Appl. No.: 10/322,685

(22) Filed: Dec. 19, 2002

Related U.S. Application Data
(60) Provisional application No. 60/340,956, filed on Dec. 19, 2001.

(51) Int. Cl. A01K 67/027

(52) U.S. Cl. 800/18

(57) ABSTRACT

The present invention demonstrates transgenic mammals, particularly transgenic mice, having a genomic disruption or mutation affecting the MIF gene. The invention is also directed to use of the transgenic mice in developing therapies to inflammatory or neoplastic disorders involving MIF cellular activity.

This application takes priority from Provisional Application No. 60/340,956, filed Dec. 19, 2002, the entirety of which, and all references cited or listed herein, are incorporated by reference herein for all purposes.

### BACKGROUND OF THE INVENTION

1. Field of the Invention

The present invention relates generally to animal models that are useful for studying the role of macrophage migration inhibitory factor (MIF) in cellular activity governing proinflammatory responses and cell cycle disorders, and for development of therapies for inflammatory and neoplastic disorders. In particular, the invention relates to mice in which the gene encoding macrophage migration inhibitory factor (MIF) has been deleted, nullified or mutated. More particularly, the following mouse strains are described: C57B1/6J-TgH (MIFflox) 1 Grf mouse (“MIF flox-mouse”) which can be used, among other things, to generate inducible and tissue-specific MIF knockout mice; C57B1/6J-TgH(MIFdel)2Grf mouse (“MIF knockout mouse”); C57B1/6J-TgH(MIFpg)3Grf mouse (“MIF plg-mouse”), comprising a mutation of proline 1 of MIF; and C57B1/6J-TgH(MIFcs)4Grf mouse (“MIF c60s-mouse”) comprising a mutation of cysteine 60 of MIF.

To provide these animal models, the MIF gene is disrupted or mutated in mice, that is, a disrupted or null mutation in the mouse MIF gene is engineered. Mice in which the MIF gene is nullified are called “knockout” mice, whereas mice in which a specific mutation is engineered are “knock-in” models. The knockout model can be produced by the Cre-loxP technique on a pure C57B1/6 background using embryonic stem (ES) cell clones in which the MIF gene is either flanked by loxP sites (i.e, “floxed”) or deleted. Knock-in models were also developed to study the potential effect of different enzymatic activities of MIF. Mutation by a single amino-acid substitution arrests potential isomerase or oxidoreductase enzymatic activities of MIF in oncongenic transformation and normal ES cell growth. These animal models having a knock-in mutation allow further insight into the molecular action of MIF in developing therapies for both inflammatory and neoplastic disorders.

The MIF knockout animal model, and in particular, the MIF knock-out mouse according to the invention provides a platform for developing monoclonal or polyclonal antibodies to human MIF having fewer background byproducts associated with human MIF antibody production than for anti-human MIF antibodies prepared in a wild-type mouse. The MIF flox-mouse model is a unique and useful intermediate in preparing the MIF knock-out mouse model as it allows preparation of an MIF knockout model with a unique genome as compared to other MIF knock-out mice known in the art.

Also, the MIF knock-in animal models according to the invention, and in particular, MIF knock-in mice, provide in vivo systems for screening to find MIF inhibitors that do not inhibit the MIF enzymatic activity affected by the mutation in the knock-in model. The MIF knock-in models also provide a platform to test for drugs that affect MIF but do not inhibit the enzymatic activity of MIF in vivo.

The biological activities of MIF and its upregulation in inflammatory and neoplastic diseases suggest that MIF may be involved in the pathogenesis of these common and often lethal conditions. New therapeutic approaches are urgently needed to improve existing treatment options for these diseases, but they also require a thorough understanding of the biology and mechanism of action of the selected target. Anti-MIF antibodies demonstrated the potential usefulness of MIF as a therapeutic target, but the biology of MIF is still incompletely understood. The animal models according to the invention allow investigation of the biological role of MIF by genetic means.

### DISCUSSION OF THE BACKGROUND

In response to antigenic or mitogenic stimulation, lymphocytes secrete protein mediators called lymphokines that play important roles in immunoregulation, inflammation and effector mechanisms of cellular immunity, (346). The first reported lymphokine activity was observed in culture supernatants of antigenically sensitized and activated guinea pig lymphocytes. This activity was named migration inhibitory factor (MIF) for its ability to prevent the migration of guinea pig macrophages in vitro, (347). The detection of MIF activity is correlated with a variety of inflammatory responses including delayed hypersensitivity and cellular immunity, (348); allograft rejection, and rheumatoid polyarthritic synovialis, (349).

MIF is a lymphokine produced by activated T cells and is a major secreted protein released by the anterior pituitary cells. Many publications have reported isolation and identification of putative MIF molecules. For example, human MIF-1 was purified to homogeneity from serum-free culture supernatant of a human T cell hybridoma clone called F5,(350). Human MIF-2, which is more hydrophobic than human MIF-1, was purified to homogeneity from the same clone, (351). Human MIF-3 is structurally related to the MIF family and is reported in U.S. Pat. No. 5,986,060. The patent reports that the human MIF-3 variant is functionally similar in many mechanisms to human MIF-1 and is involved in mechanisms affecting neoplastic and inflammatory disorders.

MIFs of about 13 kilodaltons (kD) have been identified in several mammalian and avian species; see, for example, (352-356). Although MIF was first characterized as blocking macrophage migration, MIF also appears to affect macrophage-macrophage adherence, induce macrophage to express interleukin-1-beta, interleukin-6, and tumor necrosis factor alpha; and up-regulate HLA-DR as well as other activities.

The human MIF-1 and mouse MIF genes were cloned and sequenced in 1994 (35) and 1995 (27) respectively. A distantly related protein called D-dopachrome tautomerase, which has 29% amino acid sequence identity with MIF in the mouse, was cloned in 1998 and shown to exhibit a similar tertiary structure (125; 128; 228).

The presence of MIF homologues in a wide variety of species suggests that MIF is an evolutionary ancient molecule and is likely to participate in basic cellular functions common to all these diverse organisms. Furthermore, the presence of three homologous proteins in C. elegans, as well as the structural similarity of MIF with D-dopachrome tautomerase (DT), supports the idea that MIF has evolved as part of a protein superfamily, with some members yet unknown.

The human MIF-1 gene is located on chromosome 22q11.2 and in the mouse on chromosome 10 between the Bcr and the S100b loci (29;30). Mouse mif is located next to matrix metalloproteinase-11 (mmp 11 or stromelysin 3), two glutathione-S-transferases genes and D-dopachrome tautomerase. The structural similarity of the genomic organization and of the proteins suggests the mif-gsst cluster might have arisen as the result of a duplication event.

The technique of gene targeting for inactivating a single gene of interest has found widespread acceptance in biomedical research. This technique provides a powerful test of the function of a gene by damaging it to the extent that it cannot produce a functional protein. The resulting mice then are observed and tested for physiological abnormalities. The Cre-loxP technique of gene targeting avoids embryonic lethality, and it offers the possibility of inactivating the gene in a conditional or even cell type-specific manner (202).

MIF was recognized as far back as the late 1950s to be associated with immune activation (3;4). The technological progress and new discoveries of the late 1980s bring this molecule back into the scientific spotlight. MIF was shown to be a proinflammatory cytokine playing a major role in septic shock and counter-regulating the anti-inflammatory effects of glucocorticoids (69;245). Antibodies to MIF inhibite disease progression and improve the outcome in several animal models of inflammatory diseases such as septic shock (245), arthritis (246), and glomerulonephritis (247). These discoveries with potential relevance to human disease led to increased efforts to better understand MIF in biological mechanisms. The almost ubiquitous expression of MIF, its developmental regulation (13) as well as its association with the regulation of cellular proliferation and neoplastic disease (238), however, suggest that MIF might have functions beyond the immune system.

In 1992, MIF was described as a serum-inducible, delayed early response gene in a cell line (BALBc/3T3 fibroblasts) (80). Delayed early response genes are believed to be induced by immediate-early response genes (e.g., c-fos, c-jun, c-myc) within a few hours but still prior to the onset of DNA synthesis. Delayed-early response genes constitute a very heterogeneous group of genes, which participate in a variety of cellular processes such as biosynthetic pathways (e.g., omithine decarboxylase in synthesis of polyamines), cell to cell interaction (e.g., proliferin, PLF) (248), or transcriptional regulation (e.g. cyclin D). Additional evidence for the role of MIF in growth and proliferation is provided by the findings that MIF expression correlated with lens cell (81) and embryonic development (13), and that inhibition of MIF inhibits proliferation of activated T-lymphocytes (16), angiogenesis (82) and growth of tumor cell lines (249).

Upon mitogenic stimulation, mammalian cells progress from the resting phase (G0) into the initial phase (G1). Once a threshold size has been reached and specific proteins are activated, cells in G1 can enter into S phase. The commitment to DNA synthesis occurs at a restriction point late in the G1 phase, after which mitogenic signals are no longer required for cells to progress. After the DNA has been replicated, cells progress through another much shorter growth phase, termed G2. This phase has several regulatory mechanisms that ensure that the genome has been replicated only once. Finally, the cell is ready to go through mitosis (M-phase) and to divide into two identical cells, which will start a new cell cycle (250).

The orderly progression through these phases is ensured by timely regulated expression of cyclins. Cyclins are activators of a family of protein serine/threonine kinases, the cyclin-dependent kinases (CDKS) which activate or inactivate downstream effectors through phosphorylation (251). The best-characterized substrates of cyclin-CDK complexes are the retinoblastoma family proteins pRb, p107 and p130, which in their hypo-phosphorylated form repress the activity of several targets such as the heterodimeric E2F-DP transcription factors. Five E2F (E2F 1-5) and three DP proteins (DP 1-3) have been identified that differ in their binding affinities and tissue distribution. Depending on the target promoters, E2F complexes can function as either transcriptional activators or repressors. E2F target genes include regulators of S-phase entry (e.g., B-myb, CDC2, cyclins E and A) and genes required for DNA replication (e.g., dihydrofolatereductase, DNA polymerase a, thymidine kinase) (252).

The G1/S cell cycle checkpoint controls the passage of eukaryotic cells from the G1 phase into the DNA synthesis phase. Two cell cycle kinases, CDK4/6-cyclin D and CDK2-cyclin E and the transcription complex that includes Rb and E2F are pivotal in controlling this checkpoint. During G1-phase, the Rb-Histone deacetylase repressor complex binds to the E2F-DPI transcription factors, inhibiting downstream transcription. Phosphorylation of Rb by CDK4/6 and CDK2 dissociates the Rb-repressor complex, permitting transcription of S-phase genes. Besides association with cyclins, the activity of CDKs is controlled by site-specific phosphorylation or dephosphorylation and by association with a group of inhibitory proteins collectively called cyclin-dependent kinase inhibitors (CKIs) (89). Many different stimuli exert checkpoint control including TGF-β, DNA damage, contact inhibition, replicative senescence, and growth factor withdrawal. The first four act by inducing members of the INK4 or Kip/Cip families of CKIs. TGF-β additionally inhibits the transcription of Cdc25A, a phosphatase that activates the cell cycle kinases. Growth factor withdrawal activates GSK30, which 4 phosphorylates cyclin D, leading to its rapid ubiquitination and proteosomal degradation (253). Dilution, ubiquitination, nuclear export and degradation are mechanisms commonly used to rapidly reduce the concentration of cell-cycle control proteins.

Recent years have witnessed the generation of many mice deficient in cell cycle regulators. While many of them exhibit strong phenotypes such as embryonic lethality (e.g., Rb (254)), gigantism (e.g., p27Kip1 (255)), dwarfism (e.g., cyclin D (256;257)), lethal inflammation (e.g., TGF-β (258)) or increased incidence of tumors (e.g., p53 (259)), the phenotype of others may be somewhat hidden and challenging to find (e.g., CDC25C (260), p130 (261;262), p21Cip1 (263), p16INK4a (264)). MIF^{-/-} mice have been developed using a 129/Sv background (236, 237). These appear to belong to the latter group phenotype, as they are normal with respect to size, weight, morphology, organ development, fertility or incidence of spontaneous tumors. MIF^{-/-} mice have been reported to be more susceptible to Leishmania major infection (265).

Growth and malignant transformation are interrelated processes and often involve the same regulating pathways and molecules. The influence of oncogenes (e.g. adenoviral E1A or H-ras) or proto-oncogenes (e.g. c-myc) on growth and cell cycle control is subject of intense research due to its profound implications for the biology of cancer.

Ras is a key regulator of cell growth in all eukaryotic cells. Genetic, biochemical and molecular studies have positioned Ras centrally in signal transduction pathways that respond to diverse extracellular stimuli, including growth factors, cytokines and hormones. Ras proteins exist in two conformations, a GTP-bound active state and a GDP-bound inactive state: the ratio of GTP to GDP bound to cellular Ras proteins is controlled by guanine nucleotide exchange factors (GEFs) and GTPase-activating proteins (GAPS) (277). Mutations in Ras at amino acids 12, 13 or 61 make Ras insensitive to GAP action and, hence, constitutively active in transforming mammalian cells (241). It has been estimated that 30% of all human tumors contain an activating mutation in Ras. The frequency of Ras mutations varies depending on tumor type, with the highest frequencies seen in lung (30%), colon (50%), thyroid (50%) and pancreatic carcinomas (90%). Ras mediates its effects through multiple effectors. One central pathway is activated by direct binding to Raf, which triggers a cascade of kinases (Raf-MEK-ERK). However, activated Raf is not sufficient to promote all functions of Ras, such as the transformation of some epithelial cells (278). A plethora of candidate Ras effectors in addition to Raf has been reported which activate multiple effector pathways and contribute to Ras function. These include p120 Ras GAP (279), GEFs for small GTPase Ral (280), AF6/Canoe (281;282), RINl (283) and phophatidylinositol 3-kinase (P13K) (284). To date, Raf is the only Ras^{V12}C40 target protein for which genetic studies confirm its fundamental role in Ras, signaling in a normal cellular context. However, Ras^{v}12C40, an activated mutant of Ras with an alteration of tyrosine to cysteine at position 40 in the effector domain, is unable to bind Raf but is still able to cause tumorigenic transformation which demonstrates that Raf-independent pathways alone are sufficient to promote Ras transformation (285).

As overexpression of H-ras alone in primary cells would lead to senescence (286), malignant transformation of embryonic fibroblasts can only be achieved by the combination of H-ras with immortalizing oncogenes such as E IA or c-myc. By interacting with pRb (287) the adenovirus E1A protein promotes the dissociation of E2F from pRb (288;289) and induces the expression of S-phase specific genes. Another important cellular target of the Ad E1A protein is the transcriptional coactivator CBP/p300. CBP and its homologue p300 are large nuclear molecules that coordinate a variety of transcriptional pathways with chromatin remodeling. They interact with transcriptional activators as well as repressors, direct chromatin-mediated transcription, function in p53-mediated apoptosis, and participate in terminal differentiation of certain tissue types. The role of these proteins in human disease coupled with biochemical evidence suggests that CBP and p300 are tumor suppressor proteins essential in cell-cycle control, cellular differentiation and development (290). By targeting CBP/p300, Ad E1A thus can effectively abrogate p53 function and promote tumorigenesis.

The myc-family of proto-oncogenes includes three evolutionary conserved genes c-, N- and L-myc which encode related proteins (291, 292). After birth, proliferating tissues express c-myc in strict dependence on mitogenic signals. The c-myc protein is a transcription factor of the basic-helix-loop-helix-leucine zipper (bHLH-Zip) family. Myc must dimerize with another bHLH-Zip protein, Max, to bind the specific DNA sequence CACGTG (the E-box) and to activate transcription from adjacent promoters. Transcription-competent myc/Max dimers are the active form of myc in inducing cell cycle progression, apoptosis and malignant transformation. Myc as well as the viral protein E1A are “immortalizing” oncoproteins, which allow primary cells to bypass the senescence crisis and become established in culture. All these proteins cooperate with Ras, which is consistent with the fact that cellular immortalization is a prerequisite for full transformation by Ras. Oncogenic activation of myc genes results in their constitutive expression and contributes to progression of a wide range of human and animal neoplasias.

The observation that MIF deficiency in C57 B1/6 leads to accelerated growth in primary cells and reduced growth in transformed cells does not necessarily constitute a contradiction. Cell cycle progression and cellular transformation, although connected, are two different biological and experimental outcomes. This is exemplified best by the work of Alevizopoulos et al., who showed that activated Rb is ineffective in inducing G1 arrest in the presence of Myc while it was still effectively suppressing co-transformation by Myc and Ras (293).

It has been demonstrated that MIF stimulation of NIH3T3 fibroblasts led to a sustained activation of ERKI/2 and increased proliferation (71). Another potential target of MIF action was proposed by Kleemann et al. who identified MIF as binding partner and negative regulator of Jab-1 (79). Jab-1 is a coactivator of the transcription factor AP-1, which is composed of members of the Fos and Jun protein family and plays an important role in regulating growth and differentiation. AP-1 is well known as an important downstream effector of Ras. Its component c-jun has been implicated in the mechanism of transformation through several lines of evidence. First, c-Jun activity is induced by Ras activation. Second, c-Jun is oncogenic under certain conditions and, third, overexpression of dominant negative c-jun alleles can inhibit transformation by Ras proteins (294-297). The activity in a c-jun^{-/-} model shows an impaired ability for transformation by activated Ras proteins (298).

Besides AP-1 activation, Jab-I affects the cell cycle directly by stimulating the degradation of p27^{Kipl}, an inhibitor of cyclin-dependent kinases. MIF has been reported to stabilize p27^{Kipl} and to increase its expression levels (79). Although some evidence has been provided that p27^{Kipl} is able to act as activator of CDK4/cyclin D (299), it generally acts as inhibitor of CDK2/cyclin E and as tumor suppressor (300) which makes the MIF Jab1 interaction an unlikely candidate for the effect of MIF in transformation.

Another model of MIF's mechanism of action proposes that MIF inhibits the activity of p53 (106). MIF has been shown to rescue macrophages from p53-mediated apoptosis and to prolong the life span of MIFs in culture. p53 generally acts as an inducer of growth arrest and apoptosis in response as well as tumor suppressor in vivo (259). Therefore, MIF deficiency would be predicted to be associated with unbalanced p53 activity and reduced growth.

MIF may also be involved in tumorigenesis in vivo. Mounting scientific evidence suggests that MIF might be a tumor-promoting factor. Using differential display-PCR Meyer-Siegler et al. found increased expression of MIF in metastases of a prostatic adenocarcinoma (301). Overexpression of MIF in tumor tissue was also demonstrated in patients with hepatocellular carcinoma (302), lung adenocarcinoma (303) and glioblastoma multiforme (304).

Glucocorticoids are physiological hormones that are essential to life (77). They are synthesized in the adrenal cortex, secreted, and circulate at a concentration that fluctuates in a circadian rhythm (305). Glucocorticoids mediate many aspects of homeostasis and regulate the immune system as well as the stress response. Their powerful anti-inflammatory and immunosuppressive effects make them an extremely valuable therapy in patients with inflammatory disorders (306). Glucocorticoids internist the process of inflammation by inhibiting the activation of immune cells, by decreasing the production of inflammatory mediators or by inducing apoptosis of lymphocytes (307;308).

MIF has been described as a pro-inflammatory mediator which is released in response to glucocorticoids in vitro and in vivo and counteracts the glucocorticoid-induced suppression of cytokine production in macrophages and T-lymphocytes (16;69). Glucocorticoids in therapeutic doses lead to lymphocyte apoptosis in thymus and spleen, to atrophy of the adrenal cortex through suppression of ACTH secretion, hypogonadotropic hypogonadism and later to skin atrophy and muscular wasting by shifting the cellular metabolism to a catabolic state (220).

The temporal pattern of MIF induction correlates well with the sensitivity of these organs to glucocorticoids. Immune cells and cells of the adrenal cortex are sensitive to glucocorticoids whereas keratinocytes or muscle cells are much less so. This is reflected in the time-dependent appearance of the side effects of glucocorticoids: Suppression of the immune response or of the production of endogenous glucocorticoids is achieved quickly, whereas skin atrophy and muscular wasting occur later on. The transient nature of the changes in MIF expression may suggest that MIF might be involved in the cellular reprogramming during glucocorticoid stimulation.

The adrenal and the thymus provide clues to the MIF-glucocorticoid interaction. The adrenal gland is the main effector organ of the hypothalamic-pituitary adrenal axis (HPA-axis). Corticotropin releasing factor (CRF) from the hypothalamus stimulates the release of adrenocorticotropin (ACTH) from the anterior pituitary, which then stimulates the adrenal cortex to release glucocorticoids (309). This suggests that glucocorticoids positively regulate the expression of MIF in the adrenal cortex.

Thymic lymphocytes are very sensitive to supraphysiologic doses of glucocorticoids, which induce these cells to undergo apoptosis within several hours (310). Thymocytes undergo a sequence of maturation within the thymus during which they migrate from the cortex into the medulla and then enter the peripheral circulation (311). The more mature, medullary thymocyte is more resistant to glucocorticoid-induced death than the immature cortical thymocyte due to the expression of anti-apoptotic genes such as bcl-2 (312).

Recombinant MIF has been reported to inhibit nitric oxide (NO)-induced and p53-mediated apoptosis in murine macrophages (106), and dexamethasone has been shown to induce apoptosis in thymocytes in a p53-independent manner (95).

Glucocorticoids are anti-mitotic in several cell types and can repress positive regulators of the cell cycle or induce growth inhibitors depending on the cellular context. For example, inhibition of lymphoid cell proliferation, which partly accounts for the anti-inflammatory property of glucocorticoids, is mediated by a decrease in the levels of cyclin D and CDK4 (313;314) as well as c-myc (315). By contrast, in both hepatoma and lung alveolar cells, glucocorticoid-induced cell cycle arrest has been attributed to induction of the CKIp²¹Cip1 (316-318).

The glucocorticoid receptor has been reported to have the ability to function in several ways, mostly transcriptional, but also in some situations using posttranscriptional mode of actions, including stabilization of certain mRNAs. This mode of control is an important mechanism in the regulation of other CKIs. A posttranscriptional regulation of p27^{Kipl} was recently reported by Hengst et al (320).

### SUMMARY OF THE INVENTION

The present invention provides non-human animals, that can include all species of mammals other than human, but more particularly non-human primates, pigs, dogs, cats and rodents such as rats, and more particularly mice, that have been manipulated to be missing part or all, or essentially all of an activity of one or more specific gene/allele product(s).

In a preferred embodiment, the non-human animal has been manipulated so as not to express a fully functional MIF protein, or not to express any MIF protein.

In addition, the present invention provides cells, preferably animal cells, and more preferably mammalian cells that have been manipulated to be missing all or essentially all of an activity of the MIF protein. In a particular embodiment the mammalian cell is a murine cell. But it can also be a human cell, or other primate cell, as well as a cell of a pig, dog, cat or a rodent such as a rat, or mouse.

Another object of the invention is to produce an animal model to provide a genetic approach to further elucidate the biological function of MIF through development of a MIF knock-out mouse and MIF knock-in mice to study the contribution of MIF to cellular growth and development as well as to the host response to inflammatory and neoplastic disorders.

Yet another object of the invention is to construct a targeting vector that ensures complete loss of function when deleting the entire MIF gene (promoter and all exons) and normal expression of the MIF gene when flanked by loxP sites. One other object of the invention is to provide an animal model for expressing a mutant MIF protein.

Another object of the invention is to provide a method for preparing monoclonal or polyclonal antibodies to human MIF utilizing a transgenic mouse injected with human MIF, and the transgenic mouse has a genome that does not code for MIF, or codes for a MIF mutation. Yet still another object is to provide an in vivo method for selectively screening an inhibitor to a MIF activity not affected by a mutation to MIF.

### BRIEF DESCRIPTION OF THE DRAWINGS

FIG. 1A is a graphical representation of the design of the targeting vector. The targeting vector was constructed from 129/Sv genomic DNA wt allele, flanked the entire MIF-gene (5.5 Kb) by loxP-sites and putting the excisable neomycin-selection cassette into an inactive retrotransposon upstream of the MIF promoter. Legend is as follows:

=loxP site, =promoter, restriction sites: E=EcoR I, B=BamH I, X=Xba I, P=Pst I, Bg=Bgl II, S=Sal I, Sfi=Sfi I. Restriction sites in brackets ( ) were destroyed during cloning. neo=neomycin, tk=thymidine kinase.

FIG. 1B is a Southern blotting which shows identification of homologous recombinants by: EcoR I digest of genomic DNA from selected ES-clones was hybridized with an external, upstream probe A which gives a 10.5 Kb fragment for the wildtype allele and a 6.5 Kb fragment for the targeted allele. *=ES clone with homologous integration.

FIG. 1C demonstrates by Southern blotting integration of the third loxP site: A EcoR I digest of genomic DNA from homologous clones was hybridized with an external, downstream probe B which gives a 9.0 Kb fragment for the wildtype allele and a 8.4 Kb fragment for the targeted allele with cointegration of the third loxP site. +=ES clone with homologous integration and cointegration of the third loxP-site.

FIG. 2 is a Southern blotting showing results of testing for additional non-homologous integrants of the targeting vector Genomic DNA from ES cell clones with homologous or non-homologous integration of the targeting vector that was digested with BamH1, transferred to a membrane and hybridized with a neo-probe. Homologous clones show only a single band of the expected size 9.5 Kb, whereas non-homologous clones exhibit single or multiple bands of varying sizes.

FIG. 3 shows targeting by transient Cre-transfection followed by selection of floxed and deleted MIF. Homologous ES cell clones were transiently transfected with 3 μg of pPGK-Cre and screened for G418 sensitivity. Genomic DNA from G418-sensitive clones was extracted and tested for the presence of Neo and MIF by Southern blotting. EcoR I digest together with probe A yielded either a 10.5 Kb band (wildtype or floxed) or a 6.5 Kb band (knockout). Xba I digest together with probe C indicates the presence of floxed MIF when the 2.4 Kb fragment is obtained. E=EcoR I, X=Xba I, floxed=flanked by loxP sites. loxP site, =promoter.

FIG. 4 demonstrates genotyping by PCR in which genomic DNA from mouse tails having been extracted and analyzed by PCR using primers A, B and C. In case of the wildtype and the floxed alleles, amplification only occurs for primers B+C (544 bp or 683 bp). The knockout allele allows only the amplification of A+C (383 bp). The primers used are: A1: 5′-GAC GTG TAA CTC ATC GTC TCC-3′; B1: 5′-TTC AGC TGC AAG CGA TAC AGC-3′; and C1: 5′-GGC TAC GTA CCA GTT ACT TCG-3′. The reaction conditions for a 50 μl reaction are as following: 94 ° C.-2 min; 94° C.-1 min; 65° C.-45 sec; 72° C.-45 sec; 72° C.-10 min; 4° C.-forever; 32 cycles.

FIGS. 5A to 5C show validation of the MIF knockout mouse after male wildtype, heterozygote and homozygote mice were i.p.-injected with E coli LPS (15 mg/kg) and sacrificed 16 hours later. Genomic DNA, total RNA and protein were extracted from the livers.

FIG. 5A is a Southern blotting: EcoRI digest and hybridization with external probe A. The 10.5 Kb-fragment represents the wildtype allele, the 6.5 Kb-fragment the knockout allele.

FIG. 5B is a Northern blotting: mRNA specific for MIF and β-Actin (loading control) detected by specific cDNA probes.

FIG. 5C is a Western blotting: MIF and MAPK (p44/42) as loading control detected by the polyclonal anti-MIF antibodies R102 and anti-p44/42.

FIG. 6A is a graphical presentation of the mutational strategy for testing the catalytic base of the isomerase activity: The N-terminal Proline (Pro1) of exon 1 is changed to glycine by exchanging the codon CCT for GGC. The creation of the new restriction site for NcoI generates a restriction fragment length polymorphism (RFLP), which facilitates the detection of the mutation.

FIG. 6B is a graphical presentation of the mutational strategy for testing the CXXC-motif: Cysteine 60 of exon 2 is altered to serine by a point mutation in the codon TGC to TCC. to destroy the restriction site for PstI in the wildtype allele and create a novel restriction site for BpmI.

FIG. 7A is a graphical representation targeting strategy in the mutagenesis of MIF: H=Hind III, Sp=Spe I, E=EcoR I, B=BamH 1, S=Sal I. *=mutation.

FIG. 7B demonstrates screening for homologous recombinants by Southern blotting of BamH1-digested genomic DNA with external probe D. The 4.1 Kb fragment represents the wildtype, while the 3.3 Kb fragment represents the mutant allele.

FIG. 7C demonstrates the presence of the mutation in homologous ES-0cells: The PIG-mutation introduces a novel restriction site, which is detected by PCR and subsequent restriction digest with Nco I (novel 220/224 bp band). The C60S-mutation destroys a Pst I restriction site present in the wildtype allele. This is evident by PCR and subsequent Pst I digest which shows the presence of an undigested PCR-product (658 bp band).

FIGS. 8A to 8D validate the expression of mutant MIF by analysis of total RNA and protein was extracted from 8 week old male C57B1/6 mice with the genotypes wt/wt, wt/pg, pg/pg or cs/wt, cs/cs.

FIG. 8A is a Northern analysis with cDNA probes specific to MIF and β-Actin showing expression of MIFPG or MIFCS mRNA at equal levels with wildtype MIF.

FIG. 8B is a Western analysis from liver extracts with polyclonal anti-MIF (R102) demonstrating the presence of MIFpg and the absence of MIFcs.

FIG. 8C shows the results of a tissue screening for MIF protein expression in MIF^{CS/CS} and MIF^{wt/wt} mice.

FIG. 8D, in the upper panel shows tautomerase activity of recombinant murine MIF mutant human MIF^{plg} or MIF^{c60s} (140 μg/mL), while in the lower panel shows tautomerase activity in liver lysates from male, 8-12 week old MIF+/+, flox/flox, −/−, pg/pg and cs/cs mice (9000 μg/mL in RIPA buffer). L-Dopachrome methyl ester was used as substrate.

FIG. 9 is a sequence comparison of MIF^{c60s} versus MIF^{wt} from MIF^{cs60} cDNA cloned by PCR with primers ranging from the beginning of the 5′-untranslated region to the 3′-untranslated region just upstream of the polyA tail into pBSII-SK+. Sequencing was performed with T3/T7-primers flanking the insert and demonstrates that there are no additional mutations besides the cs6O mutation (TGC->TCC).

FIG. 10 is a graphical representation comparing a wild-type allele and the mutation allele.

### DETAILED DESCRIPTION OF THE INVENTION

According to the invention, a targeting vector was constructed that would ensure 100% loss of function in mice when deleting the entire MIF gene (promoter and all exons) and normal expression of the MIF gene when flanked by loxP sites. This was accomplished by first obtaining a MIF-containing P1-genomic clone from the mouse strain 129/Sv. The Neo-cassette for positive selection flanked by loxP sites was placed into an intracistemal A-particle (a type of retrotransposon) which is located upstream of the MIF promoter and has been described to be highly mutated and non-functional (33). A third loxP site was placed 1.2 Kb downstream of the MIF gene. Accordingly, loxP sites 2 and 3 flank a 5.5 Kb genomic fragment, which contains the MIF-gene.

A comparison of restriction fragments within the MIF locus in the mouse strains 129/Sv and C57MIF1/6 reveals no evidence for any restriction fragment length polymorphisms (RFLPs). Embryonic stem cells (ES-cells) from the strain C571/1/6 were the target. Targeted cells were selected by culture in G418 for 9 days and enriched for homologous recombinants using gancyclovir from day 5-7. Southern blotting of EcoRI-digested genomic DNA with an external, upstream probe identified several ES-clones that had an homologous integration of the vector and showed the expected 6.5 Kb fragment in addition to the 10.5 Kb wildtype allele. The overall frequency of homologous integration was 15%. The cointegration of the distant third loxP-site and the integrity of the downstream end of the MIF locus were verified by Southern blotting with the external probe B, which showed a double band of 8.4 Kb (targeted) and 9.0 Kb (wildtype) in 61% (24/39) of the homologous clones.

Design of Targeting Vector and Identification of Homologous ES-Clones

Design of the targeting vector: The targeting vector was constructed from 129/Sv genomic DNA, flanked the entire MIF-gene (5.5 Kb) by loxP-sites and put excisable neomycin-selection cassette into an inactive retrotransposon upstream of the MIF promoter.

Identification of homologous recombinants by Southern blotting: EcoR I digest of genomic DNA from selected ES-clones was hybridized with an external, upstream probe A which gives a 10.5 Kb fragment for the wildtype aliele and a 6.5 Kb fragment for the targeted allele.

Cointegration of the third loxP site: A EcoR I digest of genomic DNA from homologous clones was hybridized with an external, downstream probe B which gives a 9.0 Kb fragment for the wildtype allele and a 8.4 Kb fragment for the targeted allele with cointegration of the third loxP site

Additional non-homologous integration of the targeting vector did not occur in the genome of the selected homologous clones as evidenced by BamH1-digest of the genomic DNA and screening with a neo-specific cDNA probe by Southern blotting. All homologous clones showed the expected, single fragment of 9.5 Kb size, whereas the non-homologous clones showed one to several fragments of varying sizes.

Having successfully identified the ES cell clones with a single homologous recombination event and a cointegrated third loxP site, the next step was to remove the neomycin selection cassette, which was no longer necessary, and to obtain ES cell clones, in which MIF was either flanked by loxP sites (“floxed”) or deleted. This was performed by transiently transfected two homologous clones with a plasmid expressing Cre under control of the PGK-promoter (PGK=phosphoglycerat kinase, pPGK-Cre) ES cell clones that then became G418-sensitive (22/384 or 6%) were selected and analyzed by Southern blotting to determine whether Cre had excised the Neo selection cassette alone (i.e, allele with MIF flanked by loxP sites, MIF^{flox}) or the selection cassette and the MIF gene together (i.e., knockout allele, MIF). The knockout allele could be distinguished from the wildtype or the floxed allele by demonstrating the presence of the 6.5 Kb fragment after hybridization of EcoRI-digested genomic DNA with probe A. The distinction between the floxed and the wildtype allele was based on the presence of a 2.4 Kb fragment in XbaI-digested DNA detected with the internal probe C. This procedure yielded 3 ES cell clones, which carried a floxed MIF allele (3/22 or 14%), and 19 clones, which carried a knockout allele (19/22 or 86%). Then injecting the floxed and the knockout clones into BALB/c blastocysts to obtained several chimeric mice for each clone.

Male chimeras were bred to C57B1/6 females and pups that had inherited the targeted allele were identified based on their coat color (black=germline transmission, agouti=no germline transmission) and genotyping by PCR. Germline transmission was achieved only once for the knockout allele and five times for the floxed allele. Heterozygote pups were bred with each other to homozygosity. Due to the use of C57B1/6 ES cells, these mice are genetically pure C57B1/6.

Transient Cre-Transfection and Selection of Floxed and Deleted MIF

Homologous ES cell clones were transiently transfected with 3 μg of pPGK-Cre and screened for G418 sensitivity. Genomic DNA from G418-sensitive clones was extracted and tested for the presence of Neo and MIF by Southern blotting. EcoR I digest together with probe A yielded either a 10.5 Kb band (wildtype or floxed) or a 6.5 Kb band (knockout). Xba I digest together with probe C indicates the presence of noxed MIF when the 2.4 Kb fragment is obtained.

General Health, Weight, Mendelian Ratio of Inheritance and Fertility

The MIF^{-/+} and MIF^{-/-} mice were generally healthy, active and displayed no visible or histological organ abnormalities. The spontaneous death rate was low and comparable to the wildtype littermates. Littermates from heterozygote matings were weighed and genotyped at 6 weeks of age. In both males and females, the weight of the MIF^{-/+} mice was slightly higher than that of MIF^{+/+} or MIF^{-/-}, but the differences did not reach statistical significance. The weight of the MIF^{-/-} mice was comparable to that of MIF^{+/+} mice.

MIF is known to be expressed in reproductive organs such as the ovary and uterus of the female (232;233), and the testis and epididymis of the male (234;235). In order to determine the exact localization of MIF in the ovary, n situ-hybridization studies were performed which showed that MIF in the ovary was produced almost exclusively and in high quantities by granuloma cells (data not shown). These cells produced MIF throughout the stages of follicular development from primary to secondary and to mature follicles.

Based on the strong expression of MIF in the reproductive tract of males and females, it was determined whether MIF from these sources had an impact on function and survival of germ cells or embryos. This was resolved by mating 6-12 week old, heterozygote mice with each other and determined the distribution of the genotypes among the offspring by PCR typing of tail DNA. From a total of 319 pups the overall or sex-specific genotype distribution was essentially in agreement with Mendelian ratios (Table 1, below).

Validation of the Targeting Success

The effect of the targeting was evaluated on the levels of genomic DNA, mRNA and protein in livers of LPS-challenged MIF^{+/+}, MIF^{-/+} and MIF^{-/-} mice. Sixteen hours after an i.p. injection of a sublethal dose of E coli LPS (15 mg/kg), mice were sacrificed and the livers were processed to obtain genomic DNA, mRNA and protein. Southern blotting with probe A in EcoRI-digested genomic DNA confirmed the presence of a single 6.5 Kb band in the MIF^{-/-} mouse. Northern analysis with a full length MIF cDNA probe (exons 1-3) as well as Western blotting with the polyclonal antiMIF antibody R102 demonstrated the complete absence of MIF mRNA and protein in these animals.

TABLE 1
Inheritance of the MIF knockout allele

| | | |
|---|---|---|
| MIF^{+/+} | MIF^{-/+} | MIF^{-/-} |

All offspring:

| | | | | |
|---|---|---|---|---|
| Observed | 75 (24%) | 162 (50%) | 82 (26%) | 319 |
| expected | 79.75 (25%) | 159.5 (50%) | 79.75 (25%) | 319 |

| | |
|---|---|
| X^{2-test} | p = 0.8 = non significant |

Female offspring only:

| | | | | |
|---|---|---|---|---|
| Observed | 36 (20%) | 93 (53%) | 47 (27%) | 176 |
| expected | 44 (25%) | 88 (50%) | 44 (25%) | 176 |

| | |
|---|---|
| X^{2-test} | p = 0.4 = non significant |

Male offspring only:

| | | | | |
|---|---|---|---|---|
| Observed | 39 (27%) | 69 (49%) | 35 (24%) | 143 |
| expected | 35.75 (25%) | 71.5 (50%) | 35.75 (25%) | 143 |

| | |
|---|---|
| X^{2-test} | p = 0.9 = non significant |

These results demonstrate that MIF deficiency does not affect the function of germ cells or the survival of embryos under these laboratory conditions. MIF^{-/-} mice bred with each other appeared fertile with regards to litter size (6.5 pups per litter, 32 litters observed).

MIF Deficiency and the Response to Bacterial Lipopolysaccharide

Previous reports demonstrate inhibition of MIF by neutralizing monoclonal antibodies in BALB/c mice (21;63) as well as MIF deficiency in a genetically mixed 129Sv/C57B 16 background (236) confers protection from endotoxic shock. This protection is associated with reduced levels of TNF-a, but protection is also demonstrated in TNF-α knockout mice (63). However, this phenotype is not without contradiction.

One other independently created MIF knockout on 129Sv/C57B16 background had not demonstrated protection from LPS-lethality and the investigators had concluded that MIF deficiency does not influence LPS-induced cytokine levels or lethality (237). In order to clarify this issue, the response of MIF^{-/-} mice made according to the invention was determined towards LPS in vitro and in vivo.

Thioglycollate-elicited macrophages from MIF^{+/+} (n=3) and MIF^{-/-} (n=3) mice were harvested, purified by adherence and then stimulated with E. coli LPS (serotype 0111:B4). The supernatants were harvested at various time points and the kinetics of the release of the LPS-responsive mediators TNF-α, interleukin-6 (IL-6), prostaglandin E2 (PGE₂) were determined by ELISA (FIG. 38). Over a wide range of LPS concentrations (10 ng/mL-100 μg/mL) the LPS-elicited TNF-α or IL-6 response was not significantly different between MIF^{-/-} and MIF^{-/-} mice. The release of TNF-α was bell-shaped indicating that high concentrations of LPS were cytotoxic to macrophages. An analysis of the cell viability by the MTT assay indicated that LPS concentrations of 1-100 μg/mL are cytotoxic and reduce the viability of MIF^{+/+} as well as MIF^{-/-} cells by up to 25% (data not shown). Levels of IL-6 in the MIF-macrophage cultures were not different from controls. The kinetics of PGE₂ release showed that MIF^{-/-} macrophages were good producers of PGE₂.

As these initial in vitro studies did not reproduce the phenotype described by Bozza et al. (236), it was incumbent to study the MIF′-mice in the model of endotoxic shock. In two independent experiments male C57B1/6 MIF^{-/-} mice and their age- and sex-matched MIF^{+/+} littermates were injected with a LD₇₅ or a ₁₀₀ of E. coli LPS (37.5 mg/kg or 40 mg/kg) and monitored for their survival. Most of the deaths occurred between 15 and 30 hours after LPS-injection and there was neither a significant difference in the death rate nor in the final survival rates. As the outcome from lethal endotoxemia seemed to be independent of the presence or absence of MIF in this model, it was tested whether anti-MIF antibodies were still able to confer protection from LPS. MIF^{+/+} mice injected with either control-IgG1 (HB49, 200 μg/mouse) or neutralizing anti-MIF-IgG1 (XIV.15.5, 200pg/mouse) (63)) were slightly protected by anti-MIF therapy (30% survival versus 10% survival in controls, p<0.05). In another experiment the effect of anti-MIF was more pronounced (60% survival in anti-MIF-treated mice versus 20% survival in control-IgG treated mice, n=5 per group, p<0.05, data not shown). Anti-MIF therapy was specific to MEF as MIF^{-/-} mice were not protected by anti-MIF-IgG1 XIV. 15.5.

In order to test whether the genetic background had an influence on the LPS phenotype, it was also obtained MIF^{-/-} mice in which the MIF gene deletion had been bred to the BALB/c background for 6 generations. These MIF^{-/-} mice had been generated independently by the group of J. David at Harvard Medical School using a different targeting strategy and were shown to be LPS-resistant in endotoxentia experiments on a mixed 129/C57 background (236). When injected with a LD₁₀₀ (22.5 mg/kg) of E. coli LPS there was no detectable difference with regards to onset or kinetics of LPS-induced death in both age- and sexmatched groups.

These experiments suggest that deletion of MIF by gene targeting on C57B1/6 and BALB/c background does not protect mice from a lethal dose of endotoxin.

Effect of MIF Deficiency on Growth of Embryonic Fibroblasts

Several reports have demonstrated a link between MIF, growth control and tumorigenesis. MIF has been identified as an “delayed-early response gene” in NIH/3T3-fibroblasts (80) and its expression has been correlated with tissue development and differentiation (13). Furthermore, MIF has been implicated in the regulation of growth and cell cycle regulatory proteins such as MAPK (71), p53 (106), Jabl-API (79). Increased expression of MIF has been observed in several human malignancies and has been linked to a more aggressive tumor phenotype (238;239).

Murine embryonic fibroblasts (MEFs) are a standard model to study growth properties and cell cycle control in primary cells as they are easy to obtain and grow as monolayer in vitro for a certain number of passages. In order to study the influence of MIF deficiency on growth properties and cell cycle control, murine embryonic fibroblasts (MEFs) were prepared from MIF^{-/-} and MIF^{+/+} embryos at day 14.5 of embryonic development from the strains that were available:

1) C57B1/6

2) BALB/c (F6 of Harvard knockout),

3) 129Sv/C57B16 (F3 of Harvard knockout)

These MEFs were cultured in vitro for 10 passages under conditions of high and low cell density. Under high density conditions (30,000 cells/CM²) MIF^{-/-} MEFs from C57B1/6 and BALB/c proliferated more rapidly than the respective MIF^{+/+} controls. Expressed in terms of cell divisions, the MIF^{-/-} MEFs divided twice as rapidly as MIF^{+/+} MEFs over a period of 30 days (average doubling times: C57B1/6: MIF^{+/+} 4 days versus MIF^{-/-} 2 days; BALB/c: MIF^{+/+} 6 days versus MIF^{-/-} 3 days). The genetic background demonstrates to be of importance as MIF^{-/-} MEFs from 129Sv/C57 (F3) showed the reverse phenotype and proliferated more slowly than wildtype controls). Staining with Trypanblue did not reveal a significant difference in cell death between both genotypes. The same result was obtained when examining DNA synthesis: After synchronization by serum starvation for 72 hours, more MIF^{-/-} cells successfully completed the G1/S transition and replicated their DNA as evidenced by a 2-fold increase in ³H-thymidine uptake compared to controls. The duration of the G1/S-transition was not different from MIF^{+/+} cells suggesting that MIF^{+/+} cells do not cycle faster than MIF^{+/+} cells.

Several genes that regulate the cell cycle such as mitogen-activated protein kinases (MAPK, ERK1/2), cyclin E, cyclin A, cdc6, B-myb are known to be induced or activated during G 1/S transition. Others such as the CDK inhibitor p27^{Kipl} are downregulated when the cells start to proliferate (88;89). In order to test whether the differences in growth between MIF^{-/-} and MIF^{+/+} MEFs is due to differences in the expression of these genes, stimulation of synchronized C57B1/6 MIF^{-/-} and MIF^{+/+} fibroblasts was done with 10% serum and analysis of expression of activated MAPK, cyclin E, cyclin A, cdc6, Bmyb and p27^{Kipl} by Western blotting was done in the first 16 hours after serum addition. Levels of phospho-MAPK were elevated to a slightly higher degree in MIF^{-*-} fibroblasts, but otherwise we found no alteration in the MIF^{-/-} cells compared to wildtype controls.

MEFs grow to form a monolayer and then arrest due to contact inhibition by neighboring cells. The phenotype of accelerated growth in the MIF MIF^{-/-} MEFs was not present under conditions of low density (4000 cells/cm²) shows that a certain degree of contact inhibition by neighboring cells is required to manifest the phenotype. To further test the influence of MIF deficiency on contact inhibition of growth, MEFs were plated from C57B1/6 or BALB/c at high density and cultured to confluency. Five days after the MEFs had reached confluency the total cell number was determined. In both strains, MIF^{-/-} MEFs reached a 30% lower density than MIF^{+/+} counterparts. This could not be explained by differences in cell size, as MIF^{-/-} MEFs were identical in size to MIF^{+/+} controls when assessed by flow cytometry, nor by differences in the amount of dead cells (data not shown). This suggests that MIF^{-/-} cells become contact inhibited at a lower density than MIF^{+/+} cells.

Animal models according to the invention provide a novel approach to analyze the effect on cell growth and cell cycle regulation in genetically engineered MIF-deficient embryonic fibroblasts. MIF-deficient murine embryonic fibroblasts (MEFS) have been derived from two different gene targeting approaches on three different genetic backgrounds (C57B1/6, BALB/c and 129/Sv). Both targeting approaches provide similar observations that MIF-deficiency causes altered growth properties of fibroblasts. Specifically, in C57B1/6 and BALB/c MIF^{-/-}, MEFs proliferate faster under high-density conditions and a higher percentage of cells successfully complete the GIS transition and enter S-phase. In addition, MIF^{-/-} MEFs become contact inhibited at a 30% lower density compared to wildtype cells. These results strongly suggest that MIF acts as a regulator of the cell cycle and replication.

The growth phenotype of 129/Sv MIF^{-/-} MEFs is the opposite of MEFs on BALB/c or C57B1/6 background, that is, this suggests the existence of strain-specific modifier loci, a finding which has also been reported for targeted mutations in p107 (266), p130 (262), IGF-1 (267), fibronectin (268). EGF (269;270), cystic fibrosis transmembrane conductance regulator (CFTR) (271), TGFβ1 (272), TGFβ3 (273) and the β1-adrenegic receptor (274). Loss of E2F 1 reduces a previously reported strain-dependent difference in Rb1 (+/−) lifespan, suggesting that E2F 1 or an E2F 1-regulated gene acts as a genetic modifier between the 129/Sv and C57BI−/6 strains (275). Another example is p16^{INKa}, which has been shown to be less active in BALB/c compared to DBA/2 mice. This reduced efficiency of the p16^{INKa}, allele in BALB/c has been identified as being the cause for the susceptibility of BALB/c mice to plasmocytoma (276).

MIF Deficiency in Ras-Mediated Oncogenic Transformation of Fibroblasts

The features of MIF deficiency that lead to altered growth properties make it interesting from the perspective of tumorigenesis. Growth and malignant transformation are inter-related processes, which often involve the same regulating pathways and molecules. MEFs are a standard model to study the process of immortalization and transformation. The goal was therefore to investigate the role of MIF in the malignant transformation of MEFs using the focus formation assay as readout. This was accomplished by using replication-defective retroviruses produced from the retroviral vector REBNA (240) to immortalize primary C57B1/6 MEFs with either the viral oncoprotein E1A or c-myc and which subsequently transformed them by additional transfer of oncogenic Ras (H-ras). The presence and expression of these oncoproteins at roughly identical levels was demonstrated by Western blotting of cell lysates. While levels of E1A expression achieved were slightly higher in MIF^{-/-} cells, the levels of c-Myc expression were slightly lower.

When these cells were cultured in low number (n=1000) on a monolayer of primary cells (n>300,000), the transformed cells grew to visible colonies within 10-14 days. E1A+H-ras transformed MIF^{+/+} cells on MIF^{-/-} feeders readily formed colonies that were spreading and expanding, but MIF^{-/-} cells on MIF^{+/+} feeders produced only half the number of colonies and these colonies were much smaller in size (MIF^{+/+} 214±5 versus MIF^{+/+} 117±5, p<0.001).

Colonies formed by c-myc+H-ras-transformed MEFs were generally less compact and more widespread than colonies formed by EIA+H-ras-transformed cells. In this case, MIF^{+/+} cells were observed to begin colony formation, but then underwent regressive changes that resembled apoptosis and only a few scattered transformed cells remained which were hardly detectable. The number of colonies produced after 14 days was greatly reduced in MIF^{-/-} cells compared to MIF^{+/+} controls (MIF^{-/-}69±2 colonies versus MIF^{+/+} 2±1 colonies, p<0.002). This demonstrates that MIF is necessary for efficient Ras-mediated oncogenic transformation.

MIF has been described as an autocrine/paracrine growth factor. Therefore, testing was done to determine whether the difference in the proliferative capacity of the MIF^{+/+} and MIF^{-/-} transformed cells was dependent on the presence of a feeder layer and/or on the presence of MIF in the media. In the absence of a feeder layer, E1 A/H-ras transformed MIF^{-/-} cells grew at a slower rate compared to MIF^{+/+} cells showing that the defect in proliferation is at least partly attributable to the transformed cells themselves.

It was also determined whether the growth rate of the transformed cells was dependent on the presence or absence of MIF from the media. As expected, the growth of MIF^{+/+} cells was not influenced by the absence or presence of MIF in the media, as these cells are able to produce the MIF they require. Although fibroblast-conditioned media provided some growth stimulus to MIF^{-/-} cells, this effect was independent of the absence or presence of MIF. These results indicate that the growth rate of transformed MIFs is independent from the extracellular levels of MIF and that the growth deficiency of MIFf−/− transformed cells is due to a defect in the intracellular machinery of the cell.

Culturing E1A+H-ras transformed MIF^{+/+} or MIF^{-/-} MEFs on either a MIF^{+/+} or MIF^{-/-} feeder layers did reveal that the feeder layer and the transformed cells may influence each other. An MIF^{-/-} feeder layer decreased the number of colonies in both MIF^{+/+} and MIF^{-/-} -transformed cells (in MIF^{+/+} transformed cell: −10%, in MIF^{-/-} cells: −30%). These findings demonstrate that MIF deficiency affects the cell-to-cell interaction.

In summary, the analysis of MIF knockout cells demonstrates that MIF functions as a regulator of growth and cell cycle in embryonic fibroblasts. MIF^{-/-} fibroblasts differ from MIF^{+/+} fibroblasts in several characteristics such as growth over a long period of time, entry into S-phase of the cell cycle after serum starvation, and contact inhibition.

It was demonstrated that the phenotype of MIF deficiency is strongly influenced by the genetic background of the mouse strain. Furthermore, MIF seems to participate in the process of Ras-mediated malignant transformation of fibroblasts. As it is well established that ras is an important proto-oncogene, which is frequently mutated in mammalian cancers (241). This provides further evidence that MIF may participate in tumorigenesis.

Mutagenesis of MIF

Genetic analysis allows not only the full inactivation of genes by knockout targeting, but offers also the possibility to test hypotheses or predictions that arise from other means of investigation such as structural biology or biochemistry. One of the intriguing aspects of MIF's biology was the finding that MIF belonged to a novel protein superfamily that was subsequently shown to include enzymes. The members of this family share the barrel-like structure, are composed of identical subunits (either homotrimer or homohexamer) and exhibit enzymatic function as isomerases. In the case of MIF, the N-terminal proline (proline-1) had been identified as the catalytic base responsible for isomerase activity and lies at the base of the “catalytic pocket” of MIF (138).

Other known structural components of MIF include a CXXC-motif in position 57-60, which may be relevant for protein folding or protein-protein interactions (151). By mutagenizing proline 1 and Cys60 in vivo it was possible to test the biological relevance of the proposed isomerase activity and of the CXXC-motif.

Choice of the Mutational Strategy

The design of the mutagenesis of the N-terminal proline was guided by the idea that the mutation should disrupt only the enzymatic activity, and only minimally perturb the structure of the conserved pocket, which might be the binding site for an interacting protein. Several recombinant proteins with mutations in the N-terminus have been created. Replacing proline by amino acids with aliphatic side (e.g. alanine) or aromatic chains (e.g. phenylalanine) leads to a complete loss of isomerase activity (144). However, these mutations affect the structure of the catalytic pocket by their side chains. Moreover, they lead to the retention of the initiator methionine, which is cleaved off in the wildtype protein. Only the mutation of proline to the smallest amino acid glycine has been shown by X ray crystallography to preserve the structure of the pocket while eliminating the isomerase activity of MIF (141). The N-terminal proline is encoded by the codon CCT in the mouse. According to codon usage in the mouse the codon GGC was a frequently used codon for glycine (242). Replacing the triplett CCT by GGC also created a novel restriction site for the enzyme NcoI.

The efficiency of the P>G mutation in destroying the isomerase activity was verified first in recombinant mutant proteins in the tautomerase assay with dopachrome methylester as substrate. The P>G protein had no detectable activity. Cysteine 60 was mutated by a single point mutation (G>C) to serine, another polar amino acid. This mutation destroyed the original PstI restriction site and created a new restriction site for BpmI on the opposite strand.

Mutagenesis of the MIF by Gene Targeting

When intentionally introducing a specific mutation by a knock-in approach, it is necessary to co-introduce a selection cassette in order to identify the transfected cells out of millions of untransfected cells. As selection cassettes may interfere with gene expression or gene regulation, this might be the cause of artificial phenotypes in the mutant mouse. Therefore, a Cre-containing selection cassette was chosen as it would delete itself after identification of the correct ES-cell clones, leaving only a single loxP-site in the genome. Such a cassette (pACN) was constructed by M. Bunting et al. (243) and provided by M. Capecchi and K. Thomas (University of Utah, Salt Lake City). pACN contains the selectable marker Neo driven by the polymerase H promoter as well as the Cre recombinase under control of the testis-specific tACE promoter (tACE=testis-specific angiotensin converting enzyme). The two genes are flanked by loxP sites, which subsequently allow the self excision of the cassette by Cre when the ES-cell undergoes differentiation to sperm cells. Thus, offspring with germline transmission of the targeted allele from the chimeric mice will inherit the mutation and a loxP site.

Engineering each of the mutations separately in a 2 Kb MIF-containing SpeI-DNA fragment by PCR mutagenesis allowed sequencing of the entire insert to make certain that the desired mutation was present and that the PCR had not introduced any additional random mutation. The ACN-cassette was placed downstream of the MIF-polyA and thymidine kinase was included at the 3′-end of the targeting vector. As in the MIF knockout, C57B 1/6 ES-cells (Bruce-4) were transfected to obtain several homologous recombinants for each construct, which showed the expected additional 3.3 Kb band in BamHI-digested genomic DNA hybridized with the external probe D. The presence of the mutation in the homologous ES-cells was confirmed in two ways: by PCR and subsequent restriction digest as outlined in Figure mutational strategy as well as by direct sequencing of the mutation-containing PCR fragments.

Exclusion of the presence of additional vector integrants was done by HindIII-digest and hybridization with a neo-probe and then injecting one ES-clone for each mutation into BALB/c blastocysts. Several male chimeras transmitted the mutant allele to some of their offspring and these heterozygous mice were bred to homozygosity. The Cre-mediated excision of the selection cassette pACN was complete in 100% of the germline events as assessed by PCR (data not shown).

Expression of Mutant MIF

In order to investigate that potential phenotypes in the mutant mice were not due to insufficient expression of mutant MIF, MIF expression levels in male wildtype, heterozygous and homozygous animals was tested by Northern and Western blottings. MF^{PG} mRNA was express at levels equivalent to wildtype MIF; the MIF^{PG} protein was slightly reduced. However, whereas MIF^{CS} mRNA was expressed normally and the polyclonal anti-MIF antibodies detected recombinant MIF^{cs60} from E. coli, MIFCS protein was not detectable in homozygous mice even in a variety of tissues such as muscle, skin, liver, spleen and kidney.

In order to exclude the possibility that an additional mutation in the MIF^{CS60} gene might be causing a premature stop of translation of a frameshift, the MIF^{cs60} mRNA was cloned from the beginning of the 5′-untranslated region to the beginning of the polyA tail from spleen of a MIFcs/cs mouse. Sequencing showed that there was no mutation, which could account for the loss of protein expression besides the cs6O-mutation.

The Phenotype of the Mutant Mice

Mice with the genotype MIF^{pg/pg} or MIF^{cs/cs} appeared normal and fertile and arose from heterozygous intercrosses at a predicted Mendelian frequency (71:102:59; wt/wt:pg/wt:pg/pg) (51:101:44; wt/wt:cs/wt:cs/cs). A histological survey of skin, spleen, liver, kidney, heart, lung, skeletal muscle, intestine, ovary, adrenal and brain in 8-week old female homozygous mice did not reveal morphological abnormalities.

In order to test the biological impact of these mutations, homozygous (pg/pg or cs/cs) embryonic fibroblasts were prepared at day 14.5 and studied their behavior in E1A/H-ras-mediated oncogenic transformation in parallel with MIF^{+/+} and MIF^{-/-} fibroblasts. MEFs were first immortalized by infection with a retroviral E1A construct and then transduced with retroviral oncogenic Ras (H-ras). The resulting levels of expression of E1A and H-ras were comparable between the strains. 1000 transformed cells were cultured with 300,000 untransformed cells for 14 days in the focus formation assay. Transformed pg/pg MEFs produced approximately equivalent numbers of colonies as MIF^{+/+} when cultured on the genotypically identical feeder layer (pg/pg on pg/pg 196±15, +/+on+/+209 +8, p=n.s.). The size of the colonies was dependent on the feeder layer and were very small on MIF′-feeders.

The number of colonies in cs/cs-MEFs equaled the number of colonies in −/−MEFs (cs/cs 105±14, MIF^{-/-} III±12) and was significantly reduced from MIF^{+/+} which is well in keeping with the lack of protein in these cells. Again, the number and size of the colonies were reduced on −/− feeders and restored on +/+ feeders. In two out of four experiments, cs/cs-feeder still seemed to increase the colony size of cs/cs transformed cells. This effect may reflect some low-level expression of MIF^{cs60}.

The similarity of the pg/pg-MEFs to the MIF^{+/+} fibroblasts leads to the conclusion that the pg-mutation does not generate a noticeable loss of MIF function in H-ras-mediated oncogenic transformation of fibroblasts. Therefore, the isomerase activity of MIF is not likely to be MIF's underlying mechanism of action with respect to this activity. The resemblance of the MIFcs/cs MEFs to the MIF^{-/-} fibroblasts appears to be due to the loss of MIF protein expression, which makes it difficult to study the biological effect of the cs-mutation in vivo. Cysteines often fulfill important functions for the folding and structure of proteins. Disruption of these structural components often leads to misfolded proteins, which are rapidly targeted for destruction by the ubiquitin-proteasome pathway (244).

### Material and Methods

Western Blotting

For the detection of MIF protein, mouse or rat tissues were homogenized in REPA-buffer with a rotor-stator-homogenizer. Lysates from cultured cells were prepared by adding 1×PBS/1%Tween 20 to the pellet, incubating for 30 min on ice with frequent vortexing. Then the remaining fragments were pellet by centrifugation at 10,000 g for 30 min and the supernatant frozen at −20° C.

After determination of the protein concentration with the BioRad DC (BioRad) equal amounts of protein were heat denatured, separated on a 18% SDS-polyacrylamide gel, and blotted to nitrocellulose (Immobilon, Millipore, Bedford, Mass.). Blots were blocked with blocking buffer containing horse serum, probed with antibodies that recognize murine MIF followed by a horseradish peroxidase-conjugated donkey-anti-mouse antibody, and detected using enhanced chemiluminescence (ECL, Amersham Pharmacia).

For the detection of cell cycle proteins in murine embryonic fibroblasts, we used antibodies against P44/42 MAPK, phospho-p44/42 MAPK, cyclin E and A, B-myb, E1A, c-myc, H-ras and p27^{Kipl} from Cell Signaling Technology, Inc. (Beverly, Mass.).

Immunohistochemistry

The paraformaldehyde-fixed and paraffin-embedded tissues were cut into 5- to 6-gm sections, mounted onto poly-L-lysine-coated glass slides, deparaffinized in xylene, and passed through decreasing concentrations of alcohol in water. The specimens were then treated in 3% H₂O₂ in PBS for 30 min in the dark to inactivate endogenous peroxidases. The sections were then incubated in blocking solution (LSAB horseradish peroxidase kit, DAKO, Botany, Australia) for 30 min and stained with the monoclonal anti-MIF antibody III.D.9 ON at 4° C. IgG1 isotype control was used as negative control. After three washes in 1×PBS/0.05% Tween 20, the bound antibody was visualized with the DAKO LSAB horseradish peroxidase kit. The sections were stained with 3-amino-9ethylcarbazole as chromogenic substrate and counterstained with Meyer's hematoxylin.

In Situ-Hybridization

A MIF probe was prepared by subcloning the 420 bp XbaI/BanH1 cDNA fragment from pET11b into the Bluescript SK+ vector (Stratagene, La Jolla, Calif.). The plasmid was linearized for the generation of MIF sense and antisense riboprobes. Both probes were labeled with [³³⁵]dUTP and in situ hybridization on formalin fixed tissue sections was performed by Molecular Histology Inc. (Gaithersburg, Md.).

Mouse Endotoxemia Model

Male 8-12 week old MIF^{-/-} C57B1/6 mice and their MIF^{+/+} littermates were obtained from Charles River Laboratories (Wilmington, Mass.). After a minimal recovery period of 5 days, they were injected intraperitoneally with 15-35 mg/kg of E. coli LPS (serotype 0111:B4, Sigma) at 10 a.m. Survival was monitored every 4 hours during daytime and the time and number of deaths recorded.

DNA Sequencing

The DNA sequence of plasmids was determined using the Big Dye Terminator Cycle Sequencing Kit from Perkin Elmer (Foster City, Calif.). Briefly, 0.8-1.0 μg of DNA were amplified with 3.2 pmole of primer and terminator ready reaction mix in a total volume of 10 μl. The products were purified from unincorporated nucleotides using Centri-Sep spin columns (Princeton Separations, Adelphia, N.J.) or ethanol precipitation. The reaction was analyzed by the sequencing department of North Shore University Hospital (NSUH).

Cloning of Oligonucleotide Adapters

For adapter cloning, 5′-phosphorylated oligonucleotides coding for the sense- and antisense-strand were purchased from GibcoBRL (Grand Island, N.Y.) and resuspended in water to a concentration of 200 μM. To prepare the adapter 100 mmoles of each oligonucleotide were combined in 1 ml of 1×annealing buffer (100 mM Tris-HCl pH 7.5, 1M NaCl, 10 mM EDTA in nuclease-free water). To properly anneal the oligonucleotides, they were boiled at 94° C. for 5 min and then allowed to cool slowly at room temperature for 2 hours. The adapter was then subcloned into the vector using the TaKara Ligation Kit (TaKara/PanVera, Madison, Wis.) with a 100-fold excess of adapter over vector.

Preparation of Competent Cre-Bacteria

Frozen bacteria carrying Cre were obtained from K. Rajewsky, Cologne, Germany. A frozen vial was thawed, streaked on an agar plate without antibiotic and cultured at 37° C. ON. 250 ml of SOB medium were inoculated with 10-12 large colonies in a 2 litre Erlenmayer flask and grown at room temperature under continuous stirring to an A₆₀₀≈0.6. The bacteria were then washed once in ice-cold TB-buffer, pelleted and resuspended in 20 ml of TB. DMSO was added slowly to a final concentration of 7%. After 10 min incubation at 4° C. the bacteria were aliquoted, shock frozen in liquid nitrogen and stored at −70° C.

Bacterial Transformation and Plasmid Preparation

In order to obtain more of the individual plasmids each plasmid was incubated with 5 OW of DH5a bacteria (subcloning efficiency) from GibcoBRL for 20 min on ice and then subjected to heat shock (40 seconds at 42° C.). This mixture was spread on an agar plate containing ampicillin (100 μg/ml) and incubated ON at 37 ° C. The next morning 6 ml of ampicillin-containing LB broth were inoculated with a single bacterial colony and grown to end log phase over 24 hours at 37° C. on a platform shaker.

For a plasmid miniprep, 3 ml were used to extract the plasmid using the Wizard Plus Minipreps DNA Purification System (Promega, Madison, Wis.). For a plasmid maxiprep, 100-250 ml of ampicillin-containing LB broth were inoculated with 10 μl of bacterial suspension and grown to mid-log phase at 37° C. on a platform shaker. The plasmid was extracted and purified with the Qiagen Maxiprep Kit (Qiagen, Valencia, Calif.).

Three P1 plasmids containing the MIF gene were purchased from Genome Systems (now Incyte, Palo Alto, Calif.). In order to purify the P1 plasmid the plasmid had first to be transferred from the Cre+host NS3529 to the Cre-host NS3516 via transduction. The P1 clone was grown ON in L-broth and the pellet of 1 ml of culture was resuspended in L-broth containing 5 mM CaCl₂ 1×10⁹ of Plvir phage was added to the bacteria and allowed to adhere for 5 min at 37° C. Then the cells were pellet again, resuspended in 1 ml of L-broth containing kanamycin (25 μg/ml) and 10 mM MgCl₂ and the phage allowed to infect the bacteria for 2 hours at 37° C. on a platform shaker. The transducing phage was extracted from the bacteria by adding 2 μl of chloroform and vigorous vortexing for 30 seconds. After pelleting the cellular debris and chloroform by centrifugation the supernatant which contained the active transducing phage was stored at 4° C.

The P1 clone was transduced into the Cre-bacteria NS3516 by mixing fresh transducing phage with freshly grown NS3516 bacteria, 5 min adsorption and 45 min infection in L-broth+10 mM sodium citrate. Then the bacteria were spread on L-agar plates containing kanamycin for selection of P1+bacteria and grown ON at 37° C. The next day one P1+colony of NS3516 was grown in L-broth+kanamycin ON to stationary phase. Then 75 ml of L-broth+kanamycin were seeded with 2.5 ml of the miniprep for 1.5 hours and induced by IPTG (0.5 mM) for another 5 hours. Then the bacteria were harvested, lysed using lysozyme (Ready Lyse, Epicentre Technologies, Madison, Wis.) and alkaline lysis. Protein was precipitated by acidification and the P1 plasmid extracted by the phenol/chloroform method.

Preparation of γ-Irradiated Embryonic Feeder Cells (EF-Cells)

Preparation of EF-cells required sacrifice of two approximately 14 day-pregnant mothers from the DR4-strain and dissected their embryos. The embryos were washed 3 times in 1×PBS and the organs liver, spleen and heart as well as the head were removed. The remaining embryonic body was dissected into small pieces and trypsinized to single cell suspension in 50 ml of Trypsin/EDTA stirred at 37° C. in the presence of sterilized glass beads. This digest was stopped after 30 min by adding 50 ml of culture medium. The remaining cell aggregates were removed by filtering through a sieve. 3×10⁶ EF-cells were then plated in 15 cm-Petri dishes and grown to confluency for 3 days. Cells were then harvested using Trypsin/EDTA, γ-irradiated with 3000 rad in a Gammacell 1000 Blood Irradiator (Atomic Energy of Canada Ltd., Mississauga, Ontario, Canada), frozen in 10% DMSO/40% FCS and stored in liquid nitrogen.

Culture/Transfection of Bruce4 Embryonic Stem Cells with the Targeting Vector

The Bruce4 embryonic stem cells (ES-cells) were obtained from K. Rajewsky, University of Cologne, Germany. The ES-cells were grown in gelatin-coated plates at 37° C. and 10% CO₂ on a layer of y-irradiated, G418-resistant embryonic fibroblasts (“feeder cells”) which were prepared from embryos of the DR4-mouse strain. The culture medium consisted of DMEM with L-glutan-tine (GibcoBRL) supplemented with 15% heat-inactivated FCS, sodium-pyruvate, non-essential aminoacids, P-mercaptoethanol and leukaemia inhibitory factor (LEF) according to manufacturer's instructions. The LIF was from supernatant of LIF-transfected CHO cells (Genetics Institute, Cambridge, Mass.) and was obtained through K. Rajewsky.

The FCS used was purchased from Boehringer Mannheim (Lot 148269-02, now Roche, Indianapolis, Ind.) after it had been tested that it did not promote ES cell differentiation. As a rule, ES cells were fed every 24 hours and always 2-4 hours before any handling was performed. Generally, the Bruce4-cells were trypsinized using Trypsin/EDTA containing 2% heat-inactivated chicken serum (Sigma, St. Louis, Mo.)).

For the transfection experiments, Bruce4-ES cells were expanded and 1×10⁷ cells transfected in transfection buffer (20 mM HEPES, 137 mM NaCl, 5 mM KCl, 0.7 mm Na2HP04, 6 mm glucose, 0.1 mm β-mercaptoethanol) with 30 μg of linearized targeting vector using a BioRad Gene Pulser (230 V, 500 μF capacitance, 3 seconds) (BioRad, Hercules, Calif.). After the transfection, cells were incubated for 5 min at room temperature, then resuspended in ES media and plated onto gelatinized feeder plates. 48 hours after the transfection, I started the positive selection with G418 (Gibco BRL) in an active concentration of 163 μg/ml. From day 5 to day 7 after transfection, the negative selection using gancyclovir (Cymeven, Syntex/Roche, Indianapolis, Ind.) in a final concentration of 2 μM was added. On days 9 and 11 after transfection, I picked single, undifferentiated colonies and divided them onto two 96-well feeder plates. After this the clones on the plate for long-term storage were grown to subconfluency, washed twice with 1×PBS, trypsinized to a single cell suspension and frozen in 96 well plates in 10% DMSO/40% FCS at −70° C.

Neo-Deletion of Homologous ES-Clones by Transient Cre-Transfection

For the deletion of the selectable cassette in the previously identified homologous ES-clones, these clones were first expanded and two of them then transfected in a similar fashion as before this time using 3 μg of circular plasmid containing Cre under control of the phosphoglyceratkinase promoter (PGK-Cre, gift from K.Rajewsky) per 1×10⁷ cells and in the absence of G418. After transfection the cells were grown for another 48 hours without G418, then expanded for some more days without G418. On day 8 after transfection, 300 clones were picked and split 1:2 onto two 96-well plates. One plate served as master plate and was not treated with G418. The other plate was a duplicate and was treated with G418 in a concentration of 326 μg/ml (active form). After 2-3 days of G418 selection, all Neo-deleted clones were dying and could be clearly differentiated from the Neo-containing clones. The corresponding clones were harvested from the master plate and expanded for freezing and preparation of genomic DNA.

Restriction Digest of ES Cell Clones for Southern Screening

The plate for Southern analysis was grown to confluency, washed twice with 1×PBS and the cells were lysed in 50 μl of lysis buffer (16 mm NaCl, 10 mm Tris-HCl pH 7.5, 10 mm EDTA, 0.5% Sarcosyl, 0.4 mg/ml proteinase K) at 55° C. in a humidified atmosphere ON. After cooling to room temperature, the genomic DNA was precipitated by addition of 100 μg of 100% ethanol. The plate was washed further 3 times with 70% ethanol, airdried and redissolved in 35 μl of 1 × restriction digest (1× restriction buffer, 1 mM spermidine, 1 mM DTT, 100 pg/ml BSA, 50 μl g/ml RNase, 50 units of restriction enzyme). The restriction digest was done ON at the appropriate temperature and the prepared DNA prepared for Southern blotting the next day.

Blastocyst Injection

The injection of Bruce4 ES-cells into BALB/c-blastocysts was done at the gene targeting facility at Cold Spring Harbor Laboratories, NY. Briefly, female BALB/c mice were superovulated with PMS (pregnant mare serum, 10 units i.p. 4 hours before darkness) and HCG (human choriogonadotropin, 10 units i.p. 48 hours after PMS) and then mated with a fertile BALB/c male. Blastocysts were harvested and each injected with 15-18 targeted Bruce4-ES-cells. The injected blastocysts were then implanted into the oviduct of pseudopregnant foster mothers and developed into embryos.

Preparation of Genomic DNA From Mouse Tissue

Genomic DNA from mouse organs was prepared according the protocol 2.2.1 in Current Protocols in Molecular Biology. The tissue was excised, immediately frozen in liquid nitrogen and stored at −70° C. until use. Then 1 g of tissue was ground to a fine powder using a prechilled mortar and pestle and digested in digestion buffer (100 MM NaCl, 10 mm Tris HCl pH 8.0, 25 mm EDTA pH 8.0, 0.5% SDS, 0.1 mg/ml proteinase K) ON at 55° C. in a thermocycler (Eppendorff/Brinkmann Instruments, Westbury, N.Y.). After digestion, the sample was extracted with phenol/chloroform/isoamylalcohol, precipitated and washed with 100% or 70% ethanol, air dried and resuspended in TE-buffer.

Breeding and Genotyping of Mice

Mice were bred at Charles River Laboratories (Wilmington, Mass.) or at North Shore University (Manhasset, N.Y.). Mouse tails (0.4-0.6 cm) from 3-4 wk old mice were cut into an 1.5 ml Eppendorff-tube and stored at −70° C. until use. The genomic DNA was then extracted using the Dneasy Tissue Kit (Qiagen), eluted in 200 μl of TE-buffer (10 mm Tris HCl, 1 mm EDTA, pH 8.0) and stored at 4° C.

For genotyping by PCR, typically 1 μl of genomic DNA was amplified with specific primers and PCR Supermix (GibcoBRL) in a 50 μl reaction (annealing 65 ° C., 35 cycles). The PCR product was then loaded on a etlidiumbromide containing 2% agarose gel and photographed with a UV transilluminator.

For genotyping by Southern blotting, 200 μl of genomic DNA were precipitated by addition of 95% isopropanol, washed once in 70% ethanol, airdried and resuspended in 20 μl of 10 mM Tris-HCl. For the subsequent procedure, see Southern blotting.

Southern Blotting

Genomic DNA was digested by the appropriate restriction enzyme at 37° C. ON separated on a 0.8% agarose gel and subsequently transferred via the alkaline transfer method to a positively charged nylon membrane (Hybond N, Amersham Pharmacia, Little Chalfont, UK). After baking for 1 hour at 80° C., the membrane was prehybridized at the appropriate temperature for 6 hours in hybridization buffer (1M NaCl, 50 mm Tris HCl pH 7.5, 10% dextransulfate, 1% SDS and 100 μg/ml sonicated salmon sperm) and subsequently hybridized ON with a ³²P-dCTP-labeled cDNA probe (using the High Prime DNA Labeling Kit from Boehringer Mannhein/Roche). After several stringency washes with SSC/SDS-solutions the membrane was exposed to a BioMax X-ray film (Kodak, Rochester, N.Y.) at −70° C. and developed to visualize the signal.

Northern Blotting

Total RNA was isolated using the RNeasy Kit (Qiagen) and eluted in nuclease-free water. Equal amounts of total RNA (e.g. 10 μg) were denatured at 65 ° C. for 15 min, separated on a 1% agarose gel with 2.2 M formaldehyde and subsequently transferred via alkaline transfer to a positively charged nylon membrane (Hybond N, Amersham). After baking for 1 hour at 80° C., the membrane was prehybridized at 45° C. for 2 hours in prehybridization solution and subsequently hybridized ON with a 32 P-dCTP-labeled MIF, GAPDH-, β-Actin-cDNA probe (using High Prime DNA Labeling Kit, Boehringer Mannheim/Roche).

After two 5 min washes with 2×SSC/0. 1% SDS at room temperature and two 15 min washes with 0.1×SSC/0.1% SDS at 45° C. the membrane was exposed to a BioMax X-ray film at −70° C. for 2-8 hours and developed to visualize the signal.

Preparation of Murine Embryonic Fibroblasts

Female MIF−/−, MIF+/+, MIFpg/pg or MIFcs/cs mice were housed with corresponding homozygous males and checked for the presence of vaginal plugs before 10 a.m. every morning. The day of finding a vaginal plug was considered day 0.5 of pregnancy. Mothers were sacrificed by CO₂ on embryonic day 14.5 and the embryos were removed from the uterus and the amniotic sack. After excising the blood-forming organs and the head, the embryonic bodies were cut into fine pieces and digested in Trypsin/EDTA and glass beads at 37° C. for 30 min under rotation. After addition of an equal volume of DMEM/10% FCS, cell suspensions were filtered through nylon mesh, counted and cultured in 15 cm dishes at a density of 3×10⁶ cells/dish. Only fibroblasts from passages 2 to 5 were used in experiments.

Retroviral Constructs and Infection of MIFs

Replication-defective retroviral expression vector REBNA was used as described in (240). The following cDNAs were used for retroviral expression: E1A12S, murine c-myc, and human H-ras^{v12}. Retroviral stocks were produced as described (240). For viral infections, 105 MIFs plated on a 6-cm dish were incubated overnight with an appropriate amount of the corresponding retrovirus. Multiple infections were performed sequentially, with a 12 to 24-hr interval between each infection. Typically, the efficiency of infection of primary fibroblasts ranged from 60-70%, while the efficiency of infection of immortalized cells was greater than 95%. Cells were analyzed for the corresponding protein levels two to four days post infection.

Growth Experiments

MEFs were cultured in DMEM/10% FCS in triplicates at a density of 6×10⁵ cells/6-cm dish (=high density) or 5×10⁵ cells/15-cm dish (=low density). Cells were harvested in regular intervals (high density: every 3 days; low density: every 2 days), counted in a Coulter Counter and again seeded at the initial density.

Confluency Experiments

1×10⁶ MIFs were plated in a 10 cm dish in duplicates and cultured 5 days beyond confluency before harvesting and counting.

Thymidine Incorporation

1×10⁵ MEFs were plated in triplicates in 6 well-plates and grown for 24 hours to subconfluency (60%). Serum was removed by two washes with 1×PBS and cells were serum-starved for 48 hours in DMEM/0.1% FCS. Cells were stimulated by culture in 110% FCS and allowed to incorporate ³H-thymidine (final 5 μCi/mL) for one hour at 4, 8, 12, 16, 20 and 24 hours after serum addition. Cells were trypsinized, counted, transferred to 96 well plates, harvested (Packard-Harvester Filtermate 196, UniFilter-96, GF/C, Packard, Meriden, Conn.) and analyzed in a Beckman LS6500 Scintillation Counter (Beckman, Fullerton, Calif.). The counts per min were adjusted to an equal number of cells.

Focus Formation Assay

3×10³ transformed fibroblasts were mixed with 3×10⁵ uninfected MIFs (feeder MIFS) and plated in duplicates onto 6-cm dishes. Cells were maintained in DMEM supplemented with 5% FCS and 1× antibiotic/antimycotic (Gibco). Growth medium was changed every three days. In twelve to fourteen days, transformation efficiency was evaluated by counting of individual colonies. Plates were stained with Giemsa and photographed.

Statistical Analysis

The statistical analysis was performed using student's t-test. The significance of the survival experiments was determined by the X²-test.

Having generally described this invention, a further understanding can be obtained by reference to certain specific examples which are provided herein for purposes of illustration only and are not intended to be limiting unless otherwise specified.

### EXAMPLES

### Example 1

### Generation of the MIF Floxed- and of the MIF Knockout-Mouse

The object was construction of a targeting vector that would ensure 100% loss of function when deleting the entire MIF gene (promoter and all exons) and normal expression of the MIF gene when flanked by loxP sites.

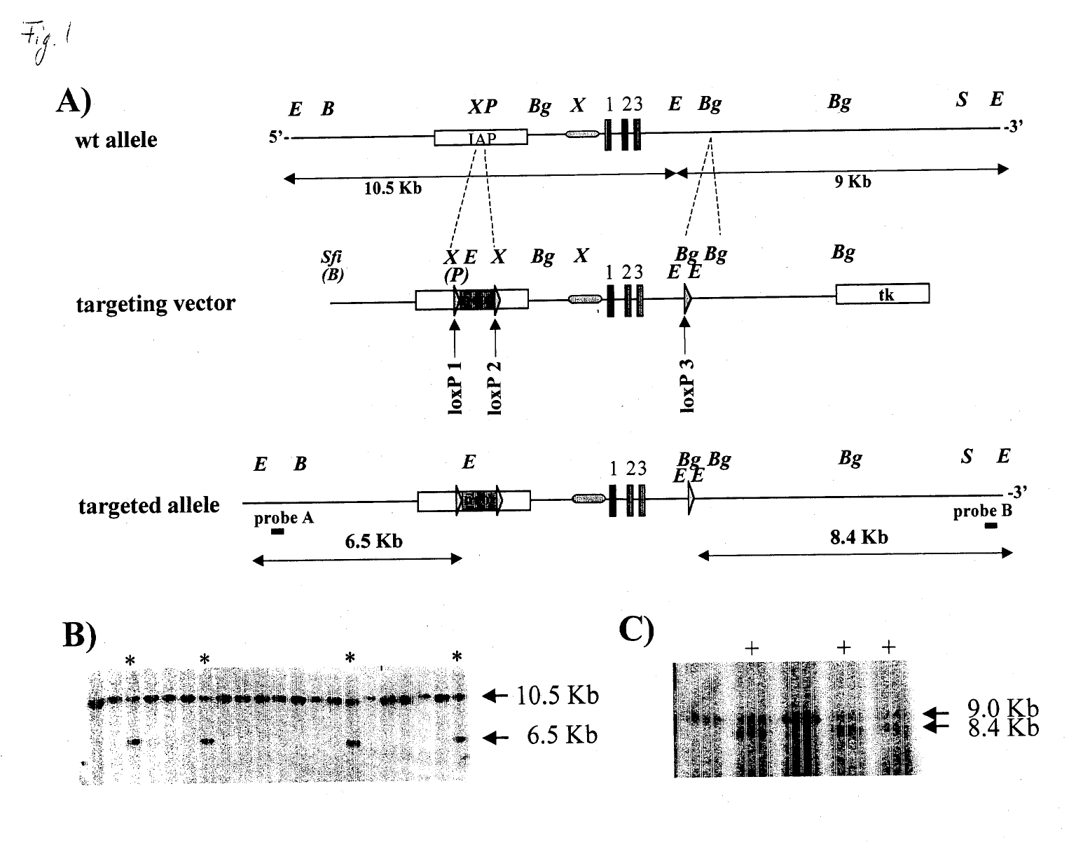

An MIF-containing P1-genomic clone was obtained from the mouse strain 129/Sv. The Neo-cassette for positive selection flanked by loxP sites was placed into an intracisternal A-particle (a type of retrotransposon) which is located upstream of the MIF promoter and has been described to be highly mutated and non-functional. A third loxP site was placed 1.2 Kb downstream of the MIF gene. Accordingly, loxP sites 2 and 3 flank a 5.5 Kb genomic fragment, which contains the MIF-gene. The targeting vector also contained tk for negative selection (FIG. 1).

Embryonic stem cells (ES-cells) were targeted from the strain C57B1/6 (Bruce-4 ES-cells). Targeted cells were selected by culture in G418 for 9 days and enriched for homologous recombinants using gancyclovir from day 5-7.

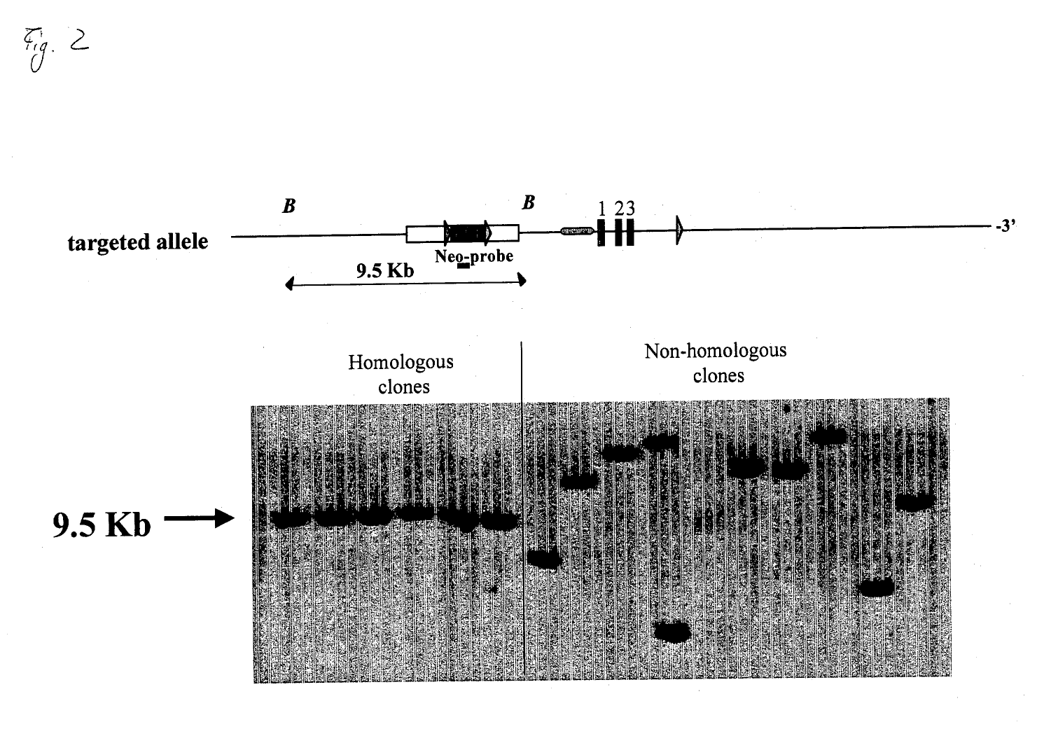

Southern blotting of EcoRI-digested genomic DNA with an external, upstream probe (probe A in FIG. 1A) identified several ES-clones that had an homologous integration of the vector and showed the expected 6.5 Kb fragment in addition to the 10.5 Kb wildtype allele (FIG. 1B). The overall frequency of homologous integration was 15%. The cointegration of the distant third loxP-site and the integrity of the downstream end of the MIF locus were verified by Southern blotting with the external probe B, which showed a double band of 8.4 Kb (targeted) and 9.0 Kb (wildtype) in 61% (24/39) of the homologous clones (FIG. 1C). Additional non-homologous integration of the targeting vector did not occur in the genome of the selected homologous clones as evidenced by BamH 1-digest of the genomic DNA and screening with a neomycin-specific cDNA probe by Southern blotting. All homologous clones showed the expected, single fragment of 9.5 Kb size, whereas the non-homologous clones showed one to several fragments of varying sizes (FIG. 2).

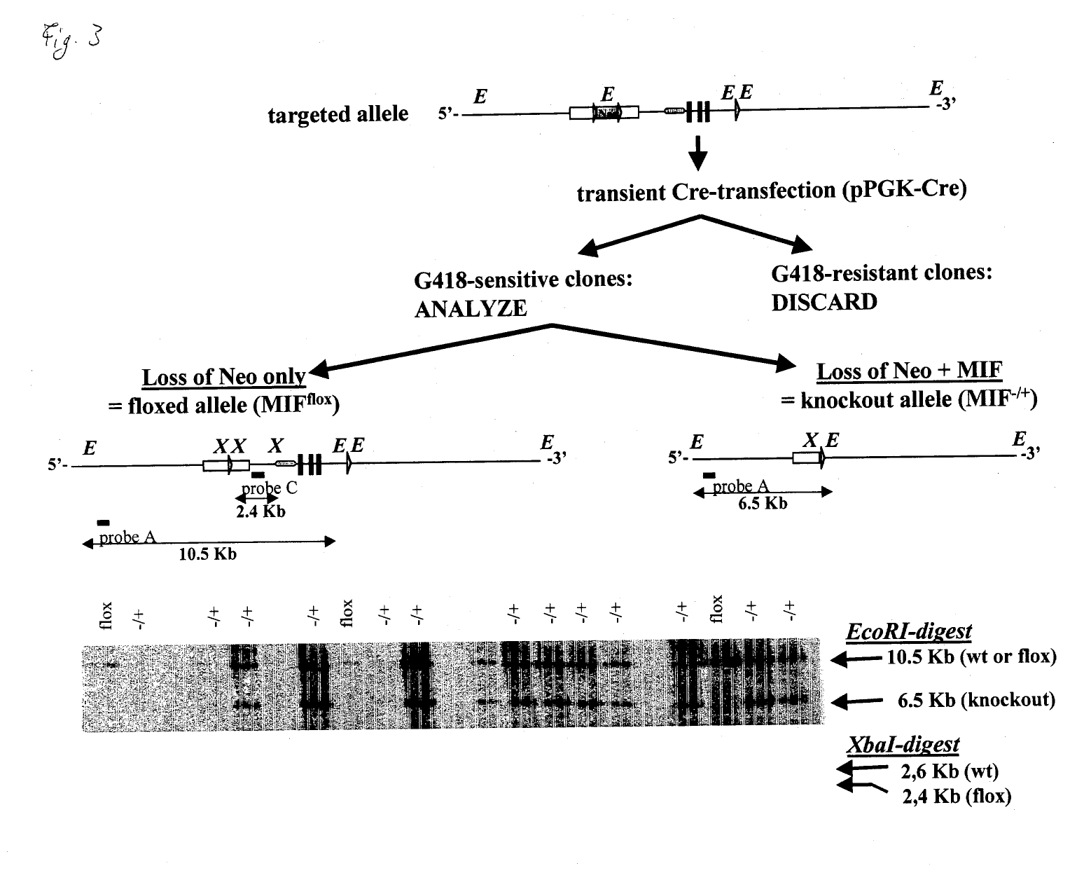

Having successfully identified the ES cell clones with a single homologous recombination event and a cointegrated third loxP site, our next aim was now to remove the neomycin selection cassette, which was no longer necessary, and to obtain ES cell clones, in which MIF was either flanked by loxP sites (“floxed”) or deleted. We transiently transfected two homologous clones with a plasmid expressing Cre under control of the PGK-promoter (pPGK-Cre, kindly provided by K. Rajewsky). ES cell clones that became then G418-sensitive were selected and analyzed by Southern blotting whether Cre had excised the Neo selection cassette alone (=allele with MIF flanked by loxP sites, MIF flox) or the selection cassette and the MIF gene together (=knockout allele, MIF). The knockout allele could be distinguished from the wildtype or the floxed allele by demonstrating the presence of the 6.5 Kb fragment after hybridization of EcoRI-digested genomic DNA with probe A. The distinction between the floxed and the wildtype allele was based on the presence of a 2.4 Kb fragment in XbaI-digested DNA detected with the internal probe C. This procedure yielded 3 ES cell clones, which carried a floxed MIF allele, and 13 clones, which carried a knockout allele (FIG. 3).

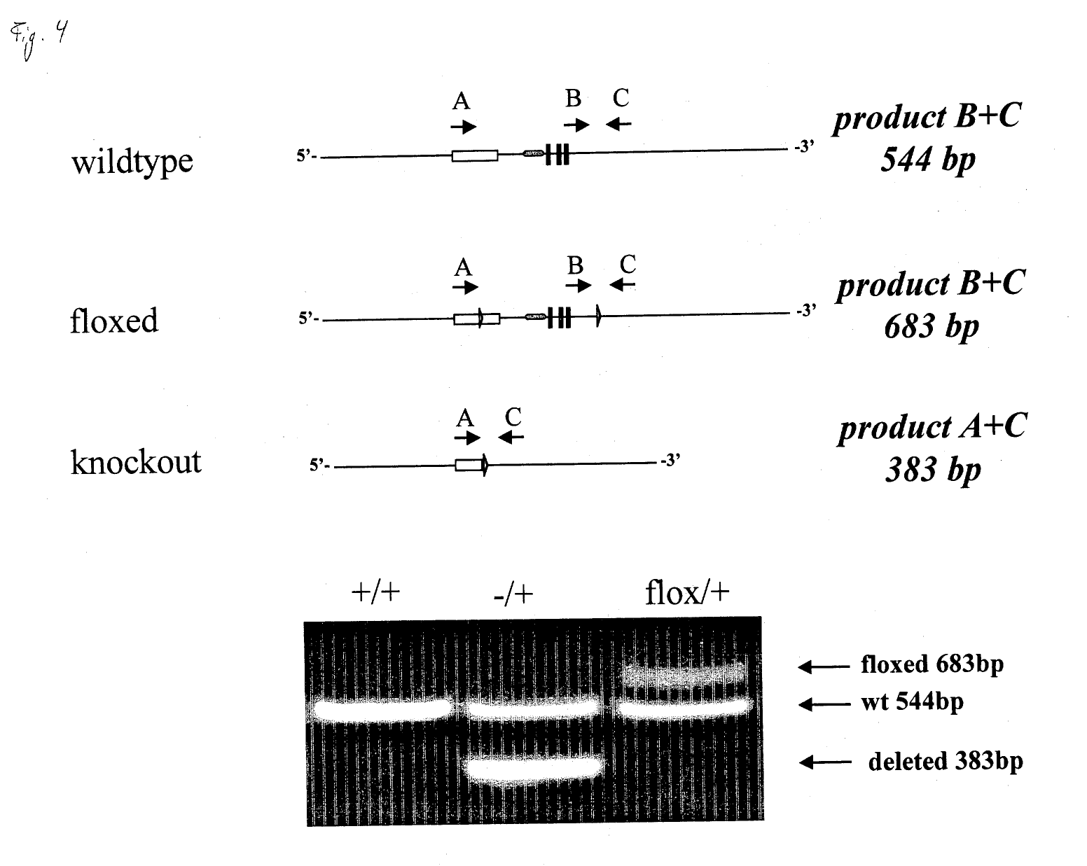

The floxed and the knockout clones were injected into BALB/c blastocysts and obtained several chimeric mice for each clone. Male chimeras were bred to C57B1/6 females and pups that had inherited the targeted allele were identified based on their coat color (black=germline transmission, agouti=no germline transmission) and genotyping by PCR (FIG. 4). Heterozygote pups were bred with each other to homozygosity. Due to the use of C57B1/6 ES cells, these mice are genetically pure C57B1/6.

Validation of the Targeting Success

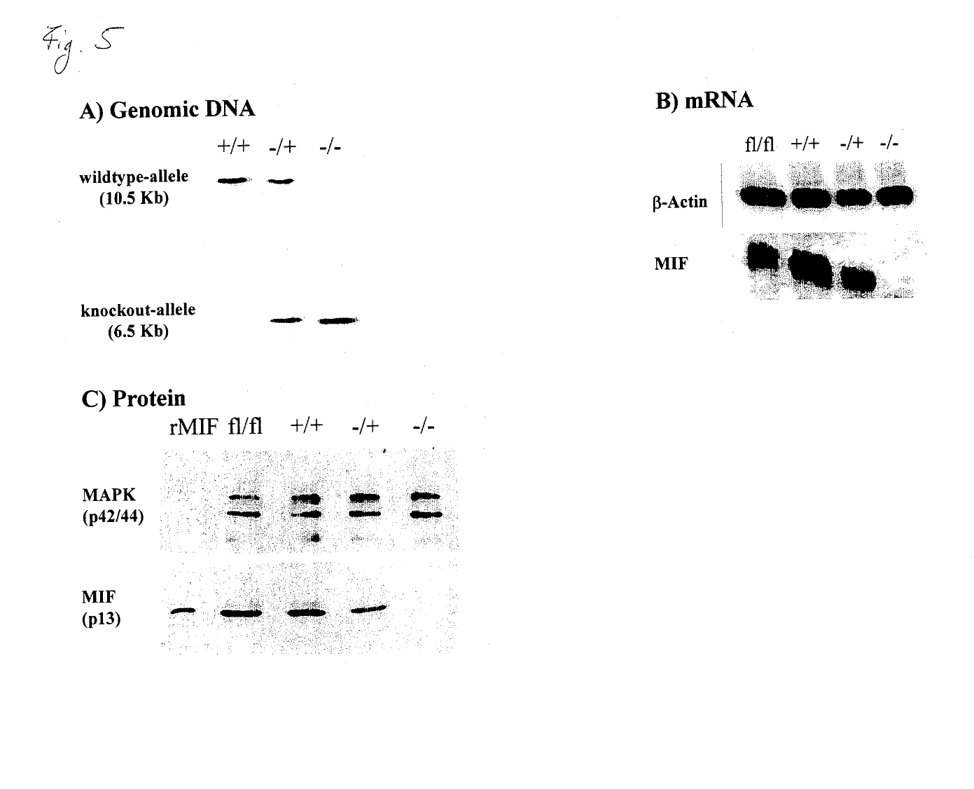

The effect of the targeting was evaluated on the levels of genomic DNA, mRNA and protein in livers of LPS-challenged MIF^{+/+}, MIF^{-/+} and MIF^{-/-} mice (FIG. 5). Sixteen hours after an i.p. injection of a sublethal dose of E. coli LPS (15 mg/kg), mice were sacrificed and the livers were processed to obtain genomic DNA, mRNA and protein. Southern blotting with probe A in EcoRI-digested genomic DNA confirmed the presence of a single 6.5 Kb band in the MIF^{-/-} mouse (FIG. 5A). Northern analysis with a full length MIF cDNA probe (exons 1-3) as well as Western blotting with the polyclonal anti-MIF antibody R102 demonstrated the complete absence of MIF mRNA and protein in these animals (FIGS. 5B-C).

### EXAMPLE 2

### Generation of the MIF p1g- and of the MIFc60s-Mouse

Mutational Strategy

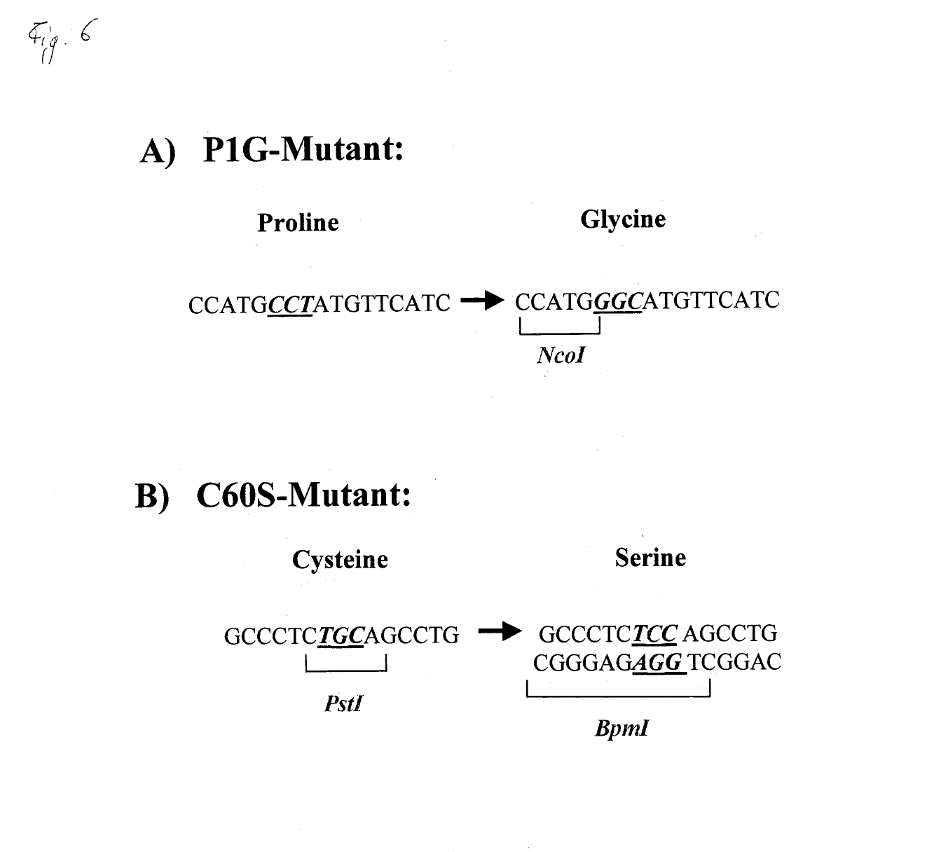

The mutation of proline to the smallest amino acid glycine has been shown by X ray crystallography to preserve the structure of the pocket while eliminating the isomerase activity of MIF. The N-terminal proline is encoded by the codon CCT in the mouse. According to codon usage in the mouse the codon GGC was a frequently used codon for glycine. Replacing the triplett CCT by GGC also created a novel restriction site for the enzyme NcoI (FIG. 6A).

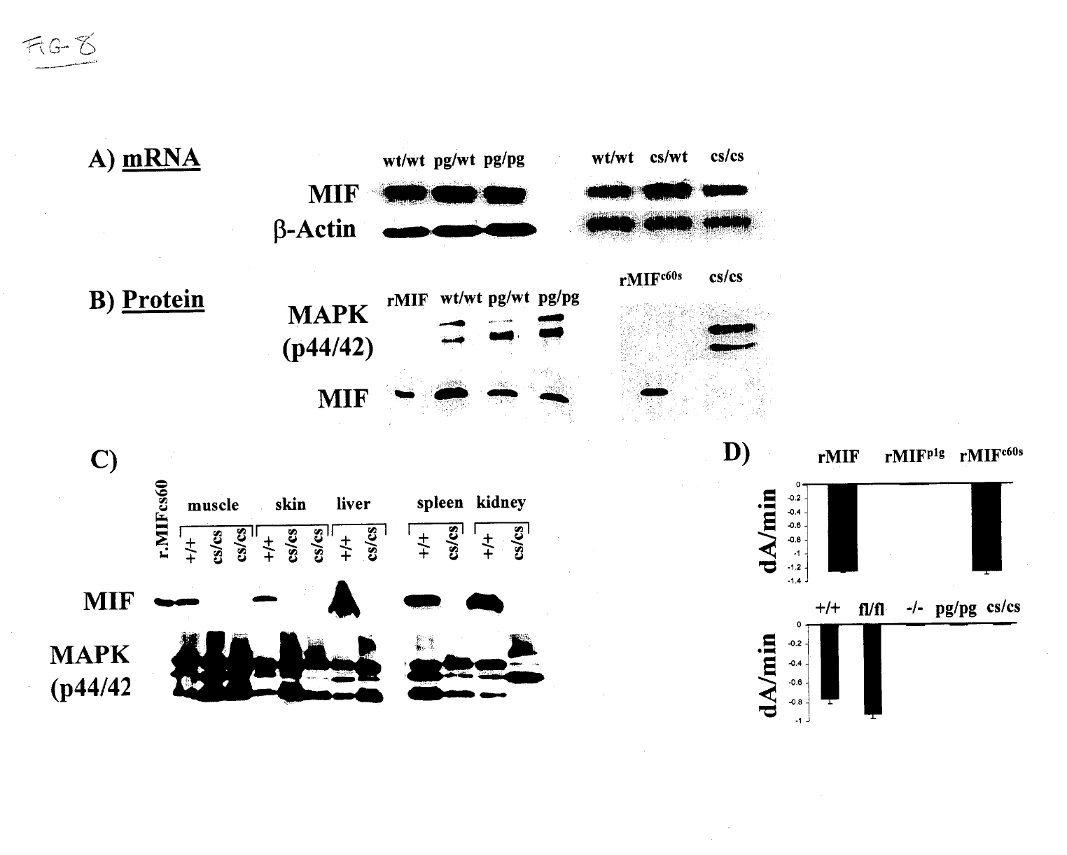

The efficiency of the P->G mutation in destroying the isomerase activity was verified first in recombinant mutant proteins in the tautomerase assay with dopachrome methylester as substrate. The P->G protein had no detectable activity (FIG. 8D). Cysteine 60 was mutated by a single point mutation (G->C) to serine, another polar amino acid. This mutation destroyed the original PstI restriction site and created a new restriction site for BpmI on the opposite strand (FIG. 6B).

Mutagenesis of the MIF by Gene Targeting

A Cre-containing selection cassette was chosen for the targeting vector, which would delete itself after identification of the correct ES-cell clones, leaving only a single loxP-site in the genome. Such a cassette (pACN) contains the selectable marker Neo driven by the polymerase II promoter as well as the Cre recombinase under control of the testis-specific tACE promoter (tACE=testis-specific angiotensin converting enzyme). The two genes are flanked by loxP sites, which subsequently allow the self-excision of the cassette by Cre when the ES-cell undergoes differentiation to sperm cells. Thus, offspring with germline transmission of the targeted allele from the chimeric mice will inherit the mutation and a loxP site.

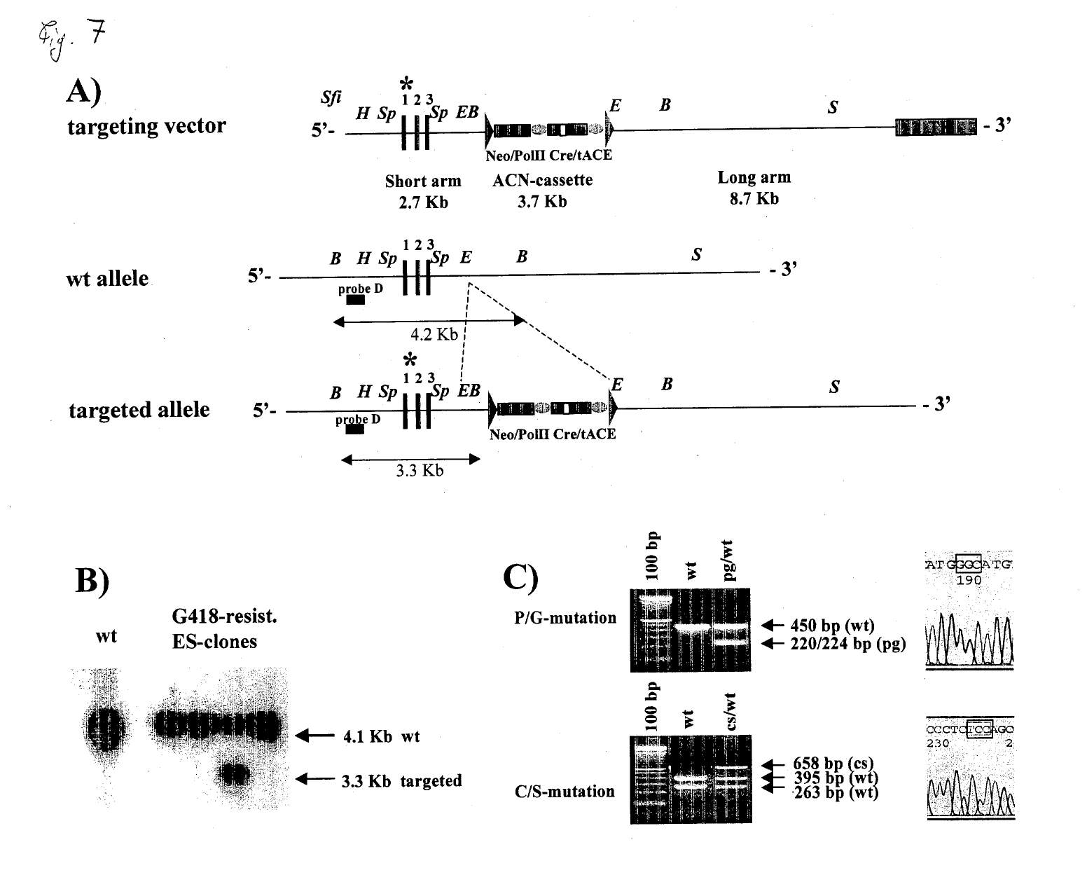

Each of the mutations were engineered separately in a 2 Kb MIF-containing SpeI-DNA fragment by PCR mutagenesis and sequenced the entire insert to make certain that the desired mutation was present and that the PCR had not introduced any additional random mutation. The ACN-cassette was placed downstream of the MIF-polyA and thymidine kinase was included at the 3′-end of the targeting vector (FIG. 7A). As in the MIF knockout, we transfected C57B1/6 ES-cells (Bruce-4) and obtained several homologous recombinants for each construct, which showed the expected additional 3.3 Kb band in BamH 1-digested genomic DNA hybridized with the external probe D (FIG. 7B). The presence of the mutation in the homologous ES-cells was confirmed in two ways: by PCR and subsequent restriction digest as outlined in FIG. 5 as well as by direct sequencing of the mutation-containing PCR fragments (FIG. 7C).

The presence of additional vector integrants were excluded by HindIII-digest and hybridization with a neo-probe and then injected one ES-clone for each mutation into BALB/c blastocysts. Several male chimeras transmitted the mutant allele to their offspring and these heterozygous mice were bred to homozygosity.

Expression of Mutant MIF

In order to ensure that potential phenotypes in the mutant mice were not due to insufficient expression of mutant MIf, MIF expression levels were tested in male wildtype, heterozygous and homozygous animals by Northern and Western blotting. MIF^{pg} mRNA was expressed at levels equivalent to wildtype MIF, the MIF^{pg} protein was slightly reduced (FIGS. 8A+B). However, whereas MIFCS mRNA was expressed normally and the polyclonal anti-MIF antibodies detected recombinant MIF^{cs60} there was no detectable MIF^{cs} protein in homozygous mice even in a variety of tissues such as muscle, skin, liver, spleen and kidney (FIG. 8C).

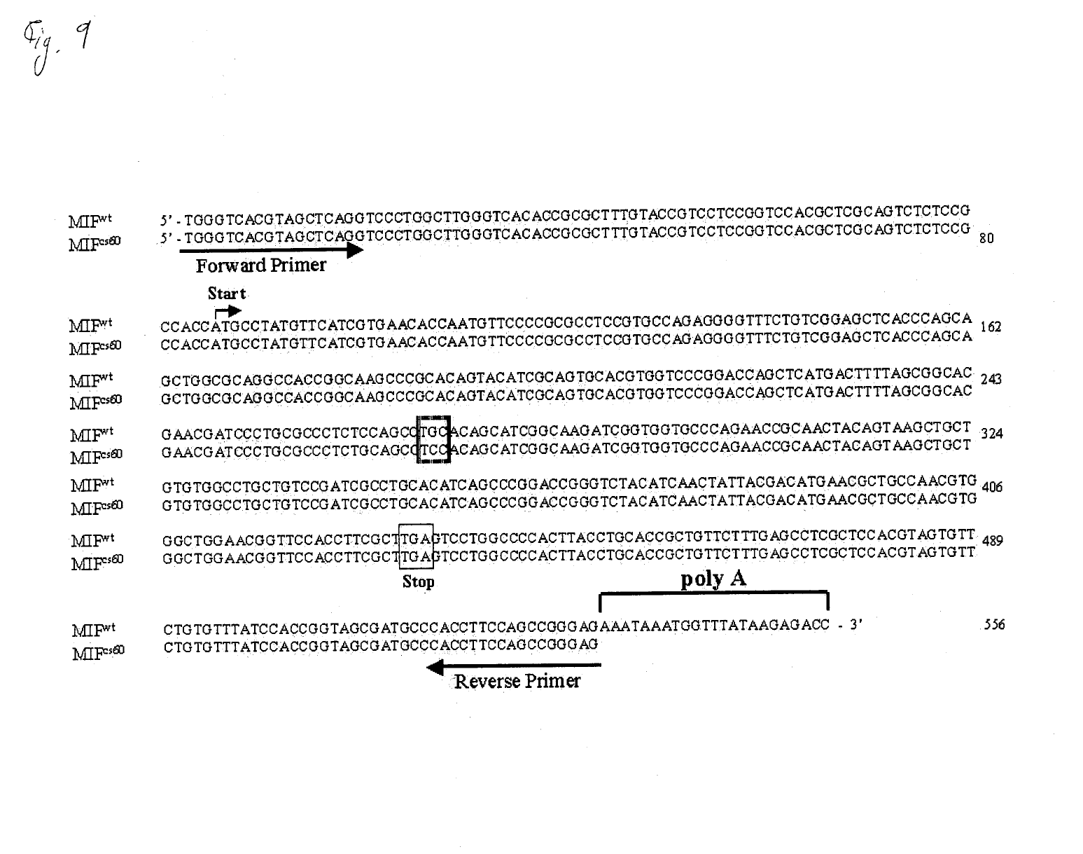

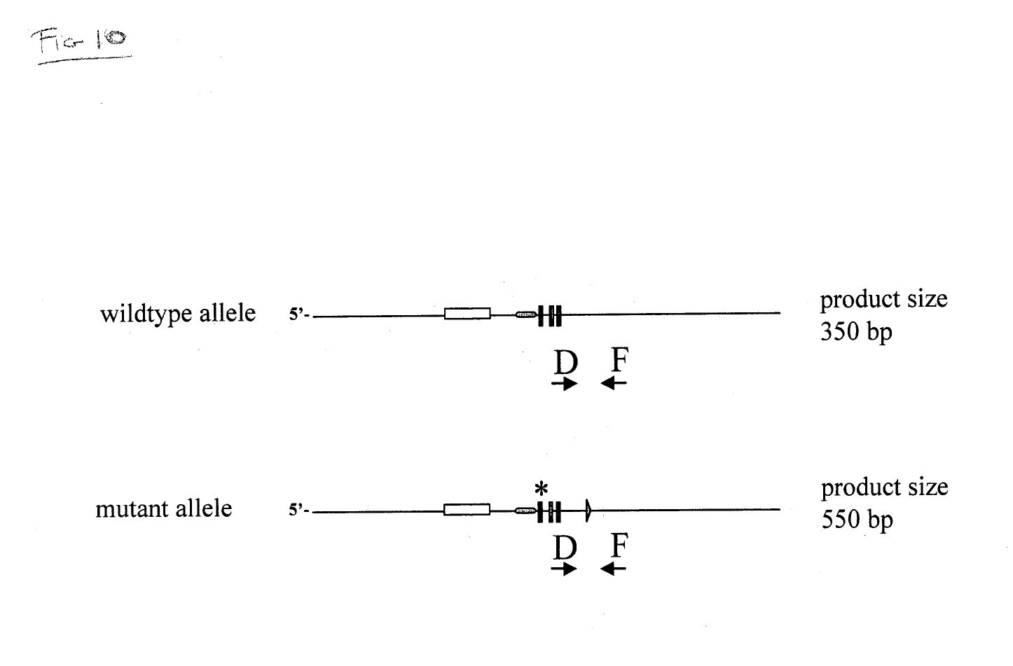

In order to exclude the possibility that an additional mutation in the MIF^{cs60} gene might be causing a premature stop of translation or a frameshift, the MIF^{cs60} mRNA were cloned from the beginning of the 5′-untranslated region to the beginning of the polyA tail from spleen of a MIFcs/cs mouse. Sequencing showed that there was no mutation, which could account for the loss of protein expression besides the cs6O-mutation (FIG. 9).

MIF Gene Deletion Does not Lead to Endotoxin Resistance in C57B1/6

In contrast to previous (21) and these experimental results that were based on the use of recombinant protein and monoclonal antibodies, the MIF^{-/-} macrophages displayed a normal cytokine response in response to stimulation with LPS in vitro. Additionally, the MIF^{-/-} mice were not resistant to the lethal effects of endotoxin in vivo. These results are in agreement with the conclusion of Honma et al. who also did not note an effect of MIF deficiency on LPS-elicited cytokine production or lethality with genetically engineered mice on a mixed 129/C57 background (237). However, their and these results are in contradiction to Bozza et al. who were the first to report that deletion of the MIF gene in a mixed 129/C57 background conferred significant protection from LPS shock and was associated with reduced levels of the crucial LPS mediator TNF-α (236).

| | Bozza et al. | Honma el al. | Fingerle et al. |
|---|---|---|---|
| Source of genomic | 129/SvJ | 129/SvJ | 129/SvJ |
| DNA | | | |
| ES-cells | J1 | R1 | Bruce4 |
| | (129/SvJae) | (129/Svx | (C57B1/6) |
| | | 129/Sv-CP)F1 | |
| Targeting strategy | Neo- | Neo-replacement | Cre-IoxP |
| | replacement | | |
| Deletion in the MIF | 3′-region of | 3′-region of | promoter + all |
| locus | exon-2 | exon-1 | exons |
| | -> 3′-UTR | ->5′-region of | |
| | | intron-1 | |
| Resulting genetic | mixed | mixed | pure |
| background | 129/SvJae/C57 | 129 | C57B1/6 |
| | B16 | Svx129SvJ/C57 | |
| | | B16 | |
| Presence of neo- | yes | yes | yes |
| cassette | | | |

Differences Between MIF Knockout Mice

The knockout mice created by Bozza et al. (236), Honma et al. (237) and of the instant invention differ in various aspects such as the ES-cells used, the targeting strategy, the size of the deletion in the MIF locus, the resulting genetic background as well as the presence or absence of the neo-selection cassette. The information about the genetic background of the ES-cells is taken from Simpson et al. Nature Genetics, 16:19-27, 1997.

The proof of MIF gene deletion in the instant invention was confirmed by demonstrating the loss of the MIF locus in the genomic DNA and by the absence of MIF MRNA and protein. The MIF^{-/-} mice do not produce any detectable amount of MIF. In several fibroblast based bioassays such as cell proliferation, confluency or malignant transformation, the inventive MIF^{-/-} MEFs display the same phenotype as the MIF^{-/-} MEFs created by Bozza et al., suggesting that indeed both knockout mice are valid models of MIF deficiency.

The cause of the discrepancy in the LPS responsiveness studies is not immediately obvious, but several possibilities exist that might explain this difference. The integration of a gene targeting vector usually occurs as a single integration event, but occasionally the vector integrates in an additional non-homologous fashion at a different site in the genome. Bozza et al. did not perform an analysis for such additional integrants in the ES cell clone that give rise to that knockout mouse. Therefore, the possibility that their gene-targeting vector could have disrupted an additional, LPS-related gene cannot be dismissed.

There are other differences between the three gene targeting strategies that could account for the contradictory outcome in endotoxemia such as the ES-cells used, the presence or absence of the selectable marker, the size of the DNA deletion in the MIF-locus or the genetic background of the resulting mice. As it is known that the selectable marker of replacement type vectors may disturb the regulation and splicing of genes adjacent to the targeted gene (184;185), the neomycin cassette was excised through Cre-loxP mediated recombination in vitro in the ES-cells. By contrast, the targeting strategy of Bozza et al. requires the continued presence of the neomycin cassette. In close vicinity to the MIF gene lie the genes glutathione S-transferase-1 and -2 (GSST-1 and -2), which participate in balancing the redox status (321). The importance of the redox system in counteracting the detrimental effects of reactive oxygen and nitrogen species during endotoxemia and sepsis suggests that these genes could be involved in the pathogenesis of septic shock (322).

The MIF^{-/-} mice according to the invention were created on a pure C57B1/6 background whereas the other MIF^{-/-} strains were analyzed in a mixed 129/C57 background. It is well known that genetic background is a major determinant of LPS sensitivity. C57B1/6 mice are known to tolerate higher doses of endotoxin than BALB/c or 129/Sv mice (323). The genes or the gene regulatory elements that are responsible for this different sensitivity are not yet known. Furthermore, it has been shown recently that 129 substrains and ES-cells derived from them exhibit great genetic variability which has a negative impact on the availability of the appropriate controls and the preparation of inbred animals (324). It is therefore possible that the action of MIF in LPS-induced shock depends on a genetic background and that it is not present or less pronounced in the C57B1/6 strain. Preliminary results in the F6 generation of the MIF^{-/-} BALB/c mice, in which thωe antibody studies showed the protective effect of anti-MIF, however show that these mice are equally sensitive to LPS compared to wild-type littermates. This suggests that mechanisms that are independent from genetics might be responsible for the different outcomes.

The conclusions drawn from experiments with MIF-deficient mice according to the invention differ from previous experiments, which used anti-MIF antibodies in BALB/c mice (15). Three scenarios may account for this observation. First, genetic MIF deficiency is a life-long event whereas anti-MIF therapy constitutes an acute intervention. If MIF is part of a redundant system, other molecules may compensate for it. In LPS shock, not only TNF-α, but also IL-1, IL-6, IL-8, IFN-γ, prostaglandins, leukotrienes as well as reactive oxygen and nitrogen species participate in the host response to LPS (334;335). These mediators act alone or in combination to activate the deleterious effects of LPS. Second, anti-MIF may directly or indirectly cross-react with the function of another molecule involved in pathogenesis of LPS shock. Third, therapy with anti-MIF antibodies may lead to the formation of immune complexes, which in turn may modulate the host LPS-response. Immune complexes are potent inducers of IL-1 receptor antagonist (IL-IRA) (336), which acts to dampen the inflammatory response (337).

Thus, the MIF^{-/-} mice according to the invention presents an improved model for conducting studies with MIF deficient mice as the MIF deficient mice according to the invention are not hampered by the same drawbacks associated with ordinary mice treated with anti-MIF antibodies to remove or reduce MIF activity in vivo.

MIF as Tautomerase

The animal models according to the invention also address the question whether the tautomerase activity of MIF underlies its biological activity, as has been suggested (138). Although the structural similarity of MIF with bacterial enzymes and the evolutionary conservation of the “catalytic pocket” provided some grounds for this hypothesis, several questions remained unanswered. Firstly, a physiological substrate has not been identified. Secondly, experiments with recombinant MIF mutants deficient in tautomerase activity designed to test the bioactivity of these proteins have been hampered by the difficulty that there is no easy and robust bioassay for MIF activity. (138) demonstrates that MIFP^{pg} lacks bioactivity in a neutrophil-based bioassay, however the validity of this assay for MIF bioactivity remains questionable. The majority of the results obtained in experiments such as glucocorticoid overriding or inhibition of macrophage migration mitigate against tautomerase activity being required for the biological activities of MIF (139; 144).

Testing of the tautomerase-hypothesis in vivo is made possible using animal models of the invention made by the knock-in approach. A mutation (proline 1 to glycine) was engineered in C57B1/6 mice which was shown to minimally disturb the MIF structure while eliminating the tautomerase activity (141). As predicted this mutation completely abrogates the tautomerase activity in mouse liver extracts despite a fairly normal expression of the mutant protein in vivo. MIFPG MEFs was then tested in comparison with MIF-1- and wildtype MEFs and it was found that the combination of E1A/H-ras was able to transform MIFPG fibroblasts as efficiently as wildtype cells. Thus, it can be concluded that MIF's tautomerase activity is not likely to account for its biological activity. Alternatively, the tautomerase activity of MIF may not be required for ras-mediated transformation.

To date, most screening efforts to identify inhibitors of MIF rely—at least initially—on the tautomerase activity as the “catalytic pocket” is nevertheless likely to represent the biologically crucial area of MIF. Another approach than specifically targeting the function of proline 1 may be to develop compounds which obstruct the entire pocket.

A Role for Cysteine 60 in MIF Folding

MIF contains a conserved CXXC-motif in position 57-60 (Cys-Ala-Leu-Cys) as well as a single cysteine in position 81. CXXC-sequence motifs have been shown to be the catalytic center of thiol-protein oxidoreductases such as thioredoxin (145;149;338), protein disulfide isomerase (339), glutaredoxin (340) or DsbA (341) and are based upon the formation or reduction of a disulfide bridge between the adjacent cysteines.

The main chemical reaction involved in disulfide-bond studies is thiol/disulfide exchange (R₁S-+R₂SSR₃→R₂S⁻+R₁SSR₃), in which the thiolate anion displaces one sulfur of the disulfide bond. Thiol/disulfide exchange reactions can occur intramolecularly as well as intermolecularly (44).

In analogy to the knock-in mutation of proline 1, the mutation of cysteine 60 to serine (MIF^{c60s}) is also engineered by a similar knock-in approach. The selection cassette was removed during spermatogenesis by Cre-loxP-mediated recombination (243). The expression of this mutant protein was reduced to undetectable levels by Western analysis while mRNA expression appeared normal. Several possibilities may account for such a phenomenon: First, protein translation may be impaired; second, proper folding might require Cys60 and third, protein degradation may be increased.

Immediately after translation, proteins appear to fold rapidly into a structure in which most of the final secondary structure (α-helices and β-sheets) has formed and in which these elements of structure are aligned in roughly the right way. This usually open and flexible conformation, which is called a molten globule, is the starting point for a relatively slow process in which many side-chain adjustments occur in order to form the correct tertiary structure. Molecular chaperones (e.g. heat-shock proteins 60 and 70, hsp60, hsp70) are special proteins in cells whose function is to help other proteins fold and assemble into stable, active structures. Damaged or misfolded proteins are recognized and degraded by ubiquitin-dependent proteolytic systems (342).

Cysteines are important structural components for protein folding. Disulfide bond formation is a preferred mechanism of folding and its importance has been demonstrated in a great variety of proteins (e.g. bovine pancreatic trypsin inhibitor BPTI (343), ribonuclease A (344), (α-Lactalbumin (345)).

The existence of a disulfide bond between Cys57 and Cys60 is still somewhat controversial. There is some biochemical evidence for the existence of this cystine bond (47;150), X ray crystallographic results show that these two cysteines are too far apart to react with each other (38). Cysteine 57 is buried in the hydophobic core of the protein and is unlikely to react with the environment. Cysteine 60 faces towards the surface of MIF and could potentially react with another molecule. Cysteinylation of Cys-60 as a posttranslational modification of MIF which leads to a conformational change and rendering MIF bioactive has also been shown (48). In the case of a posttranslational mechanism, it is useful to determine how the mutation of cysteines influences the folding process of MIF and what might be the thiol-reactive binding partner. For therapeutic purposes, this finding may provide the basis for a different class of MIF small drug inhibitors, which interfere with folding of MIF instead of blocking the conserved pocket.

In developing the present invention, studies were conducted to identify macrophage migration inhibitory factor as a cell cycle regulatory protein. Based on studies in MIF^{-/-} embryonic fibroblasts from the knockout animal model of the invention, it is shown that MIF deficiency influences the growth characteristics and the proliferative capacity of MEFs in a strain-dependent fashion. Importantly, the lack of MIF greatly impairs the Ras-mediated oncogenic transformation of fibroblasts.

Furthermore, in vivo experiments in the rat (not shown) demonstrate that MIF expression in the adrenal is positively regulated by glucocorticoids. Therapeutic doses of glucocorticoids lead to tissue-and time-specific changes in MIF expression that can be associated with the adaptation of sensitive organs to the anti-mitogenic influence of glucocorticoids.

These results implicate MIF as contributing factor in development, growth, differentiation and tumorigenesis. The in vivo-mutational analysis of MIF structure and function gives evidence that MIF does not function as tautomerase. MIF folding appears to be crucially dependent on the presence of cysteine 60. These structure-function relationships provide important information for the possible mechanism of action of MIF as well as for the future development of MIF inhibitors.

The forgoing analyses relate to mice in which the gene encoding macrophage migration inhibitory factor (MIF) has been deleted, nullified or mutated. The particular mouse strains that are described include:

1. C57B1/6J-TgH(MIFflox)1Grf mouse (“MIFflox-mouse”) which can be used to generate inducible and tissue-specific MIF knockout mice.

2. C57B1/6J-TgH(MIFdel)2Grf mouse (“MIF knockout mouse”) which can be used to investigate and to analyze the effect of MIF deficiency in vivo.

3. C57B1/6J-TgH(MIFpg)3Grf mouse (“MIF plg-mouse”) which can be used to test the biological role of proline 1 of MIF.

4. C57B1/6J-TgH(MIFcs)4Grf mouse (“MIF c60s-mouse”) which can be used to test the biological role of cysteine 60 of MIF.

While this invention has been particularly shown and described with references to preferred embodiments thereof, it will be understood by those skilled in the art that various changes in form and details may be made therein without departing from the scope of the invention encompassed by the appended claims.

### LISTING OF REFERENCES CITED AND SUPPORTING

The following documents are parenthetically cited by number in the specification above.

1. Medzhitov R, Janeway C, Jr. Innate immunity. N Engl J Med 2000; 343:338-44.

2. Medzhitov R, Janeway C A. Innate immunity: impact on the adaptive immune response. Curr.Opin.immunol. 1997;9:4-9.

3. David, J. Delayed hypersensitivity in vitro: Its mediation by cell-free substances formed by lymphoid cell-antigen interaction. Proc Natl Acad Sci USA 56, 72-77. 1966.

4. Bloom, B. R. and Bennett, B. Mechanism of a reaction in vitro associated with delayed-type hypersensitivity. Science 111, 514-521. 1966.

5. Nathan C F. Remold H G, David J R. Characterization of a lymphocyte factor which alters macrophage functions. J Exp Med 1973; 137:275-90.

6. Nathan C F, Karnovsky M L, David J R. Alterations of macrophage functions by mediators from lymphocytes. J Exp Med 1971; 133:1356-76.

7. Churchill W H, Jr., Piessens W F, Sulis C A, David J R. Macrophages activated as suspension cultures with lymphocyte mediators devoid of antigen become cytotoxic for tumor cells. J Immunol 1975; 115:781-6.

8. Thurman G B, Braude I A, Gray P W, Oldham R K, Stevenson H C. MIF-like activity of natural and recombinant human interferon-gamma and their neutralization by monoclonal antibody. J Immunol 1985; 134:305-9.

9. McInnes A, Rennick D M. Interleukin 4 induces cultured monocytes/macrophages to form giant multinucleated cells. J Exp Med 1988; 167:598-611.

10. Cunha F Q, Weiser W Y, David J R, Moss D W, Moncada S, Liew F Y. Recombinant migration inhibitory factor induces nitric oxide synthase in murine macrophages. J Immunol. 1993; 150:1908-12.

11. Weiser W Y, Pozzi L M, Titus R G, David J R. Recombinant human migration inhibitory factor has adjuvant activity. Proc Natl Acad Sci USA 1992;89:8049-52.

12. Suzuki H, Kanagawa H, Nishihira J. Evidence for the presence of macrophage migration inhibitory factor in murine reproductive organs and early embryos. Immunol.Lett. 1996;51:141-7.

13. Kobayashi S, Satomura K, Levsky J M, Sreenath T, Wistow G J, Semba I et al. Expression pattern of macrophage migration inhibitory factor during embryogenesis. Mech.Dev 1999;84: 153-6.

14. Bacher M, Meinhardt A, Lan H Y, Mu W, Metz C N, Chesney J A et al. Migration inhibitory factor expression in experimentally induced endotoxemia. Am.J Pathol. 1997; 150:235-46.

15. Calandra T, Bemhagen J, Mitchell R A, Bucala R. The macrophage is an important and previously unrecognized source of macrophage migration inhibitory factor. J Exp Med 1994; 179:1895-902.

16. Bacher M, Metz C N, Calandra T, Mayer K, Chesney J, Lohoff M et al. An essential regulatory role for macrophage migration inhibitory factor in T-cell activation. Proc Natl Acad Sci USA 1996;93:7849-54.

17. Takahashi A, Iwabuchi K, Suzuki M, Ogasawara K, Nishihira J, Onoe K. Antisense macrophage migration inhibitory factor (MIF) prevents anti-IgM mediated growth arrest and apoptosis of a murine B cell line by regulating cell cycle progression. Microbiol Immunol. 1999;43:61-7.

18. Apte R S, Sinha D, Mayhew E, Wistow G J, Niederkom J Y. Cutting edge: role of macrophage migration inhibitory factor in inhibiting NK cell activity and preserving immune privilege. J. Immunol. 1998;160:5693-6.

19. Chen H, Centola M, Altschul S F, Metzger H. Characterization of gene expression in resting and activated mast cells. J Exp Med 1998; 188:1657-68.

20. Rossi A G, Haslett C, Hirani N, Greening A P, Rahman I, Metz C N et al. Human circulating eosinophils secrete macrophage migration inhibitory factor (MIF). Potential role in asthma. J Clin Invest 1998;101:2869-74.

21. Bernhagen J, Calandra T, Mitchell R A, Martin S B, Tracey K J, Voelter W et al. MIF is a pituitary-derived cytokine that potentiates lethal endotoxaemia. Nature 1993;365:756-9.

22. Waeber G, Calandra T, Roduit R, Haefliger J A, Bonny C, Thompson N et al. Insulin secretion is regulated by the glucose-dependent production of islet beta cell macrophage migration inhibitory factor. Proc Natl Acad Sci USA 1997;94:4782-7.

23. Kudo T. [Identification of macrophage migration inhibitory factor (MIF) in rat gastrointestinal tract and its role in rat stomach]. Hokkaido Igaku Zasshi 1998;73:317-25.

24. Abe R, Shimizu T, Ohkawara A, Nishihira J. Enhancement of macrophage migration inhibitory factor (MIF) expression in injured epidermis and cultured fibroblasts. Biochim.Biophys.Acta 2000; 1500:1-9.

25. Bacher M, Meinhardt A, Lan H Y, Dhabhar F S, Mu W, Metz C N et al. MIF expression in the rat brain: implications for neuronal function. Mol Med 1998;4:217-30.

26. Mizue Y, Nishihira J. Miyazaki T, Fujiwara S, Chida M, Nakamura K et al. Quantitation of macrophage migration inhibitory factor (MIF) using the one-step sandwich enzyme immunosorbent assay: elevated serum MIF concentrations in patients with autoimmune diseases and identification of MIF in erythrocytes. Int.J Mol Med 2000;5:397-403.

27. Mitchell R, Bacher M, Bernhagen J, Pushkarskaya T, Seldin M F, Bucala R. Cloning and characterization of the gene for mouse macrophage migration inhibitory factor (MIF). J Immunol. 1995; 154:3863-70.

28. Bozza M, Kolakowski L F, Jenkins N A, Gilbert D J, Copeland N G, David J R et al. Structural characterization and chromosomal location of the mouse macrophage migration inhibitory factor gene and pseudogenes. Genomics 1995;27:412-9.

29. Budarf M, McDonald T, Sellinger B, Kozak C, Graham C, Wistow G. Localization of the human gene for macrophage migration inhibitory factor (MIF) to chromosome 22q 11.2. Genomics 1997;39:235-6.

30. Kozak C A, Adamson M C, Buckler C E, Segovia L, Paralkar V, Wistow G. Genomic cloning of mouse MIF (macrophage inhibitory factor) and genetic mapping of the human and mouse expressed gene and nine mouse pseudogenes. Genomics 1995;27:405-11.

31. Feo S, Oliva D, Arico B, Barba G, Cali L, Giallongo A. The human genome contains a single processed pseudogene for alpha enolase located on chromosome 1 . DNA Seq. 1990; 1:79-83.

32. Takahashi Y. [Gene expression in the cells of the central nervous system]. Seikagaku 1991;63:253-81.

33. Kobayashi S, Yoshida K, Ohshima T, Esumi N, Paralkar V M, Wistow G J et al. DNA sequence motifs are associated with aberrant homologous recombination in the mouse macrophage migration inhibitory factor (Mif) locus. Gene 1998;215:85-92.

34. Keshet E, Schiff R, Itin A. Mouse retrotransposons: a cellular reservoir of long terminal repeat (LTR) elements with diverse transcriptional specificities. Adv Cancer Res 1991;56:215-51.

35. Paralkar V, Wistow G. Cloning the human gene for macrophage migration inhibitory factor (MIF). Genomics 1994;19:48-51.

36. Metz C N, Bucala R. Role of macrophage migration inhibitory factor in the regulation of the immune response. Adv.Immunol. 1997;66:197-223.

37. Bernhagen J, Mitchell R A, Calandra T, Voelter W, Cerami A, Bucala R. Purification, bioactivity, and secondary structure analysis of mouse and human macrophage migration inhibitory factor (MIF). Biochemistry 1994;33:14144-55.

38. Sun H W, Bernhagen J, Bucala R, Lolis E. Crystal structure at 2.6-A resolution of human macrophage migration inhibitory factor. Proc Natl Acad Sci USA 1996;93:5191-6.

39. Kato Y, Muto T, Tomura T, Tsumura H, Watarai H, Mikayama T et al. The crystal structure of human glycosylation-inhibiting factor is a trimeric barrel with three 6-stranded beta-sheets. Proc Natl Acad Sci USA 1996;93:3007-10.

40. Sugimoto H, Suzuki M, Nakagawa A, Tanaka I, Nishihira J. Crystal structure of macrophage migration inhibitory factor from human lymphocyte at 2.1 A resolution. FEBS Lett. 1996;389: 145-8.

41. Muhlhahn P, Bemhagen J, Czisch M, Georgescu J, Renner C, Ross A et al. NMR characterization of structure, backbone dynamics, and glutathione binding of the human macrophage migration inhibitory factor (MIF). Protein Sci 1996;5:2095-103.

42. Magi B, Bini L, Liberatori S, Marzocchi B, Raggiaschi R, Arcuri F et al. Charge heterogeneity of macrophage migration inhibitory factor (MIF) in human liver and breast tissue. Electrophoresis 1998; 19:2010-3.

43. Liu YC, Nakano T, Elly C, Ishizaka K. Requirement of posttranslational modifications for the generation of biologic activity of glycosylation-inhibiting factor. Proc Natl Acad Sci USA 1994;91:11227-31.

44. Turner J M, Brodsky M H, Irving B A, Levin S D, Perlmutter R M, Littman D R. Interaction of the unique N-terminal region of tyrosine kinase p561ck with cytoplasmic domains of CD4 and CD8 is mediated by cysteine motifs. Cell 1990;60:755-65.

45. Frankel A D, Bredt D S, Pabo C O. Tat protein from human immunodeficiency virus forms a metal-linked dimer. Science 1988;240:70-3.

46. Sowdhamini R, Srinivasan N, Shoichet B, Santi D V, Ramakrishnan C, Balaram P. Stereochemical modeling of disulfide bridges. Criteria for introduction into proteins by site-directed mutagenesis. Protein Eng 1989;3:95-103.

47. Bernhagen, J., Kapumiotu, A., Stoeva, S., Voelter, W., and Bucala, R. Conformational and disulfide bond analysis of macrophage migration inhibitory factor (MIF). Maia, H. L. S. 572-573. 1994. Leiden, The Netherlands, ESCOM. Peptides.

48. Nakano T, Watarai H, Liu Y C, Oyama Y, Mikayama T, Ishizaka K. Conversion of inactive glycosylation inhibiting factor to bioactive derivatives by modification of a SH group. Proc Natl Acad Sci USA 1997;94:202-7.

49. Gething M J, Sambrook J. Protein folding in the cell. Nature 1992;355:33-45.

50. Andrei C. Dazzi C, Lotti L, Torrisi MR, Chimini G, Rubartelli A. The secretary route of the leaderless protein interleukin 1 beta involves exocytosis of endolysosome-related vesicles. Mol Biol Cell 1999; 10: 1463-75.

51. Rubartelli A, Bajetto A, Allavena G, Woliman E, Sitia R. Secretion of thioredoxin by normal and neoplastic cells through a leaderless secretory pathway. J Biol Chem. 1992;267:24161-4.

52. Onodera S, Suzuki K, Matsuno T, Kaneda K, Takagi M, Nishihira J. Macrophage migration inhibitory factor induces phagocytosis of foreign particles by macrophages in autocrine and paracrine fashion. Immunology 1997;92:131-7.

53. Martiney J A, Sherry B, Metz C N, Espinoza M, Ferrer A S, Calandra T et al. Macrophage migration inhibitory factor release by macrophages after ingestion of Plasmodium chabaudi-infected erythrocytes: possible role in the pathogenesis of malarial anemia. Infect.Immun. 2000;68:2259-67.

54. Juttner S, Bernhagen J, Metz C N, Rollinghoff M, Bucala R, Gessner A. Migration inhibitory factor induces killing of Leishmania major by macrophages: dependence on reactive nitrogen intermediates and endogenous TNF-alpha. J Immunol. 1998; 161:2383-90.

55. Li H, Llera A, Mariuzza R A. Structure-function studies of T-cell receptor-superantigen interactions. Immunol Rev. 1998; 163:177-86.

56. Weiser V Y, Temple P A, Witek-Giannotti J S, Remold H G, Clark S C, David J R. Molecular cloning of a cDNA encoding a human macrophage migration inhibitory factor. Proc Natl Acad Sci USA 1989;86:7522-6.

57. Karima R, Matsumoto S, Higashi H, Matsushima K. The molecular pathogenesis of endotoxic shock and organ failure. Mol Med Today 1999;5:123-32.

58. Glauser M P, Zanetti G, Baumgartner J D, Cohen J. Septic shock: pathogenesis. Lancet 1991;338:732-6.

59. Abraham E. Why immunomodulatory therapies have not worked in sepsis. Intensive Care Med 1999;25:556-66.

60. Calandra T, Bucala R. Macrophage migration inhibitory factor: a counter-regulator of glucocorticoid action and critical mediator of septic shock. J Inflamm. 1995;47:39-51.

61. Tracey K J, Beutler B, Lowry S F, Merryweather J, Wolpe S, Milsuk I W et al. Shock and tissue injury induced by recombinant human cachectin. Science 1986;234:470-4.

62. Okusawa S, Gelfand J A, Ikejima T, Connolly R I, Dinarello C A. Interleukin 1 induces a shock-like state in rabbits. Synergism with tumor necrosis factor and the effect of cyclooxygenase inhibition. J Clin Invest 1988;81:1162-72.

63. Calandra T, Echtenacher B, Roy D L, Pugin J, Metz C N, Hultner L et al. Protection from septic shock by neutralization of macrophage migration inhibitory factor. Nat.Med 2000;6: 164-70.

64. Echtenacher B, Falk W, Mannel D N, Krammer P H. Requirement of endogenous tumor necrosis factor/cachectin for recovery from experimental peritonitis. J Immunol 1990; 145:3762-6.

65. Heumann D, Le Roy D, Zanetti G, Eugster H P, Ryffel B, Hahne M et al. Contribution of TNF/TNF receptor and of Fas ligand to toxicity in murine models of endotoxemia and bacterial peritonitis. J Inflamm. 1995;47:173-9.

66. Mikulowska A, Metz C N, Bucala R, Holmdahl R. Macrophage migration inhibitory factor is involved in the pathogenesis of collagen type II-induced arthritis in mice. J Immunol. 1997; 158:5514-7.

67. Lan H Y, Bacher M, Yang N, Mu W, Nikolic-Paterson D J, Metz C et al. The pathogenic role of macrophage migration inhibitory factor in immunologically induced kidney disease in the rat. J Exp Med 1997;185:1455-65.

68. Lan H Y, Mu W, Yang N, Meinhardt A, Nikolic-Paterson D J, Ng Y Y et al. De Novo renal expression of macrophage migration inhibitory factor during the development of rat crescentic glomerulonephritis. Am.J Pathol. 1996; 149:1119-27.

69. Calandra T, Bemhagen J, Metz C N, Spiegel L A, Bacher M. Donnelly T et al. MIF as a glucocorticoid-induced modulator of cytokine production. Nature 1995;377:68-71.

70. Daun J M, Cannon J G. Macrophage migration inhibitory factor antagonizes hydrocortisone-induced increases in cytosolic IkappaBalpha. Am.J Physiol Regul. Integr. Comp Physiol 2000;279:R1043-R1049.

71. Mitchell R A, Metz C N, Peng T, Bucala R. Sustained mitogen-activated protein kinase (MAPK) and cytoplasmic phospholipase A2 activation by macrophage migration inhibitory factor (MIF). Regulatory role in cell proliferation and glucocorticoid action. J Biol Chem. 1999;274:18100-6.

72. Baeuerle P A, Baltimore D. NF-kappa B: ten years after. Cell 1996;87: 13-20.

73. Verma I M, Stevenson J K, Schwarz E M, Van Antwerp D, Miyamoto S. Rel/NF-kappa B/I kappa B family: intimate tales of association and dissociation. Genes Dev 1995;9:2723-35.

74. Auphan N, Didonato J A, Rosette C, Heimberg A, Karin M. Immunosuppression by glucocorticoids: inhibition of NF-kappa B activity through induction of I kappa B synthesis. Science 1995;270:286-90.

75. Scheinman R I, Cogswell P C, Lofquist A K, Baldwin A S, Jr. Role of transcriptional activation of I kappa B alpha in mediation of immunosuppression by glucocorticoids. Science 1995;270:283-6.

76. Gottlicher M, Heck S, Herrlich P. Transcriptional cross-talk, the second mode of steroid hormone receptor action. J Mol Med 1998;76:480-9.

77. Cole T J, Blendy J A, Monaghan A P, Krieglstein K, Schmid W, Aguzzi A et al. Targeted disruption of the glucocorticoid receptor gene blocks adrenergic chromaffin cell development and severely retards lung maturation. Genes Dev 1995;9:1608-21.

78. Kellendonk C, Tronche F, Reichardt H M, Schutz G. Mutagenesis of the glucocorticoid receptor in mice. J Steroid Biochem.Mol Biol 1999;69:253-9.

79. Kleemann R, Hausser A, Geiger G, Mischke R, Burger-Kentischer A, Flieger O et al. Intracellular action of the cytokine MIF to modulate AP-1 activity and the cell cycle through Jab 1. Nature 2000;408:211-6.

80. Lanahan A, Williams J B, Sanders L K, Nathans D. Growth factor-induced delayed early response genes. Mol Cell Biol 1992; 12:3919-29.

81. Wistow G J, Shaughnessy M P, Lee D C, Hodin J, Zelenka P S. A macrophage migration inhibitory factor is expressed in the differentiating cells of the eye lens. Proc Natl Acad Sci U S A 1993;90:1272-5.

82. Chesney J, Metz C, Bacher M, Peng T, Meinhardt A, Bucala R. An essential role for macrophage migration inhibitory factor (MIF) in angiogenesis and the growth of a murine lymphoma. Mol Med 1999;5:181-91.

83. Yao H, York R D, Misra-Press A, Carr D W, Stork P J. The cyclic adenosine monophosphate-dependent protein kinase (PKA) is required for the sustained activation of mitogen-activated kinases and gene expression by nerve growth factor. J Biol Chem. 1998;273:8240-7.

84. Roovers K, Davey G, Zhu X, Bottazzi M E, Assoian R K. Alpha5beta1 integrin controls cyclin D1 expression by sustaining mitogen-activated protein kinase activity in growth factor-treated cells. Mol Biol Cell 1999; 10:3197-204.

85. Edwards A S, Scott J D. A-kinase anchoring proteins: protein kinase A and beyond. Curr. Opin. Cell Biol 2000; 12:217-21.

86. Marais R, Wynne J, Treisman R. The SRF accessory protein Elk-1 contains a growth factor-regulated transcriptional activation domain. Cell 1993;73:381-93.

87. Gottschalk A R, Quintans J. Apoptosis in B lymphocytes: the WEHI-231 perspective. Immunol. Cell Biol 1995;73:8-16.

88. Sherr C J. Mammalian GI cyclins. Cell 1993;73:1059-65.

89. Sherr C J, Roberts J M. CDK inhibitors: positive and negative regulators of G1-phase progression. Genes Dev 1999; 13:1501-12.

90. Han H, Nomura T, Honjo T, Tsubata T. Differential modulation of cyclin-dependent kinase inhibitor p27Kip 1 by negative signaling via the antigen receptor of B cells and positive signaling via CD40. Eur J Immunol 1996;26:2425-32.

91. Maltzman W, Czyzyk L. UV irradiation stimulates levels of p53 cellular tumor antigen in nontransformed mouse cells. Mol Cell Biol 1984;4:1689-94.

92. Kastan M B, Onyekwere O, Sidransky D, Vogelstein B, Craig R W. Participation of p53 protein in the cellular response to DNA damage. Cancer Res 1991;51:6304-11.

93. Yonish-Rouach E, Resnitzky D. Lotem J, Sachs L, Kimchi A, Oren M. Wild-type p53 induces apoptosis of myeloid leukaemic cells that is inhibited by interleukin-6. Nature 1991;352:345-7.

94. Kuerbitz S J, Plunkett B S, Walsh W V, Kastan M B. Wild-type p53 is a cell cycle checkpoint determinant following irradiation. Proc Natl Acad Sci USA 1992;89:7491-5.

95. Clarke A R, Purdie C A, Harrison D J, Morris R G, Bird C C, Hooper M L et al. Thymocyte apoptosis induced by p53-dependent and independent pathways. Nature 1993;362:849-52.

96. Lowe S W, Schmitt E M, Smith S W, Osbome B A, Jacks T. p53 is required for radiation-induced apoptosis in mouse thymocytes. Nature 1993;362:847-9.

97. Lane D P. Cancer. p53, guardian of the genome. Nature 1992;358:15-6.

98. Griffiths S D, Clarke A R, Healy L E, Ross G, Ford A M, Hooper M L et al. Absence of p53 permits propagation of mutant cells following genotoxic damage. Oncogene 1997; 14:523-31.

99. Page L J, Darmon A J, Ueliner R, Griffiths G M. L is for lytic granules: lysosonies that kill. Biochim.Biophys.Acta 1998;1401:146-56.

100. Jenkins J R, Rudge K, Currie G A. Cellular immortalization by a cDNA clone encoding the transformation-associated phosphoprotein p53. Nature 1984;312:651-4.

101. Shay J W, Pereira-Smith O M, Wright W E. A role for both RB and p53 in the regulation of human cellular senescence. Exp Cell Res 1991;196:33-9.

102. Harvey M, Sands A T, Weiss R S, Hegi M E, Wiseman R W, Pantazis P et al. In vitro growth characteristics of embryo fibroblasts isolated from p53-deficient mice. Oncogene 1993;8:2457-67.

103. Moll U M, Riou G, Levine A J. Two distinct mechanisms alter p53 in breast cancer: mutation and nuclear exclusion. Proc Natl Acad Sci USA 1992;89:7262-6.

104. Moll U M, LAQuaglia M, Benard J, Riou G. Wild-type p53 protein undergoes cytoplasmic sequestration in undifferentiated neuroblastomas but not in differentiated tumors. Proc Natl Acad Sci USA 1995;92:4407-11.

105. Maestro R, Gloghini A, Doglioni C, Gasparotto D, Vukosavljevic T, De R, V et al. MDM2 overexpression does not account for stabilization of wild-type p53 protein in non-Hodgkin's lymphomas. Blood 1995;85:3239-46.

106. Hudson J D, Shoaibi M A, Maestro R, Carnero A, Hannon G J, Beach D H. A proinflammatory cytokine inhibits p53 tumor suppressor activity. J Exp Med 1999; 190:1375-82.

107. MacMicking J, Xie Q W, Nathan C. Nitric oxide and macrophage function. Annu.Rev.Immunol 1997; 15:323-50.

108. Weitberg A B, Weitzman S A, Destrempes M4, Latt S A, Stossel T P. Stimulated human phagocytes produce cytogenetic changes in cultured mammalian cells. N Engl J Med 1983;308:26-30.

109. Weitzman S A, Weitberg A B, Clark E P, Stossel T P. Phagocytes as carcinogens: malignant transformation produced by human neutrophils. Science 1985;227:1231-3.

110. Krishna M, Woda B, Savas L, Baker S, Banner B. Expression of p53 antigen in inflamed and regenerated mucosa in ulcerative colitis and Crohn's disease. Mod.Pathol 1995;8:654-7.

111. Hibi K, Mitomi H, Koizumi W, Tanabe S, Saigenji K, Okayasu I. Enhanced cellular proliferation and p53 accumulation in gastric mucosa chronically infected with Helicobacter pylori. Am J Clin Pathol 1997; 108:26-34.

112. Wotherspoon A C. Helicobacter pylori infection and gastric lymphoma. Br Med Bull. 1998;54:79-85.

113. Rosenthal G J, Simeonova P, Corsini E. Asbestos toxicity: an immunologic perspective. Rev.Environ.Health 1999;14: 11-20.

114. Angel P, Karin M. The role of Jun, Fos and the AP-1 complex in cell-proliferation and transformation. Biochim.Biophys.Acta 1991; 1072:129-57.

115. Woodgett J R. Fos and jun: two into one will go. Semin.Cancer Biol 1990;1:389-97.

116. Almendral J M, Sommer D, Macdonald-Bravo H, Burckhardt J, Perera J, Bravo R. Complexity of the early genetic response to growth factors in mouse fibroblasts. Mol Cell Biol 1988;8:2140-8.

117. Lin A, Smeal T, Binetruy B, Deng T, Chambard J C, Karin M. Control of AP-1 activity by signal transduction cascades. Adv Second Messenger Phosphoprotein Res 1993;28:255-60.

118. Claret F X, Hibi M, Dhut S, Toda T, Karin M. A new group of conserved coactivators that increase the specificity of AP-1 transcription factors. Nature 1996;383:453-7.

119. Vives E, Brodin P, Lebleu B. A truncated HIV-1 Tat protein basic domain rapidly translocates through the plasma membrane and accumulates in the cell nucleus. J Biol Chem. 1997;272: 16010-7.

120. Blocki F A, Ellis L B, Wackett L P. MIF protein are theta-class glutathione S-transferase homologs. Protein Sci 1993;2:2095-102.

121. Swope M D, Sun H W, Klockow B, Blake P, Lolis E. Macrophage migration inhibitory factor interactions with glutathione and S-hexylglutathione. J Biol Chem. 1998;273:14877-84.

122. Zhang M, Aman P, Grubb A, Panagopoulos 1, Hindemith A, Rosengren E et al. Cloning and sequencing of a cDNA encoding rat D-dopachrome tautomerase. FEBS Lett. 1995;373:203-6.

123. Odh G, Hindemith A, Rosengren A M, Rosengren E, Rorsman H. Isolation of a new tautomerase monitored by the conversion of D-dopachrome to 5,6-dihydroxyindole. Biochem.Biophys.Res Commun. 1993; 197:619-24.

124. Sugimoto H, Taniguchi M, Nakagawa A, Tanaka I, Suzuki M, Nishihira J. Crystallization and preliminary X-ray analysis of human D-dopachrome tautomerase. J Struct.Biol 1997; 120:105-8.

125. Kuriyama T, Fujinaga M, Koda T, Nishihira J. Cloning of the mouse gene for D-dopachrome tautomerase. Biochim.Biophys.Acta 1998; 1388:506-12.

126. Bjork P, Aman P, Hindemith A, Odh G, Jacobsson L, Rosengren E et al. A new enzyme activity in human blood cells and isolation of the responsible protein (D-dopachrome tautomerase) from erythrocytes. Eur J Haematol 1996;57:254-6.

127. Subramanya H S, Roper D I, Dauter Z, Dodson E J, Davies G J, Wilson K S et al. Enzymatic ketonization of 2-hydroxymuconate: specificity and mechanism investigated by the crystal structures of two isomerases. Biochemistry 1996;35:792-802.

128. Sugimoto H, Taniguchi M, Nakagawa A, Tanaka I, Suzuki M, Nishihira J. Crystal structure of human D-dopachrome tautomerase, a homologue of macrophage migration inhibitory factor, at 1.54 A resolution. Biochemistry 1999;38:3268-79.

129. Rosengren E, Bucala R, Aman P, Jacobsson L. Odh G, Metz C N et al. The immunoregulatory mediator macrophage migration inhibitory factor (MIF) catalyzes a tautomerization reaction. Mol Med 1996;2:143-9.

130. Rosengren E, Aman P, Thelin S, Hansson C, Ahlfors S, Bjork P et al. The macrophage migration inhibitory factor MIF is a phenylpyruvate tautomerase. FEBS Lett. 1997;417:85-8.

131. Deutsch J C. Determination of p-hydroxyphenylpyruvate, p-hydroxyphenyllactate and tyrosine in normal human plasma by gas chromatography-mass spectrometry isotope-dilution assay. J Chromatogr.BBiomed.Sci Appl. 1997;690:1-6.

132. Stivers J T, Abeygunawardana C, Mildvan A S, Hajipour G, Whitman C P, Chen L H. Catalytic role of the amino-terminal proline in 4-oxalocrotonate tautomerase: affinity labeling and heteronuclear NMR studies. Biochemistry 1996;35:803-13.

133. Stivers J T, Abeygunawardana C, Mildvan A S, Hajipour G, Whitman C P. 4-Oxalocrotonate tautomerase: pH dependence of catalysis and pKa values of active site residues. Biochemistry 1996;35:814-23.

134. Fitzgerald M C, Chemushevich I, Standing K G, Whitman C P, Kent S B. Probing the oligomeric structure of an enzyme by electrospray ionization time-of-flight mass spectrometry. Proc Natl Acad Sci USA 1996;93:6851-6.

135. Stivers J T, Abeygunawardana C, Whitman C P, Mildvan A S. 4-Oxalocrotonate tautomerase, a 41 kDa homohexamer: backbone and side-chain resonance assignments, solution secondary structure, and locationωof active site residues by heteronuclear NMR spectroscopy. Protein Sci 1996;5:729-41.

136. Stivers J T, Abeygunawardana C, Mildvan A S. 15N NMR relaxation studies of free and inhibitor-bound 4-oxalocrotonate tautomerase: backbone dynamics and entropy changes of an enzyme upon inhibitor binding. Biochemistry 1996;35:16036-47.

137. Taylor A B, Czerwinski R M, Johnson W H, Whitman C P, Hackert M L. Crystal structure of 4-oxalocrotonate tautomerase inactivated by 2-oxo-3-pentynoate at 2.4 A resolution: analysis and implications for the mechanism of inactivation and catalysis. Biochemistry 1998;37: 14692-700.

138. Swope M, Sun H W, Blake P R, Lolis E. Direct link between cytokine activity and a catalytic site for macrophage migration inhibitory factor. EMBO J. 1998; 17:3534-41.

139. Bendrat K, Al Abed Y, Callaway D J, Peng T, Calandra T, Metz C N et al. Biochemical and mutational investigations of the enzymatic activity of macrophage migration inhibitory factor. Biochemistry 1997;36: 15356-62.

140. Johnson W H, Czerwinski R M, Stamps S L, Whitman C P. A kinetic and stereochemical investigation of the role of lysine-32 in the phenylpyruvate tautomerase activity catalyzed by macrophage migration inhibitory factor. Biochemistry 1999;38:16024-33.

141. Lubetsky J B, Swope M, Dealwis C, Blake P, Lolis E. Pro-1 of macrophage migration inhibitory factor functions as a catalytic base in the phenylpyruvate tautomerase activity. Biochemistry 1999;38:7346-54.

142. Soares T, Goodsell D, Ferreira R, Olson A J, Briggs J M. Ionization state and molecular docking studies for the macrophage migration inhibitory factor: the role of lysine 32 in the catalytic mechanism. J Mol Recognit. 2000; 13:146-56.

143. Sun H W, Swope M, Cinquina C, Bedarkar S, Bemhagen J, Bucala R et al. The subunit structure of human macrophage migration inhibitory factor: evidence for a trimer. Protein Eng 1996;9:631-5.

144. Hermanowski-Vosatka A, Mundt S S, Ayala J M, Goya] S, Hanlon W A, Czerwinski R M et al. Enzymatically inactive macrophage migration inhibitory factor inhibits monocyte chemotaxis and random migration. Biochemistry 1999;38: 12841-9.

145. Takahashi N, Creighton T E. On the reactivity and ionization of the active site cysteine residues of Escherichia coli thioredoxin. Biochemistry 1996;35:8342-53.

146. Puig A, Lyles M M, Noiva R, Gilbert H F. The role of the thiol/disulfide centers and peptide binding site in the chaperone and anti-chaperone activities of protein disulfide isomerase. J. Biol. Chem. 1994;269:19128-35.

147. Zapun A, Cooper L, Creighton T E. Replacement of the active-site cysteine residues of DsbA, a protein required for disulfide bond formation in vivo. Biochemistry 1994;33:1907-14.

148. Chandler M L, Varandani P T. Insulin degradation. XV. Use of different assay methods for the study of mechanism of action of glutathione-insulin transhydrogenase. Biochim.Biophys.Acta 1975;397:307-17.

149. Holmgren A. Thioredoxin catalyzes the reduction of insulin disulfides by dithiothreitol and dihydrolipoamide. J Biol Chem. 1979;254:9627-32.

150. Kleemann R, Kapurniotu A, Frank R W, Gessner A, Mischke R, Flieger O et al. Disulfide analysis reveals a role for macrophage migration inhibitory factor (MIF) as thiol-protein oxidoreductasc. J Mol Biol 1998;280:85-102.

151. Kleemann R, Mischke R, Kapurniotu A, Brunner H, Bernhagen J. Specific reduction of insulin disulfides by macrophage migration inhibitory factor (MIF) with glutathione and dihydrolipoamide: potential role in cellular redox processes. FEBS Lett. 1998;430: 191-6.

152. Pastrana D V, Raghavan N, FitzGerald P, Eisinger S W, Metz C, Bucala R et al. Filarial nematode parasites secrete a homologue of the human cytokine macrophage migration inhibitory factor. Infect.Immun. 1998;66:5955-63.

153. Sulston J E, Horvitz H R. Post-embryonic cell lineages of the nematode, Caenorhabditis elegans. Dev Biol 1977;56:110-56.

154. Kenyon C J, Austin J, Costa M, Cowing D W, Harris J M, Honigberg L et al. The dance of the Hox genes: patterning the anteroposterior body axis of Caenorhabditis elegans. Cold Spring Harb.Symp.Quant.Biol 1997;62:293-305.

155. Komfeld K. Vulval development in Caenorhabditis elegans. Trends Genet 1997; 13:55-61.

156. Marin I, Baker B S. The evolutionary dynamics of sex determination. Science 1998;281:1990-4.

157. MacLeod A R, Karn J, Brenner S. Molecular analysis of the unc-54 myosin heavy-chain gene of Caenorhabditis elegans. Nature 1981;291:386-90.

158. Metzstein M M, Stanfield G M, Horvitz H R. Genetics of programmed cell death in C. elegans: past, present and future. Trends Genet 1998; 14:410-6.

159. Hekimi S, Lakowski B, Barnes T M, Ewbank J J. Molecular genetics of life span in C. elegans: how much does it teach us? Trends Genet 1998; 14:14-20.

160. Genome sequence of the nematode C. elegans: a platform for investigating biology. The C. elegans Sequencing Consortium. Science 1998;282:2012-8.

161. Fire A, Xu S, Montgomery M K, Kostas S A, Driver S E, Mello C C. Potent and specific genetic interference by double-stranded RNA in Caenorhabditis elegans. Nature 1998;391:806-11.

162. Kennerdell J R, Carthew R W. Heritable gene silencing in Drosophila using double-stranded RNA. Nat.Biotechnol. 2000;18:896-8.

163. Ngo H, Tschudi C, Gull K, Ullu E. Double-stranded RNA induces mRNA degradation in Trypanosoma brucei. Proc Natl Acad Sci USA 1998;95:14687-92.

164. Waterhouse P M, Graham M W, Wang M B. Virus resistance and gene silencing in plants can be induced by simultaneous expression of sense and antisense RNA. Proc Natl Acad Sci USA 1998;95:13959-64.

165. Tabara H, Grishok A, Mello C C. RNAi in C. elegans: soaking in the genome sequence. Science 1998;282:430-1.

166. Timmons L, Fire A. Specific interference by ingested dsRNA. Nature 1998;395:854.

167. Montgomery M K, Xu S, Fire A. RNA as a target of double-stranded RNA-mediated genetic interference in Caenorhabditis elegans. Proc Natl Acad Sci USA 1998;95:15502-7.

168. Ketting R F, Haverkamp T H, van Luenen H G, Plasterk R H. Mut-7 of C. elegans, required for transposon silencing and RNA interference, is a homolog of Werner syndrome helicase and RNaseD. Cell 1999;99:133-41.

169. Smardon A, Spoerke J M, Stacey S C, Klein M E, Mackin N, Maine E M. EGO-1 is related to RNA-directed RNA polymerase and functions in germ-line development and RNA interference in C. elegans. Curr.Biol 2000;10: 169-78.

170. Tabara H, Sarkissian M, Kelly W G, Fleenor J, Grishok A, Timmons L et al., The rde-1 gene, RNA interference, and transposon silencing in C. elegans. Cell 1999;99: 123-32.

171. Zamore P D, Tuschl T, Sharp P A, Bartel D P. RNAi: double-stranded RNA directs the ATP—dependent cleavage of mRNA at 21 to 23 nucleotide intervals. Cell 2000;101:25-33.

172. Domeier M E, Morse D P, Knight S W, Portereiko M, Bass B L, Mango S E. A link between RNA interference and nonsense-mediated decay in Caenorhabditis elegans. Science 2000;289: 1928-31.

173. Hodgkin J, Papp A, Pulak R, Ambros V, Anderson P. A new kind of informational suppression in the nematode Caenorhabditis elegans4. Genetics 1989; 123:301-13.

174. Cali B M, Kuchma S L, Latham J, Anderson P. smg-7 is required for mRNA surveillance in Caenorhabditis elegans. Genetics 1999; 151:605-16.

175. Folger K R, Wong E A, Wahl G, Capeccbi M R. Patterns of integration of DNA microinjected into cultured mammalian cells: evidence for homologous recombination between injected plasmid DNA molecules. Mol Cell Biol 1982;2:1372-87.

176. Wong E A, Capecchi M R. Homologous recombination between coinjected DNA sequences peaks in early to mid-S phase. Mol Cell Biol 1987;7:2294-5.

177. Ayares D, Chekuri L, Song K Y, Kucherlapati R. Sequence homology requirements for intermolecular recombination in mammalian cells. Proc Natl Acad Sci USA 1986;83:5199-203.

178. Kucherlapati R S, Eves E M, Song KY, Morse BS, Smithies 0. Homologous recombination between plasmids in mammalian cells can be enhanced by treatment of input DNA. Proc Natl Acad Sci USA 1984;81:3153-7.

179. Evans M J, Kaufman M H. Establishment in culture of pluripotential cells from mouse embryos. Nature 1981;292:154-6.

180. Bradley A, Evans M, Kaufman M H, Robertson E. Formation of germ-line chimaeras from embryo-derived teratocarcinoma cell lines. Nature 1984;309:255-6.

181. Brinster R L, Avarbock M R. Germline transmission of donor haplotype following spermatogonial transplantation. Proc Natl Acad Sci USA 1994;91:11303-7.

182. Nagano M, Brinster C J, Orwig K E, Ryu B Y, Avarbock M R, Brinster R L. From the Cover: Transgenic mice produced by retroviral transduction of male germ-line stem cells. Proc Natl Acad Sci USA 2001;98:13090-5.

183. Hasty P, Abuin A, Bradley A. Gene targeting, principles and practice in mammalian cells. In: Joyner A L. Gene Targeting. A practical Approach. New York: Oxford University Press, 2000:1-34.

184. Fiering S, Epner E, Robinson K, Zhuang Y, Telling A, Hu M et al. Targeted deletion of 5′HS2 of the murine beta-globin LCR reveals that it is not essential for proper regulation of the beta-globin locus. Genes Dev 1995;9:2203-13.

185. Xu Y, Davidson L, Alt F W, Baltimore D. Deletion of the Ig kappa light chain intronic enhancer/matrix attachment region impairs but does not abolish V kappa J kappa rearrangement. Immunity. 1996;4:377-85.

186. Lerner A, D'Adamio L, Diener A C, Clayton L K, Reinherz E L. CD3 zeta/eta/theta locus is colinear with and transcribed antisense to the gene encoding the transcription factor Oct-1. J lmmunol 1993;151:3152-62.

187. Koyasu S, Hussey R E, Clayton L K, Lerner A, Pedersen R, Delany-Heiken P et al. Targeted disruption within the CD3 zeta/eta/phi/Oct-1 locus in mouse. EMBO J. 1994;13:784-97.

188. Ohno H, Goto S, Taki S, Shirasawa T, Nakano H, Miyatake S et al. Targeted disruption of the CD3 eta locus causes high lethality in mice: modulation of Oct-I transcription on the opposite strand. EMBO J. 1994; 13:1157-65.

189. Muller U, Cristina N, Li Z W, Wolfer D P, Lipp H P, Rulicke T et al. Behavioral and anatomical deficits in mice homozygous for a modified beta-amyloid precursor protein gene. Cell 1994;79:755-65.

190. Sauer B, Henderson N. Site-specific DNA recombination in mammalian cells by the Cre recombinase of bacteriophage PI. Proc Natl Acad Sci USA 1988;85:5166-70.

191. Rajewsky K, Gu H, Kuhn R. Betz U A, Muller W, Roes J et al. Conditional gene targeting. J Clin Invest 1996;98:600-3.

192. Austin S, Ziese M. Sternberg N. A novel role for site-specific recombination in maintenance of bacterial replicons. Cell 1981;25:729-36.

193. Hochman L, Segev N, Sternberg N, Cohen G. Site-specific recombinational circularization of bacteriophage P1 DNA. Virology 1983; 131:11-7.

194. Hoess R H, Ziese M, Sternberg N. P1 site-specific recombination: nucleotide sequence of the recombining sites. Proc Natl Acad Sci USA 1982;79:3398-402.

195. Hoess R H, Abremski K. Interaction of the bacteriophage P1 recombinase Cre with the recombining site loxP. Proc Natl Acad Sci USA 1984;81:1026-9.

196. Argos P, Landy A, Abremski K. Egan J B, Haggard-Ljungquist E, Hoess R H et al. The integrase family of site-specific recombinases: regional similarities and global diversity. EMBO J. 1986;5:433-40.

197. Sadowski PD. Site-specific genetic recombination: hops, flips, and flops. FASEB J 1993;7:760-7.

198. Hoess R H, Abremski K. Mechanism of strand cleavage and exchange in the Cre-lox site-specific recombination system. J Mol Biol 1985; 181:351-62.

199. Stark W M, Boocock M R, Sherratt D J. Catalysis by site-specific recombinases. Trends Genet 1992;8:432-9.

200. Mack A, Sauer B, Abremski K, Hoess R. Stoichiometry of the Cre recombinase bound to the lox recombining site. Nucleic Acids Res 1992;20:4451-5.

201. Hoess R, Wierzbicki A, Abremski K. Formation of small circular DNA molecules via an in vitro site-specific recombination system. Gene 1985;40:325-9.

202. Kuhn R, Schwenk F, Aguet M, Rajewsky K. Inducible gene targeting in mice. Science 1995;269:1427-9.

203. Rohlmann A, Gotthardt M, Willnow T E, Hammer R E, Herz J. Sustained somatic gene inactivation by viral transfer of Cre recombinase. Nat.Biotechnol. 1996;14:1562-5.

204. Wang Y, Krushel L A, Edelman G M. Targeted DNA recombination in vivo using an adenovirus carrying the cre recombinase gene. Proc Natl Acad Sci USA 1996;93:3932-6.

205. Schorle H, Holtschke T, Hunig T, Schimpl A, Horak I. Development and function of T cells in mice rendered interleukin-2 deficient by gene targeting. Nature 1991;352:621-4.

206. Sadlack B, Merz H, Schorle H, Schimpl A, Feller A C, Horak I. Ulcerative colitis-like disease in mice with a disrupted interleukin-2 gene. Cell 1993;75:253-61.

207. Horak I, Lohler J, Ma A, Smith K A. Interleukin-2 deficient mice: a new model to study autoimmunity and self-tolerance. Immunol Rev. 1995; 148:35-44.

208. De Togni P, Goellner J, Ruddle N H, Streeter P R, Fick A, Mariathasan S et al. Abnormal development of peripheral lymphoid organs in mice deficient in lymphotoxin. Science 1994;264:703-7.

209. Banks T A, Rouse B T, Kerley M K, Blair P J, Godfrey V L, Kuklin N A et al. Lymphotoxin-alpha-deficient mice. Effects on secondary lymphoid organ development and humoral immune responsiveness. J Immunol 1995; 155:1685-93.

210. Stanley E, Lieschke G J, Grail D, Metcalf D, Hodgson G, Gall J A et al. Granulocyte/macrophage colony-stimulating factor-deficient mice show no major perturbation of hematopoiesis but develop a characteristic pulmonary pathology. Proc Natl Acad Sci U S A 1994;91:5592-6.

211. Dranoff G, Crawford A D, Sadelain M, Ream B, Rashid A, Bronson R T et al. Involvement of granulocyte-macrophage colony-stimulating factor in pulmonary homeostasis. Science 1994;264:713-6.

212. Fantuzzi G, Zheng H, Faggioni R, Benigni F, Ghezzi P, Sipe J D et al. Effect of endotoxin in IL-1 beta-deficient mice. J Immunol 1996; 157:291-6.

213. Shull M M, Ormsby I, Kier A B, Pawlowski S, Diebold R J, Yin M et al. Targeted disruption of the mouse transforming growth factor-beta I gene results in multifocal inflammatory disease. Nature 1992;359:693-9.

214. Stewart C L, Kaspar P, Brunet L J, Bhatt H, Gadi I, Kontgen F et al. Blastocyst implantation depends on maternal expression of leukaemia inhibitory factor. Nature 1992;359:76-9.

215. Compton M M, Caron L A, Cidlowski J A. Glucocorticoid action on the immune system. J Steroid Biochem. 1987;27:201-8.

216. Bond W S. Toxic reactions and side effects of glucocorticoids in man. Am.J Hosp.Pharm. 1977;34:479-85.

217. Toft P, Tonnesen E, Svendsen P, Rasmussen J W. Redistribution of lymphocytes after cortisol administration. APMIS 1992; 100: 154-8.

218. Cupps T R, Fauci A S. Corticosteroid-mediated immunoregulation in man. Immunol.Rev. 1982;65:133-55.

219. Adams S, van der Laan L J, Vernon-Wilson E, Renardel d L, Dopp E A, Dijkstra C D et al. Signal-regulatory protein is selectively expressed by myeloid and neuronal cells. J Immunol, 1998;161:1853-9.

220. Mills C M, Marks R. Side effects of topical glucocorticoids. Curr.Probl. Dermatol. 1993;21:122-31.

221. da Silva J A. Sex hormones and glucocorticoids: interactions with the immune system. Ann N Y.Acad Sci 1999;876:102-17.

222. Vacchio M S, Papadopoulos V, Ashwell J D. Steroid production in the thymus: implications for thymocyte selection. J Exp Med 1994; 179:1835-46.

223. Cohen J J, Duke R C. Glucocorticoid activation of a calcium-dependent endonuclease in thymocyte nuclei leads to cell death. J Immunol 1984; 132:38-42.

224. Thompson E B, Thulasi R, Saeed M F, Johnson B H. Glucocorticoid antagonist RU 486 reverses agonist-induced apoptosis and c-myc repression in human leukemic CEM-C7 cells. Ann NY.Acad Sci 1995;761:261-75.

225. Mosmann T. Rapid colorimetric assay for cellular growth and survival: application to proliferation and cytotoxicity assays. J Immunol Methods 1983;65:55-63.

226. Joyce D A, Steer J H, Abraham L J. Glucocorticoid modulation of human monocyte/macrophage function: control of TNF-alpha secretion. Inflamm. Res 1997;46:447-51.

227. Fantuzzi G, Demitri M T, Ghezzi P. Differential effect of glucocorticoids on tumour necrosis factor production in mice: up-regulation by early pretreatment with dexamethasone. Clin Exp Immunol 1994;96:166-9.

228. Nishibira J, Fujinaga M, Kuriyama T, Suzuki M, Sugimoto H, Nakagawa A et al. Molecular cloning of human D-dopachrome tautomerase cDNA: N-terminal proline is essential for enzyme activation. Biochem.Biophys.Res Commun. 1998;243:538-44.

229. Jaworski D C, Jasinskas A, Metz C N, Bucala R, Barbour A G. Identification and characterization of a homologue of the pro-inflammatory cytokine Macrophage Migration Inhibitory Factor in the tick, Amblyomma americanum. Insect Mol Biol 2001; 10:323-31.

230. Suzuki T, Ogata A, Tashiro K, Nagashima K, Tamura M, Nishihira J. Augmented expression of macrophage migration inhibitory factor (MIF) in the telencephalon of the developing rat brain. Brain Res 1999;816:457-62.

231. Lemckert F A, Sedgwick J D, Komer H. Gene targeting in C57B1/6 ES cells. Successful germ line transmission using recipient BALB/c blastocysts developmentally matured in vitro. Nucleic Acids Res 1997;25:917-8.

232. Wada S, Fujimoto S, Mizue Y, Nishihira J. Macrophage migration inhibitory factor in the human ovary: presence in the follicular fluids and production by granulosa cells. Biochem.Mol Biol Int. 1997;41:805-14.

233. Arcuri F, Ricci C, letta F, Cintorino M, Tripodi S A, Cetin I et al. Macrophage Migration Inhibitory Factor in the Human Endometrium: Expression and Localization During the Menstrual Cycle and Early Pregnancy. Biol Reprod. 2001;64:1200-5.

234. Meinhardt A, Bacher M, Wennemuth G, Eickhoff R, Hedger M. Macrophage migration inhibitory factor (MIF) as a paracrine mediator in the interaction of testicular somatic cells. Andrologia 2000;32:46-8.

235. Eickhoff R, Wilhelm B, Renneberg H, Wennemuth G, Bacher M, Linder D et al. Purification and Characterization of Macrophage Migration Inhibitory Factor as a Secretory Protein from Rat Epididymis: Evidences for Alternative Release and Transfer to Spermatozoa. Molecular Medicine 2001;7:27-35.

236. Bozza M, Satoskar A R, Lin G, Lu B, Humbles A A, Gerard C et al. Targeted disruption of migration inhibitory factor gene reveals its critical role in sepsis. J Exp Med 1999; 189:341-6.

237. Honma N, Koseki H, Akasaka T, Nakayama T, Taniguchi M, Serizawa I et al. Deficiency of the macrophage migration inhibitory factor gene has no significant effect on endotoxaemia. Immunology 2000; 100:84-90.

238. Mitchell R A, Bucala R. Tumor growth-promoting properties of macrophage migration inhibitory factor (MIF). Semin. Cancer Biol 2000; 10:359-66.

239. del Vecchio M T, Tripodi S A, Arcuri F, Pergola L, Hako L, Vatti R et al. Macrophage migration inhibitory factor in prostatic adenocarcinoma: correlation with tumor grading and combination endocrine treatment-related changes. Prostate 2000;45:51-7.

240. Petrenko O, ischenko I, Enrietto P J. Characterization of changes in gene expression associated with malignant transformation by the NF-kappaB family member, v-Rel. Oncogene 1997;15:1671-80.

241. Bos J L. ras oncogenes in human cancer: a review. Cancer Res 1989;49:4682-9.

242. Nakamura Y, Gojobori T, Ikemura T. Codon usage tabulated from the international DNA sequence databases; its status 1999. Nucleic Acids Res 1999;27:292.

243. Bunting M, Bernstein K E, Greer J M, Capecchi M R, Thomas K R. Targeting genes for self-excision in the germ line. Genes Dev 1999;13:1524-8

244. Kazmi M A, Sakmar T P, Ostrer H. Mutation of a conserved cysteine in the X-linked cone opsins causes color vision deficiencies by disrupting protein folding and stability. Invest Ophthalmol. Vis.Sci 1997;38:1074-81.

245. Bernhagen J, Calandra T, Cerami A, Bucala R. Macrophage migration inhibitory factor is a neuroendocrine mediator of endotoxaemia. Trends Microbiol. 1994;2:198-201.

246. Leech M, Metz C, Santos L, Peng T, Holdsworth S R, Bucala R et al. Involvement of macrophage migration inhibitory factor in the evolution of rat adjuvant arthritis. Arthritis Rheum. 1998;41:910-7.

247. Yang N, Nikolic-Paterson D J, Ng Y Y, Mu W, Metz C, Bacher M et al. Reversal of established rat crescentic glomerulonephritis by blockade of macrophage migration inhibitory factor (MIF): potential role of MIF in regulating glucocorticoid production. Mol Med 1998;4:413-24.

248. Linzer D I, Wilder E L. Control of proliferin gene expression in serum-stimulated mouse cells. Mol Cell Biol 1987;7:2080-6.

249. Takahashi N, Nishihira J, Sato Y, Kondo M, Ogawa H, Ohshima T et al. Involvement of macrophage migration inhibitory factor (MIF) in the mechanism of tumor cell growth. Mol Med 1998;4:707-14.

250. The Molecular Basis of Cell Cycle and Growth Control. New York: Wiley-Liss, 1999.

251. Mittnacht S. Control of pRB phosphorylation. Curr. Opin. Genet Dev 1998;8:21-7.

252. Dyson N. The regulation of E2F by pRB-family proteins. Genes Dev 1998; 12:2245-62.

253. Frame S, Cohen P. GSK3 takes centre stage more than 20 years after its discovery. Biochem.J 2001;359:1-16.

254. Jacks T, Fazeli A, Schmitt E M, Bronson R T, Goodell M A, Weinberg R A. Effects of an Rb mutation in the mouse. Nature 1992;359:295-300.

255. Nakayama K, Ishida N, Shirane M, Inomata A, Inoue T, Shishido N et al. Mice lacking p27(Kip 1) display increased body size, multiple organ hyperplasia, retinal dysplasia, and pituitary tumors. Cell 1996;85:707-20.

256. Fantl V, Stamp G, Andrews A, Rosewell I, Dickson C. Mice lacking cyclin D1 are small and show defects in eye and mammary gland development. Genes Dev 1995;9:2364-72.

257. Sicinski P, Donaher J L, Parker S B, Li T, Fazeli A, Gardner H et al. Cyclin D1 provides a link between development and oncogenesis in the retina and breast. Cell 1995;82:621-30.

258. Dunker N, Krieglstein K. Targeted mutations of transforming growth factor-beta genes reveal important roles in mouse development and adult homeostasis. Eur J Biochem. 2000;267:6982-8.

259. Jacks T, Remington L, Williams B O, Schmitt E M, Halachmi S, Bronson RT et al. Tumor spectrum analysis in p53-mutant mice. Curr.Biol 1994;4: 1-7.

260. Chen M S, Hurov J, White L S, Woodford-Thomas T, Piwnica-Worms H. Absence of apparent phenotype in mice lacking Cdc25C protein phosphatase. Mol Cell Biol 2001;21:3853-61.

261. Cobrinik D, Lee M H, Hannon G, Mulligan G, Bronson R T, Dyson N et al. Shared role of the pRB-related p130 and p107 proteins in limb development. Genes Dev 1996; 10:1633-44.

262. LeCouter J E, Kablar B, Whyte P F, Ying C, Rudnicki M A. Strain-dependent embryonic lethality in mice lacking the retinoblastoma-related p130 gene. Development 1998; 125:4669-79.

263. Deng C, Zhang P, Harper J W, Elledge S J, Leder P. Mice lacking p21CIP1/WAF1 undergo normal development, but are defective in G1 checkpoint control. Cell 1995;82:675-84.

264. Sharpless N E, Bardeesy N, Lee K H, Carrasco D, Castrillon D H, Aguirre A J et al. Loss of p16Ink4a with retention of p19Arfpredisposes mice to tumorigenesis. Nature 2001;413:86-91.

265. Satoskar A R, Bozza M, Rodriguez S M, Lin G, David J R. Migration-inhibitory factor gene-deficient mice are susceptible to cutaneous Leishmania major infection. Infect.Immun. 2001;69:906-11.

266. LeCouter J E, Kablar B, Hardy W R, Ying C, Megeney L A, May L L et al. Strain-dependent myeloid hyperplasia, growth deficiency, and accelerated cell cycle in mice lacking the Rb-related p107 gene. Mol Cell Biol 1998; 18:7455-65.

267. Liu J P, Baker J, Perkins A S, Robertson E J, Efstratiadis A. Mice carrying null mutations of the genes encoding insulin-like growth factor I (Igf-1) and type 1 IGF receptor (Igflr). Cell 1993;75:59-72.

268. George E L, Georges-Labouesse E N, Patel-King R S, Rayburn H, Hynes R O. Defects in mesoderm, neural tube and vascular development in mouse embryos lacking fibronectin. Development 1993;119:1079-91.

269. Sibilia M, Wagner E F. Strain-dependent epithelial defects in mice lacking the EGF receptor. Science 1995;269:234-8.

270. Threadgill D W, Dlugosz A A, Hansen L A, Tennenbaum T, Lichti U, Yee D et al. Targeted disruption of mouse EGF receptor: effect of genetic background on mutant phenotype. Science 1995;269:230-4.

271. Rozmahel R, Wilschanski M, Matin A, Plyte S, Oliver M, Auerbach W et al. Modulation of disease severity in cystic fibrosis transmembrane conductance regulator deficient mice by a secondary genetic factor. Nat. Genet 1996; 12:280-7.

272. Bonyadi M, Rusholme S A, Cousins F M, Su H C, Biron C A, Farrall M et al. Mapping of a major genetic modifier of embryonic lethality in TGF beta I knockout mice. Nat.Genet 1997; 15:207-11.

273. Proetzel G, Pawlowski S A, Wiles M V, Yin M, Boivin G P, Howles P N et al. Transforming growth factor-beta 3 is required for secondary palate fusion. Nat. Genet 1995; 11:409-14.

274. Rohrer D K, Desai K H, Jasper J R, Stevens M E, Regula D P, Jr., Barsh G S et al. Targeted disruption of the mouse beta 1-adrenergic receptor gene: developmental and cardiovascular effects. Proc Natl Acad Sci USA 1996;93:7375-80.

275. Yaniasaki L, Bronson R, Williams B O, Dyson N J, Harlow E, Jacks T. Loss of E2F-1 reduces tumorigenesis and extends the lifespan of Rbl (+/−) mice. Nat.Genet 1998;18:360-4.

276. Zhang S L, DuBois W, Ramsay E S, Bliskovski V, Morse H C, III, Taddesse-Heath L et al. Efficiency alleles of the Pctr I modifier locus for plasmacytoma susceptibility. Mol Cell Biol 2001;21:310-8.

277. Bourne H R, Sanders D A, McCormick F. The GTPase superfamily: conserved structure and molecular mechanism. Nature 1991;349:117-27.

278. Oldham S M, Clark G J, Gangarosa L M, Coffey R J, Jr., Der C J. Activation of the Raf-1/MAP kinase cascade is not sufficient for Ras transformation of RIE-1 epithelial cells. Proc Natl Acad Sci USA 1996;93:6924-8.

279. Yatani A, Okabe K, Polakis P, Halenbeck R, McCormick F, Brown A M. ras p21 and GAP inhibit coupling of muscarinic receptors to atrial K+ channels. Cell 1990;61:769-76.

280. Feig L A, Urano T, Cantor S. Evidence for a Ras/Ral signaling cascade. Trends Biochem.Sci 1996;21:438-41.

281. Van Aelst L, White M A, Wigler M H. Ras partners. Cold Spring Harb.Symp. Quant.Biol 1994;59:181-6.

282. Kuriyama M, Harada N, Kuroda S, Yamamoto T, Nakafuku M, lwamatsu A et al. Identification of AF-6 and canoe as putative targets for Ras. J Biol Chem. 1996;271:607-10.

283. Han L, Wong D, Dhaka A, Afar D, White M, Xie W et al. Protein binding and signaling properties of RIN 1 suggest a unique effector function. Proc Natl Acad Sci USA 1997;94:4954-9.

284. Rodriguez-Viciana P, Wame P H, Dhand R, Vanhaesebroeck B, Gout I, Fry M J et al. Phosphatidylinositol-3-OH kinase as a direct target of Ras. Nature 1994;370:527-32.

285. Vojtek A B, Der C J. Increasing complexity of the Ras signaling pathway. J Biol Chem. 1998;273:19925-8.

286. Downward J. Ras signalling and apoptosis. Curr.Opin.GenetDev 1998;8:49-54.

287. Whyte P, Buchkovich K J, Horowitz J M, Friend S H, Raybuck M, Weinberg R A et al. Association between an oncogene and an anti-oncogene: the adenovirus EIA proteins bind to the retinoblastoma gene product. Nature 1988;334: 124-9.

288. Bagchi S, Raychaudhuri P, Nevins J R. Adenovirus E1A proteins can dissociate heteromeric complexes involving the E2F transcription factor: a novel mechanism for E1A trans-activation. Cell 1990;62:659-69.

289. Chellappan S, Kraus V B, Kroger B, Munger K, Howley P M, Phelps W C et al. Adenovirus E IA, simian virus 40 tumor antigen, and human papillomavirus E7 protein share the capacity to disrupt the interaction between transcription factor E2F and the retinoblastoma gene product. Proc Natl Acad Sci USA 1992;89:4549-53.

290. Giles R H, Peters D J, Breuning M H. Conjunction dysfunction: CBP/p300 in human disease. Trends Genet 1998;14:178-83.

291. Henriksson M, Luscher B. Proteins of the Myc network: essential regulators of cell growth and differentiation. Adv Cancer Res 1996;68: 109-82.

292. Amati B, Alevizopoulos K, Vlach J. Myc and the cell cycle. Front Biosci. 1998;3:D250-D268.

293. Alevizopoulos K, Vlach J, Hennecke S, Amati B. Cyclin E and c-Myc promote cell proliferation in the presence of p 161NK4a and of hypophosphorylated retinoblastoma family proteins. EMBO J. 1997;16:5322-33.

294. Smeal T, Binetruy B, Mercola D A, Birrer M. Karin M. Oncogenic and transcriptional cooperation with Ha-Ras requires phosphorylation of c-Jun on serines 63 and 73. Nature 1991;354:494-6.

295. Smeal T, Binetruy B, Mercola D, Grover-Bardwick A, Heidecker G, Rapp U R et al. Oncoprotein-mediated signalling cascade stimulates c-Jun activity by phosphorylation of serines 63 and 73. Mol Cell Biol 1992;12:3507-13.

296. Lloyd A, Yancheva N, Wasylyk B. Transformation suppressor activity of a Jun transcription factor lacking its activation domain. Nature 1991;352:635-8.

297. Bos T J, Monteclaro F S, Mitsunobu F, Ball A R, Jr., Chang C H, Nishimura T et al. Efficient transformation of chicken embryo fibroblasts by c-Jun requires structural modification in coding and noncoding sequences. Genes Dev 1990;4:1677-87.

298. Johnson R, Spiegelman B, Hanahan D, Wisdom R. Cellular transformation and malignancy induced by ras require c-jun. Mol Cell Biol 1996; 16:4504-11.

299. Cheng M, Olivier P, Diehl J A, Fero M, Roussel M F, Roberts J M et al. The p21(Cip1) and p27(Kip1) CDK ‘inhibitors’ are essential activators of cyclin D-dependent kinases in murine fibroblasts. EMBO J. 1999;18:1571-83.

300. Moller MB. P27 in cell cycle control and cancer. Leuk Lymphoma 2000;39:19-27.

301. Meyer-Siegler K, Hudson P B. Enhanced expression of macrophage migration inhibitory factor in prostatic adenocarcinoma metastases. Urology 1996;48:448-52.

302. Akbar S M, Abe M, Murakami H, Tanimoto K, Kumagi T, Yamashita Y et al. Macrophage migration inhibitory factor in hepatocellular carcinoma and liver cirrhosis; relevance to pathogenesis. Cancer Lett. 2001;171:125-32.

303. Kamimura A, Kamachi M, Nishihira J, Ogura S, Isobe H, Dosaka-Akita H et al. Intracellular distribution of macrophage migration inhibitory factor predicts the prognosis of patients with adenocarcinoma of the lung. Cancer 2000;89:334-41.

304. Markert J M, Fuller C M, Gillespie G Y, Bubien J K, McLean L A, Hong R L et al. Differential gene expression profiling in human brain tumors. Physiol Genomics 2001;5:21-33.

305. Krieger D T, Allen W, Rizzo F, Krieger H P. Characterization of the normal temporal pattern of plasma corticosteroid levels. J Clin Endocrinol Metab 1971;32:266-84.

306. Munck A., Guyre P. M. Glucocorticoid physiology and homeostasis in relation to antiinflammatory actions. In: Schleimer R P, Claman H N, Oronsky A. Anti-inflammatory steroid action. Basic and clinical aspects. San Diego: Academic Press, Inc., 1989:30-47.

307. Naray-Fejes-Toth A, Rosenkranz B, Frolich J C, Fejes-Toth G. Glucocorticoid effect on arachidonic acid metabolism in vivo. J Steroid Biochem. 1988;30:155-9.

308. da Silva J A. Sex hormones and glucocorticoids: interactions with the immune system. Ann.N.Y.Acad Sci 1999;876:102-17.

309. Dunn A J. Cytokine activation of the HPA axis. Ann NY.Acad Sci 2000;917:608-17.

310. Cohen J J. Glucocorticoid-induced apoptosis in the thymus. Semin.Immunol 1992;6:363-9.

311. Suster S, Rosai J. Thymus. In: Sternberg SS. Histology for Pathologists, 2 ed. Philadelphia: Lippincott-Raven Publishers, 1997:687-706.

312. Korsmeyer S J. Bc1-2: a repressor of lymphocyte death. Immunol Today 1992;8:285-8.

313. Rhee K, Reisman D, Bresnahan W, Thompson E A. Glucocorticoid regulation of G1 cyclin-dependent kinase genes in lymphoid cells. Cell Growth Differ. 1995;6:691-8.

314. Reisman D, Thompson E A. Glucocorticoid regulation of cyclin D3 gene transcription and mRNA stability in lymphoid cells. Mol Endocrinol 1995;9:1500-9.

315. Rhee K, Bresnahan W, Hirai A, Hirai M, Thompson E A. c-Myc and cyclin D3 (CcnD3) genes are independent targets for glucocorticoid inhibition of lymphoid cell proliferation. Cancer Res 1995;55:4188-95.

316. Cram E J, Ramos R A, Wang E C, Cha H H, Nishio Y, Firestone G L. Role of the CCAAT/enhancer binding protein-alpha transcription factor in the glucocorticoid stimulation of p21waf1/cip1 gene promoter activity in growth-arrested rat hepatoma cells. J Biol Chem. 1998;273:2008-14.

317. Cha H H, Cram E J, Wang E C, Huang A J, Kasler H G, Firestone G L. Glucocorticoids stimulate p21 gene expression by targeting multiple transcriptional elements within a steroid responsive region of the p21waf1/cip1 promoter in rat hepatoma cells. J Biol Chem. 1998;273:1998-2007.

318. Corroyer S, Nabeyrat E, Clement A. Involvement of the cell cycle inhibitor CIP1/WAF1 in lung alveolar epithelial cell growth arrest induced by glucocorticoids. Endocrinology 1997;138:3677-85.

319. Frost G H, Rhee K, Thompson E A, Jr. Glucocorticoid regulation of thymidine kinase (Tk-1) expression in L929 cells. J Biol Chem. 1993;268:6748-54.

320. Hengst L, Reed S L Translational control of p27Kip1 accumulation during the cell cycle. Science 1996;271:1861-4.

321. Hayes J D, Strange R C. Potential contribution of the glutathione S-transferase supergene family to resistance to oxidative stress. Free Radic.Res 1995;22:193-207.

322. Gutteridge J M, Mitchell J. Redox imbalance in the critically ill. Br Med Bull 1999;55:49-75.

323. Galanos C, Freudenberg M A. Mechanisms of endotoxin shock and endotoxin hypersensitivity. Immunobiology 1993; 187:346-56.

324. Simpson E M, Linder C C, Sargent E E, Davisson M T, Mobraaten L E, Sharp J J. Genetic variation among 129 substrains and its importance for targeted mutagenesis in mice. Nat.Genet 1997; 16:19-27.

325. Mengozzi M, Ghezzi P. Cytokine down-regulation in endotoxin tolerance. Eur Cytokine Netw. 1993;4:89-98.

326. Bundschuh D S, Barsig J, Hartung T, Randow F, Docke W D, Volk H D et al. Granulocyte-macrophage colony-stimulating factor and IFN-gamma restore the systemic TNF-alpha response to endotoxin in lipopolysaccharide-desensitized mice. J Immunol 1997;158:2862-71.

327. Randow F, Syrbe U, Meisel C, Krausch D, Zuckermann H, Platzer C et al. Mechanism of endotoxin desensitization: involvement of interleukin 10 and transforming growth factor beta. J Exp Med 1995; 181:1887-92.

328. Evans G F, Zuckerman S H. Glucocorticoid-dependent and -independent mechanisms involved in lipopolysaccharide tolerance. Eur J Immunol 1991;21:1973-9.

329. Balkhy H H, Heinzel F P. Endotoxin fails to induce IFN-gamma in endotoxin-tolerant mice: deficiencies in both IL-12 heterodimer production and IL-12 responsiveness. J Immunol. 1999;162:3633-8.

330. Weber D J, Wolfson J S, Swartz M N, Hooper D C. Pasteurella multocida infections. Report of 34 cases and review of the literature. Medicine (Baltimore) 1984;63:133-54.

331. Goelz M F, Thigpen J E, Mahler J, Rogers W P, Locklear J, Weigler B J et al. Efficacy of various therapeutic regimens in eliminating Pasteurelia pneumotropica from the mouse. Lab Anim Sci 1996;46:280-5.

332. Schultz M, Tonkonogy S L, Sellon R K, Veitkamp C, Godfrey V L, Kwon J et al. IL-2-deficient mice raised under germfree conditions develop delayed mild focal intestinal inflammation. Am J Physiol 1999;276:G1461-G1472.

333. Contractor N V, Bassiri H, Reya T, Park A Y, Baumgart D C, Wasik M A et al. Lymphoid hyperplasia, autoimmunity, and compromised intestinal intraepithelial lymphocyte development in colitis-free gnotobiotic IL-2-deficient mice. J Immunol 1998;160:385-94.

334. Dinarello C A. Proinflammatory and anti-inflammatory cytokines as mediators in the pathogenesis of septic shock. Chest 1997;112:321S-9S.

335. Vincent J L, Zhang H, Szabo C, Preiser J C. Effects of nitric oxide in septic shock. Am J Respir. Crit Care Med 2000;161:1781-5.

336. Shanley T P, Peters J L, Jones M L, Chensue S W, Kunkel S L, Ward P A. Regulatory effects of endogenous interleukin-1 receptor antagonist protein in immunoglobulin G immune complex-induced lung injury. J Clin Invest 1996;97:963-70.

337. Bresnihan B. The prospect of treating rheumatoid arthritis with recombinant human interleukin-1 receptor antagonist. BioDrugs. 2001; 15:87-97.

338. Matthews J R, Wakasugi N, Virelizier J L, Yodoi J, Hay R T. Thioredoxin regulates the DNA binding activity of NF-kappa B by reduction of a disulphide bond involving cysteine 62. Nucleic Acids Res 1992;20:3821-30.

339. Noiva R. Protein disulfide isomerase: the multifunctional redox chaperone of the endoplasmic reticulum. Semin. Cell Dev Biol 1999; 10:481-93.

340. Holmgren A. Antioxidant function of thioredoxin and glutaredoxin systems. Antioxid.Redox.Signal. 2000;2:811-20.

341. Bardwell J C. Building bridges: disulphide bond formation in the cell. Mol Microbiol. 1994; 14:199-205.

342. The Birth, Assembly and Death of Proteins. In: Alberts B, Bray D, Lewis J, Raff M, Roberts K, Watson J. D. Molecular Biology of the Cell, 3 ed. New York: Garland Publishing Inc., 1994:212-22.

343. Goldenberg D P, Creighton T E. Folding pathway of a circular form of bovine pancreatic trypsin inhibitor. J Mol Biol 1984; 179:527-45.

344. Wedemeyer W J, Welker E, Narayan M, Scheraga H A. Disulfide bonds and protein folding. Biochemistry 2000;39:4207-16.

345. Ewbank J J, Creighton T E. Pathway of disulfide-coupled unfolding and refolding of bovine alpha-lactalbumin. Biochemistry 1993;32:3677-93.

346. Miyajima A, et al. Coordinate regulation of immune and inflammatory responses by T cell-derived lymphokines. FASEB J 1988; 2(9): 2462-73.

347. Bloom B R, et al. Mechanism of a reaction in vitro associated with delayed-type hypersensitivity. Science 1996; 153(731): 80-82.

348. McCabe W R Endotoxin and bacteremia due to gram-negative organisms. N Engl J Med 1970; 283(24): 1342-43.

349. Odink K, et al. Two Calcium-Binding proteins in infiltrate macrophages of rheumatoid arthritis. Nature 1987; 330(6143): 80-82.

350. Oki S, et al. MIF produced by a human T cell hybridoma clone. Lymphokine Cytokine Res. 1991; 10(4): 273-80.

351. Hirose D, et al. MIF produced by a human T cell hybridoma clone. Microbiol Immunol. 1991; 35(3): 235-245.

352. Wistow G J, et al. A macrophage migration inhibitory factor is expressed in the differentiating cells of the eye lens. Proc. Natl. Acad. Sci. USA 1993; 90(4): 1272-5.

353. Weiser W Y, et al. Molecular cloning of a cDNA encoding a human macrophage migration inhibitory factor. Proc. Natl. Acad. Sci. USA 1989; 86(19): 7522-6.

354. Bernhagen J, et al. MIF is a pituitary-derived cytokine that potentiates lethal endotoxemia. Nature 1993; 365 (6448): 756-9; Erratum Nature 1995; 378 (6555): 419.

355. Blocki F A, et al. MIF protein are theta-class glutathione S-transferase homologs. Protein Sci. 1993; 2(12): 2095-102.

356. Blocki F A, et al. Rat liver protein linking chemical and immunological detoxification systems. Nature 1992; 360(6401): 269-70.

What is claimed is:

1. A transgenic mouse having a genome that comprises a disruption of the MIF gene such that the mouse lacks or has reduced levels of functional MIF protein.

2. The transgenic mouse according to claim 1, wherein said mouse has a genetic background that is purebred.

3. The transgenic mouse according to claim 2, wherein said mouse is of the C57B1/6 strain.

4. The transgenic mouse according to claim 1, wherein said disruption of the MIF gene comprises complete deletion of the promoter region and all exon regions.

5. The transgenic mouse according to claim 1, wherein said disruption of the MIF gene comprises complete deletion of the promoter region and all exon regions and said mouse exhibits the same response to endotoxin as a mouse lacking said deletion.

6. A transgenic mouse having a genome that comprises an MIF gene such that the entire MIF gene is flanked by loxP sites in the genome.

7. A transgenic mouse having a genome that comprises a mutation of the MIF gene such that at least one codon in the MIF gene is replaced by a substitute codon, said substitute codon coding for an amino acid different from that encoded by the replaced codon.

8. The transgenic mouse according to claim 7, wherein said substitute codon codes for glycine and replaces the codon for proline at position 1 of the MIF protein.

9. The transgenic mouse according to claim 7, wherein said substitute codon codes for serine and replaces the codon for cysteine at position 60 of the MIF protein.

10. A method of producing a transgenic mouse with a genome comprising a disruption or mutation of the MIF gene such that the mouse lacks or has reduced levels of enzymatically functional MIF protein, wherein said mouse has a genetic background that is limited to a single genetic strain, said method comprising:
a) introducing a targeting vector which disrupts the MIF gene in a mouse embryonic stem cell, thereby producing a transgenic embryonic stem cell with the disrupted MIF gene;
b) selecting the transgenic embryonic stem cell whose genome comprises the disrupted MIF gene;
c) introducing the transgenic embryonic stem cell in b) into a blastocyst, thereby forming a chimeric blastocyst; and
d) introducing the chimeric blastocyst of c) into the uterus of a pregnant or pseudopregnant mouse; wherein said pregnant or pseudopregnant mouse gives birth to a transgenic mouse whose genome comprises a disruption of the MIF gene such that the mouse lacks or has reduced levels of functional MIF protein.

11. The method of claim 10, further comprising:
e) breeding the transgenic mouse with a second mouse to generate progeny having a heterozygous disruption or mutation of the MIF gene, thereby expanding the population of mice having a heterozygous disruption or mutation of the MIF gene; and
f) crossbreeding the progeny to produce a transgenic mouse which lacks a functional MIF gene due to a homozygous disruption or mutation of the MIF gene.

12. A method of using immune system cells of a mouse to generate anti-human MIF antibodies comprising:
a) contacting immune cells of said transgenic mouse with human MIF, wherein said transgenic mouse has a genome comprising a disruption of the mouse MIF gene such that the transgenic mouse lacks or has reduced levels of enzymatically functional MIF protein; and
b) isolating antibodies to the human MIF raised in the transgenic mouse.

13. The method of claim 12 wherein said immune cells are contacted with said MIF by injection of said MIF into said transgenic mouse.

14. A method for screening for an inhibitor of a biological function of MIF that does not act by inhibiting an biochemical activity of MIF comprising:
a) contacting a cell of a transgenic mouse comprising a human MIF gene and no mouse MIF gene with a compound, wherein said transgenic mouse has a genome comprising a change in said human MIF gene such that at least one codon in the MIF gene is replaced by a substitute codon, said substitute codon coding for an amino acid different than that encoded by the replaced codon and forming a mutation reducing or eliminating a biochemical activity of the human MIF protein; and
b) identifying whether the compound affects a biological activity of MIF in the transgenic mouse cells.

15. The method of claim 14 wherein said cells comprise part of an intact transgenic mouse.

16. The method according to claim 14, in which said mutation codes for glycine and replaces proline at position 1 of the MIF protein; and said identified MIF activity is tautomerase activity.

17. A method for investigating an in vitro or in vivo activity of MIF comprising utilizing a cell of a transgenic mouse according to claim 1.

18. The method according to claim 17, wherein the in vivo activity is associated with a neoplastic disorder.

19. The method according to claim 17, wherein the in vivo activity is associated with an inflammatory response.

20. The method according to claim 17, wherein the in vivo activity is investigated in an assay for inhibitors of MIF activity other than inhibitors of tautomerase activity.
